SEQUENCE LISTING (76 sequences)

SEQ ID NO: 1 - DNA, 20 bp, Artificial Sequence
  Description of Artificial Sequence Synthetic primer
gttgatatgttgttttttgg

SEQ ID NO: 2 - DNA, 22 bp, Artificial Sequence
  Description of Artificial Sequence Synthetic primer
aaaccaaagaataaaattatac

SEQ ID NO: 3 - DNA, 22 bp, Artificial Sequence
  Description of Artificial Sequence Synthetic primer
gtataattttatttgttggttt

SEQ ID NO: 4 - DNA, 19 bp, Artificial Sequence
  Description of Artificial Sequence Synthetic primer
acaaacaaaactatctacc

SEQ ID NO: 5 - DNA, 19 bp, Artificial Sequence
  Description of Artificial Sequence Synthetic primer
ggtagatagttttgtttgt

SEQ ID NO: 6 - DNA, 21 bp, Artificial Sequence
  Description of Artificial Sequence Synthetic primer
tccacccacctccttccttcc

SEQ ID NO: 7 - DNA, 21 bp, Artificial Sequence
  Description of Artificial Sequence Synthetic primer
ggaaggaaggaggtgggtgga

SEQ ID NO: 8 - DNA, 18 bp, Artificial Sequence
  Description of Artificial Sequence Synthetic primer
cttctcccccccaacccc

SEQ ID NO: 9 - DNA, 18 bp, Artificial Sequence
  Description of Artificial Sequence Synthetic primer
ggggttgggggggagaag

SEQ ID NO: 10 - DNA, 22 bp, Artificial Sequence
  Description of Artificial Sequence Synthetic primer
aaacgctttcccaaaaccaaac

SEQ ID NO: 11 - DNA, 22 bp, Artificial Sequence
  Description of Artificial Sequence Synthetic primer
gtttggttttgggaaagcgttt

SEQ ID NO: 12 - DNA, 22 bp, Artificial Sequence
  Description of Artificial Sequence Synthetic primer
ccacaaacacgaaaacgatccc

SEQ ID NO: 13 - DNA, 22 bp, Artificial Sequence
  Description of Artificial Sequence Synthetic primer
gggatcgttttcgtgtttgtgg

SEQ ID NO: 14 - DNA, 22 bp, Artificial Sequence
  Description of Artificial Sequence Synthetic primer
ccaccgacccgatccccaaaac

SEQ ID NO: 15 - DNA, 22 bp, Artificial Sequence
  Description of Artificial Sequence Synthetic primer
gttttggggatcgggtcggtgg

SEQ ID NO: 16 - DNA, 19 bp, Artificial Sequence
  Description of Artificial Sequence Synthetic primer
aaaaaaacaaactctcccc

SEQ ID NO: 17 - DNA, 19 bp, Artificial Sequence
  Description of Artificial Sequence Synthetic primer
ggggagagtttgttttttt

SEQ ID NO: 18 - DNA, 21 bp, Artificial Sequence
  Description of Artificial Sequence Synthetic primer
caaaaataacacacaccacac

SEQ ID NO: 19 - DNA, 21 bp, Artificial Sequence
  Description of Artificial Sequence Synthetic primer
gtgtggtgtgtgttatttttg

SEQ ID NO: 20 - DNA, 20 bp, Artificial Sequence
  Description of Artificial Sequence Synthetic primer
cccaaccacccaccccccac

SEQ ID NO: 21 - DNA, 20 bp, Artificial Sequence
  Description of Artificial Sequence Synthetic primer
gtggggggtgggtggttggg

SEQ ID NO: 22 - DNA, 25 bp, Artificial Sequence
  Description of Artificial Sequence Synthetic primer
aataaaataaaacacaacaaacccc

SEQ ID NO: 23 - DNA, 23 bp, Artificial Sequence
  Description of Artificial Sequence Synthetic primer
gattaagttatgtatgtttaagt

SEQ ID NO: 24 - DNA, 24 bp, Artificial Sequence
  Description of Artificial Sequence Synthetic primer
ctaccttccttaaatataataacc

SEQ ID NO: 25 - DNA, 24 bp, Artificial Sequence
  Description of Artificial Sequence Synthetic primer
ggttattatatttaaggaaggtag

SEQ ID NO: 26 - DNA, 24 bp, Artificial Sequence
  Description of Artificial Sequence Synthetic primer
tcctattccattattcctaactac

SEQ ID NO: 27 - DNA, 24 bp, Artificial Sequence
  Description of Artificial Sequence Synthetic primer
gtagttaggaataatggaatagga

SEQ ID NO: 28 - DNA, 24 bp, Artificial Sequence
  Description of Artificial Sequence Synthetic primer
tcctattccattattcctaactac

SEQ ID NO: 29 - DNA, 24 bp, Artificial Sequence
  Description of Artificial Sequence Synthetic primer
ggggggagtatggttgtaaagttg

SEQ ID NO: 30 - DNA, 25 bp, Artificial Sequence
  Description of Artificial Sequence Synthetic primer
ccacttatccctctaaaaaattaaa

SEQ ID NO: 31 - DNA, 25 bp, Artificial Sequence
  Description of Artificial Sequence Synthetic primer
tttaattttttagagggataagtgg

SEQ ID NO: 32 - DNA, 25 bp, Artificial Sequence
  Description of Artificial Sequence Synthetic primer
ttcataaaaaataattacaatcccc

SEQ ID NO: 33 - DNA, 25 bp, Artificial Sequence
  Description of Artificial Sequence Synthetic primer
ggggattgtaattattttttatgaa

SEQ ID NO: 34 - DNA, 25 bp, Artificial Sequence
  Description of Artificial Sequence Synthetic primer
ttacttcctctaaataatcaaattc

SEQ ID NO: 35 - DNA, 25 bp, Artificial Sequence
  Description of Artificial Sequence Synthetic primer
gaatttgattatttagaggaagtaa

SEQ ID NO: 36 - DNA, 25 bp, Artificial Sequence
  Description of Artificial Sequence Synthetic primer
cccttctttctctctctctctctct

SEQ ID NO: 37 - DNA, 25 bp, Artificial Sequence
  Description of Artificial Sequence Synthetic primer
agagagagagagagagaaagaaggg

SEQ ID NO: 38 - DNA, 25 bp, Artificial Sequence
  Description of Artificial Sequence Synthetic primer
ccccccaaaaaatctttaaacctcc

SEQ ID NO: 39 - DNA, 25 bp, Artificial Sequence
  Description of Artificial Sequence Synthetic primer
ggaggtttaaagattttttgggggg

SEQ ID NO: 40 - DNA, 25 bp, Artificial Sequence
  Description of Artificial Sequence Synthetic primer
tatcctacaattcacattaattctc

SEQ ID NO: 41 - DNA, 25 bp, Artificial Sequence
  Description of Artificial Sequence Synthetic primer
gagaattaatgtgaattgtaggata

SEQ ID NO: 42 - DNA, 19 bp, Artificial Sequence
  Description of Artificial Sequence Synthetic primer
actctctctttccctctcc

SEQ ID NO: 43 - DNA, 19 bp, Artificial Sequence
  Description of Artificial Sequence Synthetic primer
ggagagggaaagagagagt

SEQ ID NO: 44 - DNA, 23 bp, Artificial Sequence
  Description of Artificial Sequence Synthetic primer
aacaaaacctcaaaaaaaaaaca

SEQ ID NO: 45 - DNA, 23 bp, Artificial Sequence
  Description of Artificial Sequence Synthetic primer
tgttttttttttgaggttttgtt

SEQ ID NO: 46 - DNA, 19 bp, Artificial Sequence
  Description of Artificial Sequence Synthetic primer
tctcaaaaaaaaacaaacc

SEQ ID NO: 47 - DNA, 26 bp, Artificial Sequence
  Description of Artificial Sequence Synthetic primer
gaaattaattaggatttttttagtaa

SEQ ID NO: 48 - DNA, 25 bp, Artificial Sequence
  Description of Artificial Sequence Synthetic primer
ctttaaactacattcccaaacaacc

SEQ ID NO: 49 - DNA, 25 bp, Artificial Sequence
  Description of Artificial Sequence Synthetic primer
ggttgtttgggaatgtagtttaaag

SEQ ID NO: 50 - DNA, 25 bp, Artificial Sequence
  Description of Artificial Sequence Synthetic primer
ccttccccaccaaaccttcccaacc

SEQ ID NO: 51 - DNA, 25 bp, Artificial Sequence
  Description of Artificial Sequence Synthetic primer
ggttgggaaggtttggtggggaagg

SEQ ID NO: 52 - DNA, 23 bp, Artificial Sequence
  Description of Artificial Sequence Synthetic primer
ggggggttgggttatttttttta

SEQ ID NO: 53 - DNA, 23 bp, Artificial Sequence
  Description of Artificial Sequence Synthetic primer
ggggggttgggttatttttttta

SEQ ID NO: 54 - DNA, 25 bp, Artificial Sequence
  Description of Artificial Sequence Synthetic primer
ttaaactccttaatccatatttcaa

SEQ ID NO: 55 - DNA, 25 bp, Artificial Sequence
  Description of Artificial Sequence Synthetic primer
ttgaaatatggattaaggagtttaa

SEQ ID NO: 56 - DNA, 24 bp, Artificial Sequence
  Description of Artificial Sequence Synthetic primer
aaaaaaccaactactaaataattc

SEQ ID NO: 57 - DNA, 24 bp, Artificial Sequence
  Description of Artificial Sequence Synthetic primer
gaattatttagtagttggtttttt

SEQ ID NO: 58 - DNA, 25 bp, Artificial Sequence
  Description of Artificial Sequence Synthetic primer
ccatccattttcaaaactaattaat

SEQ ID NO: 59 - DNA, 25 bp, Artificial Sequence
  Description of Artificial Sequence Synthetic primer
attaattagttttgaaaatggatgg

SEQ ID NO: 60 - DNA, 25 bp, Artificial Sequence
  Description of Artificial Sequence Synthetic primer
tttaaatatttactactaccaccaa

SEQ ID NO: 61 - DNA, 25 bp, Artificial Sequence
  Description of Artificial Sequence Synthetic primer
ttggtggtagtagtaaatatttaaa

SEQ ID NO: 62 - DNA, 20 bp, Artificial Sequence
  Description of Artificial Sequence Synthetic primer
aaatttacaccctctccccc

SEQ ID NO: 63 - DNA, 20 bp, Artificial Sequence
  Description of Artificial Sequence Synthetic primer
gggggagagggtgtaaattt

SEQ ID NO: 64 - DNA, 19 bp, Artificial Sequence
  Description of Artificial Sequence Synthetic primer
ccccaacccttctcccccc

SEQ ID NO: 65 - DNA, 19 bp, Artificial Sequence
  Description of Artificial Sequence Synthetic primer
ggggggagaagggttgggg

SEQ ID NO: 66 - DNA, 24 bp, Artificial Sequence
  Description of Artificial Sequence Synthetic primer
atactttattttaattaaacaatc

SEQ ID NO: 67 - DNA, 24 bp, Artificial Sequence
  Description of Artificial Sequence Synthetic primer
gattgtttaattaaaataaagtat

SEQ ID NO: 68 - DNA, 25 bp, Artificial Sequence
  Description of Artificial Sequence Synthetic primer
aaaacctcccacttattctacacct

SEQ ID NO: 69 - DNA, 25 bp, Artificial Sequence
  Description of Artificial Sequence Synthetic primer
aggtgtagaataagtgggaggtttt

SEQ ID NO: 70 - DNA, 25 bp, Artificial Sequence
  Description of Artificial Sequence Synthetic primer
aaaaatttctatcctccctaaactc

SEQ ID NO: 71 - DNA, 25 bp, Artificial Sequence
  Description of Artificial Sequence Synthetic primer
gagtttagggaggatagaaattttt

SEQ ID NO: 72 - DNA, 25 bp, Artificial Sequence
  Description of Artificial Sequence Synthetic primer
cctattaataaataaacaatccaac

SEQ ID NO: 73 - DNA, 25 bp, Artificial Sequence
  Description of Artificial Sequence Synthetic primer
gttggattgtttatttattaatagg

SEQ ID NO: 74 - DNA, 23 bp, Artificial Sequence
  Description of Artificial Sequence Synthetic primer
tttcccaaaacaaaaaacactcc

SEQ ID NO: 75 - DNA, 23 bp, Artificial Sequence
  Description of Artificial Sequence Synthetic primer
ggagtgttttttgttttgggaaa

SEQ ID NO: 76 - DNA, 21 bp, Artificial Sequence
  Description of Artificial Sequence Synthetic primer
aaaactaactttcaataaatc

(19) United States
(12) Patent Application Publication
(10) Pub. No.: US 2021/0301341 A1
(43) Pub. Date: Sep. 30, 2021

(54) METHODS FOR MEASURING RIBOSOMAL METHYLATION AGE

(71) Applicant: PRESIDENT AND FELLOWS OF HARVARD COLLEGE, Cambridge, MA (US)

(72) Inventors: Juntao HU, Cambridge, MA (US); Bernardo LEMOS, Cambridge, MA (US); Meng WANG, Beijing (CN)

(73) Assignee: PRESIDENT AND FELLOWS OF HARVARD COLLEGE, Cambridge, MA (US)

(21) Appl. No.: 17/266,492

(22) PCT Filed: Aug. 16, 2019
(86) PCT No.: PCT/US19/46847

§ 371 Date: Feb. 5, 2021

Related U.S. Application Data
(60) Provisional application No. 62/719,257, filed on Aug. 17, 2018.

(51) Int. Cl. C12Q 1/6876; G16B 5/00

(52) U.S. Cl. CPC C12Q 1/6876; C12Q 2600/154; G16B 5/00

(57) ABSTRACT

Described herein are methods for identifying the methylation age of a subject. Additionally, included herein are methods for identifying the age (e.g., the subjects chronological age minus the subjects methylation age) of a subject.

### CROSS-REFERENCE TO RELATED APPLICATIONS

This application is a 371 National Phase Entry of International Patent Application No. PCT/US2019/046847 filed Aug. 16, 2019 which claims benefit under 35 U.S.C. § 119(e) of U.S. Provisional Application No. 62/719,257 filed Aug. 17, 2018, the contents of which are incorporated herein by reference in their entirety.

### FIELD OF THE INVENTION

The field of the invention relates to method for identifying the methylation age of a subject.

### SEQUENCE LISTING

The instant application contains a Sequence Listing which has been submitted electronically in ASCII format and is hereby incorporated by reference in its entirety. Said ASCII copy, created on Jan. 29, 2021, is named 002806-091940WOPT_SL.txt and is 17,473 bytes in size.

### BACKGROUND

Aging is a universal feature exhibited by organisms as diverse as yeasts and humans. However, evolutionarily conserved mechanistic markers of aging have been scarce. Described herein is an age clock built specifically using ribosomal DNA (rDNA), the ultra-conserved DNA segment that functions consistently across all domains of life.

### SUMMARY

The invention described herein is related, in part, to the discovery that the ribosomal clock with DNA methylation accurately predicts age, responds to genetic and environmental interventions that modulate lifespan, and can be applied across distant species. Further analyses revealed an excess of age-associated methylation specifically occurs in the rDNA and tRNA genes relative to changes at other functionally coherent segments of the genome. Data presented herein highlight the key role of the rDNA in aging and reveal an evolutionary conserved ribosomal aging clock. The ribosomal clock can be readily deployed to natural populations in the wild and across the spectrum of eukaryotes.

Accordingly, one aspect of the invention described herein provides a method for determining a methylation age of a biological sample comprising measuring the methylation level of a set of methylation sites on ribosomal DNA (rDNA) of the biological sample and determining the age of the biological sample using a statistical prediction algorithm based on the methylation level.

Another aspect of the invention described herein provides a method for determining a methylation age of a subject comprising collecting a biological sample from the subject, extracting genomic DNA for the collected biological sample, measuring a methylation level of a set of methylation sites on the ribosomal DNA, and determining the methylation age of the subject using a statistical prediction algorithm based on the methylation level.

Another aspect of the invention described herein provides a method for determining a Δage of a subject comprising collecting a biological sample from a subject, extracting genomic DNA for the collected biological sample, measuring a methylation level of a set of methylation sites on the ribosomal DNA, determining the methylation age of the subject using a statistical prediction algorithm based on the methylation level, and comparing the methylation age of the subject to a chronological age of the subject, wherein the Δage is the methylation age of the subject minus the chronological age of the subject.

In one embodiment of any other aspect herein, the biological sample is a blood or tissue sample. Exemplary blood samples include, but are not limited to, whole blood, peripheral blood, or cord blood. Exemplary tissue samples include, but are not limited to, skin tissue, breast tissue, ovarian tissue, liver tissue, kidney tissue, lung tissue, pancreatic tissue, thyroid tissue, thymus tissue, spleen tissue, bone marrow, lymphoid tissue, epithelial tissue, endothelial tissue, ectoderm tissue, nervous tissue, connective tissue, and mesoderm tissue.

In one embodiment of any other aspect herein, the subject is male or female. In one embodiment of any other aspect herein, the subject does not exhibit a risk factor of accelerated aging. In one embodiment of any other aspect herein, the subject exhibits at least one risk factor of accelerated aging. Exemplary risk factors of accelerated aging include use of tobacco products, use of alcohol, exposure to environmental toxins, sedentary lifestyle, obesity, cancer, down syndrome, lack of nutritional intake, poor dietary habit, having complex diseases such as diabetes, CHD, hypertension, hyperlipidemia, and genetic risk predisposition.

In one embodiment of any other aspect herein, the set of methylation sites are the methylation sites in Table 1, Table 2, Table 5, Table 6, Table 7, or Table 8. In one embodiment of any other aspect herein, the set of methylation sites comprise at least 90%, at least 80%, at least 70%, at least 60%, at least 50% of the sites of Table 1, Table 2, Table 5, Table 6, Table 7, or Table 8. In one embodiment of any other aspect herein, the set of methylation sites comprise each of the sites of Table 1, Table 2, Table 5, Table 6, Table 7, or Table 8.

In one embodiment of any other aspect herein, the statistical prediction algorithm comprises: (a) identifying at least two coefficients found in Table 1, Table 2, Table 5, Table 6, Table 7, or Table 8 in a biological sample; (b) multiplying each of the at least two coefficients with its corresponding CpG's methylation level to output a value for each of the at least two coefficients; (c) find a sum of values of (b) for each identified coefficient; (d) adding a recalibration intercept to the summed values of (c); and (e) calculating the natural exponentiation of (d), wherein the exponentiation is the predicted methylation age of the subject.

In one embodiment of any other aspect herein, a Δage greater than zero is an indicator of accelerated aging of the individual.

In one embodiment of any other aspect herein, the method further comprises administering a pro-health therapy to a subject with a Δage greater than zero. In one embodiment of any other aspect herein, the pro-health therapy is a therapy that decreases the methylation age of the subject.

Another aspect of the invention described herein provides a method for determining a methylation age of a cell, the method comprising: extracting genomic DNA from the cell or population thereof; measuring a methylation level of a set of methylation sites found in Table 1, Table 2, Table 5, Table 6, Table 7, or Table 8 on the ribosomal DNA; and determining the methylation age of the cell based on the methylation level.

In one embodiment of any other aspect herein, the cell is a mammalian cell. In one embodiment of any other aspect herein, the cell is a pluripotent cell. In one embodiment of any other aspect herein, the cell is a stem cell. In one embodiment of any other aspect herein, the cell is an induced pluripotent stem cell.

Another aspect of the invention described herein provides a kit comprising probes for detecting methylation sites found in Table 1, Table 2, Table 5, Table 6, Table 7, or Table 8. In one embodiment of any aspect, the set of probes comprise at least 90%, at least 80%, at least 70%, at least 60%, at least 50% of the sites of Table 1, Table 2, Table 5, Table 6, Table 7, or Table 8.

Yet another aspect of the invention described herein provides a system for determining a methylation age related property of a subject, the system comprising: an array; an array reader configured to output methylation levels; a display; a memory containing machine readable medium comprising machine executable code having stored thereon instructions for performing a method; a control system coupled to the memory comprising one or more processors, the control system configured to execute the machine executable code to cause the control system to: receive, from the array reader, a methylation data set related to a methylation level of a blood sample of a subject; determine, based on the methylation data set, a methylation age related property using a regression model trained using subjects with an ethnicity that is the same as the subject's ethnicity; and output, to the display, the methylation age related property.

In one embodiment of any aspect herein, the methylation level of a blood sample of the subject is the method level of leukocytes of the subject.

Another aspect described herein provides method of reducing a methylation age in a subject, the method comprising receiving the results of an assay that diagnoses a subject of having advanced methylation aging and administering at least one pro-health therapy, wherein the pro-health therapy reduces the methylation age of the subject as compared to an appropriate control. In one embodiment, the appropriate control is the methylation age of the subject prior to administration.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, “ribosomal DNA (rDNA)” refers to a nucleotide sequence that encodes ribosomal RNA. Ribosomes are assemblies of proteins and ribosomal RNA that are required to translate mRNA to proteins.

As used herein, the term “methylation marker” or “methylation site” refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

As used herein, the term “gene” refers to a region of genomic DNA associated with a given gene. For example, the region can be defined by a particular gene (such as protein coding sequence exons, intervening introns and associated expression control sequences) and its flanking sequence. It is, however, recognized in the art that methylation in a particular region is generally indicative of the methylation status at proximal genomic sites. Accordingly, determining a methylation status of a gene region can comprise determining a methylation status of a methylation marker within or flanking about 10 bp to 50 bp, about 50 to 100 bp, about 100 bp to 200 bp, about 200 bp to 300 bp, about 300 to 400 bp, about 400 bp to 500 bp, about 500 bp to 600 bp, about 600 to 700 bp, about 700 bp to 800 bp, about 800 to 900 bp, 900 bp to 1 kb, about 1 kb to 2 kb, about 2 kb to 5 kb, or more of a named gene, or CpG position.

As used herein, the term “methylation age” refers to the molecular age of a subject estimated, e.g., based on DNA methylation levels. The “methylation age” described herein is based on the prevalence of specific methylation markers, e.g., listed in Table 1, Table 2, Table 5, Table 6, Table 7, or Table 8. As used herein, “Δage” refers to the subject's chronological age minus the subject's methylation age. As used herein, “chronological age” refers to the number of years since the subject's birth.

As used herein, the term “epigenetic” refers to relating to, being, or involving a modification in gene expression that is independent of DNA sequence. Epigenetic factors include modifications in gene expression that are controlled by changes in DNA methylation and chromatin structure. For example, methylation patterns are known to correlate with gene expression.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., accelerated aging. A subject can be male or female.

A subject can be one who has been previously diagnosed with or identified as having accelerated aging or one or more complications related to accelerated aging, and optionally, have already undergone treatment for accelerated aging (e.g., a pro-health therapy). Alternatively, a subject can also be one who has not been previously diagnosed as having accelerated aging or related complications. For example, a subject can be one who exhibits one or more risk factors for accelerated aging or one or more complications related to accelerated aging or a subject who does not exhibit risk factors.

As used herein, the term “pro-health therapy” refers to the therapeutic for the intended use of decreasing a subject's methylation age. A “pro-health therapy” can decrease a subject's methylation age by at least 1%, by at least 2%, by at least 3%, by at least 4%, by at least 5%, by at least 6%, by at least 7%, by at least 8%, by at least 9%, by at least 10%, by at least 20%, by at least 30%, by at least 40%, by at least 50%, or more as compared to an appropriate control. As used herein, the term “appropriate control” refers to the methylation age of a subject prior to the administration of a pro-health therapeutic.

The term “nucleic acids” as used herein may include any polymer or oligomer of pyrimidine and purine bases, preferably cytosine, thymine, and uracil, and adenine and guanine, respectively. The present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1D present data that show the ribosomal DNA (rDNA) methylation clock is sufficient to predict age in mice. (FIGS. 1A and 1B) Two rDNA methylation clock models. (FIG. 1A) Model 1 (subset 1 as training and subset 2 as test). (FIG. 1B) Model 2 (subset 2 as training and subset 1 as test). (FIG. 1C) Age-associated hypermethylation for rDNA CpGs in mice. Spearman's correlation coefficients with age (ρ_{age}) were displayed for CpGs along the rDNA sequence. The red dots indicate CpGs with significant positive correlation with age (ρ_{age}>0, FDR<0.01), while pink and light blue dots denote non-significant CpGs (FDR>0.01) with positive and negative coefficients, respectively. The green circle highlights CpG 7044. (FIG. 1D) Scatterplot shows the correlation between CpG 7044 methylation and age (ρ=0.78, P<2.2e-16).

FIGS. 2A-2C present data that show age-associated hypermethylation is enriched in rDNA and tRNAs and not universally observed in other genomic segments or functional classes. (FIG. 2A) rDNA CpGs (light red) have significantly higher correlation coefficients with age than the genome-wide background of CpGs (light blue) (Wilcoxon rank sum test, P<2.2e-16). (FIG. 2B) Cumulative distribution of correlation coefficients for several groups of genomic elements: intron, exon, H3K27me3 modification, H3K4me3 modification, gene promoter, CpG island (CGI) and bivalent chromatin. (FIG. 2C) Cumulative distribution of correlation coefficients for promoter segments of genes that are functionally related to ribosomal biogenesis or translation: snoRNA genes, cytoplasmic ribosomal protein genes (cRPGs), mitochondrial ribosomal protein genes (mtRPGs), nucleolus genes and tRNA genes. tRNA CpGs have significantly higher correlation coefficients with age than the genome-wide background of CpGs (P=1.92e-12), although still lower than rDNA CpGs.

FIGS. 3A-3E present data that show the rDNAm clock is responsive to genetic and environmental interventions that modulate lifespan. (FIG. 3A) C57BL/6 mice subject to calorie restriction (CR) have lowered rDNAm age than ad libitum (AL) ones (one-tailed t-test of the differences between rDNAm age and chronological age, P=1.08e-8). (FIG. 3B) The CR effect can be consistently observed in B6D2F1 mice (***P=3.56e-6). (FIG. 3C) Slow-aging growth hormone receptor knockout (GHR KO) mice have significantly lower rDNAm age than WT controls (***P=0.00052). (FIG. 3D) Slow-aging snell dwarf (SD) mice did not show significant differences in rDNAm age relative to wild-type (WT) controls (P=0.30). n.s., not significant. (FIG. 3E) Derived iPSC cell lines have significantly lowered rDNAm ages than their progenitor kidney and lung fibroblasts (**P=0.0014 and *P=0.015).

FIGS. 4A-4E present data that show the rDNAm clock is evolutionarily conserved. (FIG. 4A) Left: the phylogenetic tree of 7 vertebrate species. Right: the numbers of CpGs within 18S, 5.8S, 28S in human, and the numbers of conserved CpGs within these components in other species compared to human. The total numbers of (conserved) CpGs in these three components are also shown. (FIG. 4B) Old individuals have significantly larger rDNAm age than those of the young individuals (one-tailed t-test, P=0.04). The average rDNAm age of young individuals was scaled to 1. (FIG. 4C) GM12878 have larger rDNAm age than H1 (˜8.6 folds, sample size insufficient for test). The average rDNAm age of H1 was scaled to 1. (FIG. 4D) For conserved human-mouse homologous CpGs, the ρage in mice is significantly positively correlated with the methylation differences between old and young individuals (Spearman's ρ=0.42, P=1.73e-11). (FIG. 4E) Similar as (FIG. 4D) but with old and young individuals replaced by B-lymphocyte cell GM12878 and embryo stem cell H1, respectively (Spearman's ρ=0.47, P<2.2e-16).

FIGS. 5A and 5B present data that show the CpG density along human (FIG. 5A) and mouse rDNA (FIG. 5B) sequences.

FIGS. 6A-6D present data that show evaluating the reliability of the analysis procedure. (FIG. 6A) Over 99% rDNA mapped reads can be specifically remapped onto rDNA or a homologous region on chromosome 17, but not other genomic regions. (FIG. 6B) The beta value of methylation level in a linear model (age being response) is strongly correlated (ρ=0.94, P<2.2e-16) with that in a linear mixed-effects model (confounding factors being random effects). (FIG. 6C) The ρ_{age} calculated using mice at all age stages is strongly correlated strongly with that calculated using mice that are elder than 10 months (Spearman's ρ=0.83, P<2.2e-16). (FIG. 6D) The correlation coefficients with age for CpGs from the sense and anti-sense strands of rDNA sequence are highly consistent (Spearman's ρ=0.75, P<2.2e-16).

FIGS. 7A-7C present data that show locations and weights of clock sites selected by the three models. (FIG. 7D) Venn plot showing the numbers of shared and unique clock sites among the models.

FIGS. 8A and 8B present data that show validation of age-association for rDNA CpG sites using two independent datasets. (FIG. 8A) For the Stubbs dataset which includes mice at 1, 14, 27 and 40 weeks old, ρage (Stubbs) was estimated as the correlation coefficients for CpGs with age. A strong positive correlation was observed (Spearman's ρ=0.35, P<2.2e-16) between ρage (Stubbs) and ρage (Petkovich). (FIG. 8B) For the Hahn dataset which includes mice at 5 (young) and 26 (old) month old, a significant positive correlation was also observed (Spearman's ρ=0.24, P=6.38e-9) between the old vs. young methylation differences with ρage (Petkovich).

FIGS. 9A-9J present data that show the differences in rDNA methylation for mice that are subject to environmental and genetic interventions. (FIGS. 9A-9D) Calorie restricted (CR) mice at different age stages are lower than ad libitum (AL) ones with the same or the closest ages. C57BL/6 mice were used. (FIGS. 9E and 9F) Similar as (FIGS. 9A-9D), but with B6D2F1 mice used. (FIG. 9G) The slow-aging full-body growth hormone receptor knockout (GHR KO) mice have significantly lower methylation than their wild-type (WT) control. (FIG. 9H) The slow-aging snell dwarf (SD) mice do not have significantly lower methylation than the WT control. (FIGS. 9I and 9J) The derived iPSC cell lines have significantly lowered rDNA methylation levels than their relative kidney and lung fibroblasts ***P<0.001, n.s. not significantly higher in SD mice.

FIGS. 10A-10J present data that show the correlations between ρage and the changes in rDNA methylation caused by interventions. (FIGS. 10A-10D) C57BL/6 calorie restricted (CR) mice at different age stages were considered (versus ad libitum (AL) ones with the same or the closest ages). (FIGS. 10E and 10F) Same as (FIGS. 10A-10D), but with B6D2F1 mice considered. (FIGS. 10G and 10H) Two slow aging mice models [full-body growth hormone receptor knockout (GHR KO) and snell dwarf (SD)] were considered. (FIGS. 10I and 10J) The derived iPSC cell lines were considered (versus their relative kidney and lung fibroblasts).

FIGS. 11A and 11B present data that show the overall methylation of human rDNA in two datasets. (FIG. 11A) the B-lymphocyte cell GM12878 have significantly higher methylation level than embryo stem cell H1 (***P<0.001). (FIG. 11B) For skin samples, inter-individual variability was observed across individuals, while the sun-exposed and unexposed samples from same individuals have very similar methylation levels. Y18, Y23 and Y25 denote the 3 young individuals (18, 23 and 25 years old), and Y74, Y75 and Y83 denote the 3 old individuals (74, 75 and 83 years old).

FIGS. 12A-12I present data that shows for conserved human-mouse homologous CpGs, the ρage in mice is significantly positively correlated with the methylation differences of every old vs. young comparison in human skins, irrespective of the inter-individual variability. Y18, Y23 and Y25 denote the 3 young individuals (18, 23 and 25 years old), and Y74, Y75 and Y83 denote the 3 old individuals (74, 75 and 83 years old).

FIG. 13 presents data that shows the relationship between error in the estimate and the number of sites included in the model. The samples are randomly split into training and testing sets and then performed the modeling and testing process for 10,000 times. When the included sites are fewer than 50, the models with more sites tend to perform better, while the models with more than 50 sites tend to work similarly to one another.

FIG. 14 presents data that shows prediction of biological age from a human rDNA clock built exclusively on whole blood. The x-axis shows the chronological age and y-axis shows the estimated biological age. The smoothed linear regression line and its 90% confidence intervals are shown in black and grey, respectively.

FIG. 15 presents data that shows prediction of biological age from multi-tissue human rDNA clocks. The x-axis shows the chronological age and y-axis shows the predicted biological age. The smoothed linear regression line and its 90% confidence intervals are shown in black and grey, respectively. (Top) Model calculated with samples across all ages. (Bottom) Model calculated with samples from individuals with chronological age <75 years old. Biological ages as predicted by the model and calculate from samples of the following tissues and cell types: CD14+ cells, CD19+ cells, CD3+ cells, CD4+ cells, CD34+ cells, CD56+ cells, Skin fibroblast, cord blood, liver tissue, normal breast epithelial cells, ovarian granulosa cells, saliva, peripheral blood, whole blood.

FIGS. 16A-16E present data showing the development of the rDNAm age clock. (FIGS. 16A and 16B) Example of two rDNA methylation clock models: (FIG. 16A) Model 1; (FIG. 16B) Model 2. Note that the training and testing subsets are reversed in the two models. (FIGS. 16C and 16D) Performance of 20,000 models trained and tested on randomly split subsets of the mice data set. (FIG. 16C) Correlation coefficients (p) between the predicted age (i.e., rDNAm age) and chronological age of the test subsets were plotted against the number of clock CpGs of each model. (FIG. 16D) The median absolute errors (MAEs) of the rDNAm ages were plotted against the number of clock CpGs of each model. (FIG. 16E) Location and weights of the 72 clock sites identified by the best-fitted model. The three gray blocks represent the 18S, 5.8S, and 28S components (from left to right). The shading represents the strength of age association in each site.

FIG. 17 presents data that shows the occurrence of CpGs in the 20,000 rDNAm models. CpGs are ranked in a descending order based on their occurrence from a total of 786,095 events (considering 1 CpG occurrence in 1 model as an event). Among all the input CpGs, 90.2% (736/816) were selected at least once, with the 200 most frequent CpGs accounting for 95.3% of all the models. The 22 most frequent CpGs accounted for 43.2% of all events and were picked in over half of all models.

FIGS. 18A and 18B presents data that shows performance of the best fitted rDNAm models across mouse and canids. (FIG. 18A) The model trained in canid and tested in mouse. (FIG. 18B) The model trained in mice and tested in canid. Samples from individuals with identical age (mouse) or similar age (canid) were grouped. rDNAm ages were resealed.

### DETAILED DESCRIPTION

The invention described herein is related, in part, to the discovery that the ribosomal clock with DNA methylation more accurately predicts age, responds to genetic and environmental interventions that modulate lifespan and can be applied across distant species, as compared to other methylation clocks, e.g., CpG methylation clocks. Presented herein is are sets of methylation sites present on rDNA that accurately predict the age of a biological sample. By obtaining this biological sample from a subject, one can use the methylation model described herein to predict the methylation age of the subject. Further, the methylation model presented herein can be used to predict the methylation age of a cell, for example, a pluripotent cell. As the age of a pluripotent cell can affect its ability to differentiate, this model is useful is predicting a cell that fit enough to differentiate.

### Methylation Clock

The present invention relates to methods for estimating the methylation age as compared to the chronological and/or biological age of a subject based on measuring DNA Cytosine-phosphate-Guanine (CpG) methylation markers that are attached to our DNA found in whole blood.

One aspect provides a method for determining a methylation age of a biological sample comprising measuring the methylation level of a set of methylation sites on ribosomal DNA (rDNA) of the biological sample; and determining the age of the biological sample using a statistical prediction algorithm based on the methylation level.

One aspect provides a method for determining a methylation age of a subject, the method comprising collecting a biological sample from the subject; extracting genomic DNA for the collected biological sample; measuring a methylation level of a set of methylation sites on the ribosomal DNA; and determining the methylation age of the subject using a statistical prediction algorithm based on the methylation level.

One aspect provides a method for determining a Δage of a subject comprising collecting a biological sample from a subject; extracting genomic DNA for the collected biological sample; measuring a methylation level of a set of methylation sites on the ribosomal DNA; determining the methylation age of the subject using a statistical prediction algorithm based on the methylation level; and comparing the methylation age of the subject to a chronological age of the subject; wherein the Δage is the methylation age of the subject minus the chronological age of the subject.

In one embodiment, a Δage greater than zero is an indicator of accelerated aging in the individual. In one embodiment, a subject that is identified as having accelerated aging is administered a pro-health therapy. In another embodiment, a subject that is identified as having accelerated aging is administered at least one pro-health therapy.

Yet another aspect provides a method for determining a methylation age of a cell comprising extracting genomic DNA from the cell or population thereof; measuring a methylation level of a set of methylation sites found in Table 1 or Table 2 on the ribosomal DNA; and determining the methylation age of the cell based on the methylation level. In one embodiment, the cell is a mammalian cell, a pluripotent cell, a stem cell, or an induced pluripotent stem cell. Methods for obtaining and maintaining such cells are known in the art.

A method of reducing a methylation age in a subject comprising receiving the results of an assay that diagnoses a subject of having advanced methylation aging and administering at least one pro-health therapy, wherein the pro-health therapy reduces the methylation age of the subject as compared to an appropriate control. In one embodiment, the methylation age is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 85%, at least 90%, at least 95%, or more as compared to an appropriate control. As used herein, an “appropriate control” refers to the methylation age of a subject prior to administration of a pro-health therapy. Alternately, an appropriate control can refer to the methylation age of a healthy individual of the same age. Assays that measure the methylation age of a subject are described herein, e.g., using the methylation model as described herein.

As rDNA function is essential to the cell and rDNA dysfunction has been traced to several tissue-specific diseases the rDNA methylation age can also be used to predict a subject's risk of developing a tissue-specific disease of aging (e.g., Alzheimer's, as in cognitive age) or tissue-specific condition of aging (e.g., infertility, as in a fertility age) beyond the measurement of biological age. Thus, methods described herein, e.g., that predict the methylation age of a subject, can further be used to predict the subject's risk of developing an aging-associated disease. As used herein, an “aging-associated disease” refers to a disease that is most often seen with increasing frequency with biological aging. Essentially, “aging-associated diseases” are complications arising from advanced biological aging of a subject and can mean diseases of the elderly. “Aging-associated diseases” do not refer to age-specific diseases, such as the childhood diseases, e.g., chicken pox and measles. Nor should aging-associated diseases be confused with accelerated aging diseases, all of which are genetic disorders. Exemplary aging-associated diseases include but are not limited to atherosclerosis and cardiovascular disease, cancer, arthritis, cataracts, osteoarthritis, osteoporosis, type 2 diabetes, hypertension and Alzheimer's disease. Infertility, a disease characterized by the failure to establish a clinical pregnancy after 12 months of regular, unprotected sexual intercourse or due to an impairment of a person's capacity to reproduce either as an individual or with his/her partner, is associated with aging of a subject. Further, decline in sensory systems (e.g., hearing, visual acuity, vestibular function), muscle strength, immunosenescence (e.g., immune system function), mobility and urologic function are associated with advanced biological aging.

As methylation age is an accurate predictor of the overall aging of a subject, e.g., can predict if the subject is aging more rapidly than their biological age indicates, the methylation age can be used to determine a subject's risk for developing an aging-associated disease. For example, after the biological age of 65, a subject's risk of developing Alzheimer's disease doubles every 5 years, and by the biological age of 85, the risk is −33%. If a subject has a biological age of 60, their risk of developing Alzheimer's disease would be considered low. However, if that subject's methylation age is 66, their true risk of developing Alzheimer's disease would be higher. As another example, a female subject's risk of infertility increases with age; infertility is more abundant after the biological age of 35. A subject having a biological age of 30 would be perceived as having a low risk for infertility. However, if that subject's methylation age is 36, their true risk of infertility would be higher. Using methods for measuring the methylation age of a subject described herein could identify the true risks of the subject for developing an aging-associated disease, and allow for earlier intervention, and/or proper treatment for such disease.

Methylation Sites

In various aspects of the invention, the level of a methylation of a specific site or marker is measured. A methylation marker can be found e.g., in the ribosomal DNA and is measured in a biological sample, for example, a blood sample, obtained from a subject. The methylation level of a subject is used to determine the methylation age of the subject. As used herein, the term “methylation” refers to the covalent attachment of a methyl group at the CS-position of the nucleotide base cytosine within the CpG dinucleotides of gene regulatory region. Hypermethylation refers to the methylation state corresponding to an increased presence of 5-methyl-cytosine (“5-mCyt”) at one or a plurality of CpG dinucleotides within a DNA sequence of a test DNA sample, relative to the amount of 5-mCyt found at corresponding CpG dinucleotides within a normal control DNA sample. The term “methylation state” or “methylation status” or “methylation level” or “the degree of methylation” refers to the presence or absence of 5-mCyt at one or a plurality of CpG dinucleotides within a DNA sequence. As used herein, the terms “methylation status” or “methylation state” or “methylation level” or “degree of methylation” are used interchangeably. A methylation site refers to a sequence of contiguous linked nucleotides that is recognized and methylated by a sequence-specific methylase. Furthermore, a methylation site also refers to a specific cytosine of a CpG dinucleotide in the CpG islands. A methylase is an enzyme that methylates (i.e., covalently attaches a methyl group to) one or more nucleotides at a methylation site.

As used here, the term “CpG islands” are short DNA sequences rich in the CpG dinucleotide and defined as sequences greater than 200 bp in length, with a GC content greater than 0.5 and an observed to expected ratio based on GC content greater than 0.6. See Gardiner-Garden and Frommer, “CpG islands in vertebrate genomes,” J. Mol. Biol. 196(2): 261-282 (1987). CpG islands were associated with the 5′ ends of all housekeeping genes and many tissue-specific genes, and with the 3′ ends of some tissue-specific genes. A few genes contain both the 5′ and the 3′ CpG islands, separated by several thousand base pairs of CpG-depleted DNA. The κ′ CpG islands extended through 5′-flanking DNA, exons, and introns, whereas most of the 3′ CpG islands appeared to be associated with exons. CpG islands are generally found in the same position relative to the transcription unit of equivalent genes in different species, with some notable exceptions. CpG islands have been estimated to constitute 1%-2% of the mammalian genome, and are found in the promoters of all housekeeping genes, as well as in a less conserved position in 40% of genes showing tissue-specific expression. The persistence of CpG dinucleotides in CpG islands is largely attributed to a general lack of methylation of CpG islands, regardless of expression status. The term “CpG site” refers to the CpG dinucleotide within the CpG islands. CpG islands are typically, but not always, between about 0.2 to about 1 kb in length.

In one embodiment, the set of methylation sites used to measure methylation age are selected from the methylation sites listed in Table 1. In one embodiment, the set of methylation sites used to measure methylation age are selected from Version 1 of the methylation sites listed in Table 1. In one embodiment, the set of methylation sites used to measure methylation age are selected from Version 2 of the methylation sites listed in Table 1.

Table 1. Version 1 and version 2 of methylation sites used to measure the methylation age of a subject.

TABLE 1

#Version1: 38CpGs
BK000964.3, 4525, +, CG, CGA
BK000964.3, 4551, +, CG, CGA
BK000964.3, 4417, +, CG, CGG
BK000964.3, 4959, +, CG, CGC
BK000964.3, 4887, +, CG, CGG
BK000964.3, 5790, +, CG, CGC
BK000964.3, 4390, +, CG, CGT
BK000964.3, 5099, +, CG, CGA
BK000964.3, 5572, +, CG, CGG
BK000964.3, 5106, +, CG, CGA
BK000964.3, 5616, +, CG, CGG
BK000964.3, 5055, +, CG, CGA
BK000964.3, 5117, +, CG, CGT
BK000964.3, 4345, +, CG, CGA
BK000964.3, 5114, +, CG, CGG
BK000964.3, 4949, +, CG, CGT
BK000964.3, 4132, +, CG, CGC
BK000964.3, 4935, +, CG, CGG
BK000964.3, 6969, +, CG, CGA
BK000964.3, 6933, +, CG, CGA
BK000964.3, 9312, +, CG, CGC
BK000964.3, 11860, +, CG, CGG
BK000964.3, 10378, +, CG, CGG
BK000964.3, 12685, +, CG, CGC
BK000964.3, 9895, +, CG, CGG
BK000964.3, 10655, +, CG, CGT
BK000964.3, 9315, +, CG, CGT
BK000964.3, 11369, +, CG, CGC
BK000964.3, 8568, +, CG, CGC
BK000964.3, 10547, +, CG, CGA
BK000964.3, 10595, +, CG, CGG
BK000964.3, 10560, +, CG, CGT
BK000964.3, 10796, +, CG, CGA
BK000964.3, 12650, +, CG, CGT
BK000964.3, 9656, +, CG, CGG
BK000964.3, 8461, +, CG, CGG
BK000964.3, 9421, +, CG, CGG
BK000964.3, 9947, +, CG, CGA
#Version2: 46CpGs
BK000964.3, 4551, +, CG, CGA
BK000964.3, 4417, +, CG, CGG
BK000964.3, 4959, +, CG, CGC
BK000964.3, 5111, +, CG, CGG
BK000964.3, 4887, +, CG, CGG
BK000964.3, 5790, +, CG, CGC
BK000964.3, 4390, +, CG, CGT
BK000964.3, 5581, +, CG, CGG
BK000964.3, 5099, +, CG, CGA
BK000964.3, 5572, +, CG, CGG
BK000964.3, 5106, +, CG, CGA
BK000964.3, 5616, +, CG, CGG
BK000964.3, 5286, +, CG, CGG
BK000964.3, 5055, +, CG, CGA
BK000964.3, 5117, +, CG, CGT
BK000964.3, 4345, +, CG, CGA
BK000964.3, 4939, +, CG, CGG
BK000964.3, 5114, +, CG, CGG
BK000964.3, 4949, +, CG, CGT
BK000964.3, 4132, +, CG, CGC
BK000964.3, 4377, +, CG, CGA
BK000964.3, 4935, +, CG, CGG
BK000964.3, 6969, +, CG, CGA
BK000964.3, 9312, +, CG, CGC
BK000964.3, 11860, +, CG, CGG
BK000964.3, 10378, +, CG, CGG
BK000964.3, 12685, +, CG, CGC
BK000964.3, 9895, +, CG, CGG
BK000964.3, 10655, +, CG, CGT
BK000964.3, 10780, +, CG, CGG
BK000964.3, 9315, +, CG, CGT
BK000964.3, 11369, +, CG, CGC
BK000964.3, 8472, +, CG, CGA
BK000964.3, 8568, +, CG, CGC
BK000964.3, 11436, +, CG, CGC
BK000964.3, 10547, +, CG, CGA
BK000964.3, 10595, +, CG, CGG
BK000964.3, 10560, +, CG, CGT
BK000964.3, 9839, +, CG, CGA
BK000964.3, 10796, +, CG, CGA
BK000964.3, 12650, +, CG, CGT
BK000964.3, 9656, +, CG, CGG
BK000964.3, 8461, +, CG, CGG
BK000964.3, 9421, +, CG, CGG
BK000964.3, 12560, +, CG, CGC
BK000964.3, 9947, +, CG, CGA

In one embodiment, the set of methylation markers consists of, consists essentially of, or comprises 100% of the methylation sites selected from the sites in Version 1 or Version 2 listed in Table 1. In one embodiment, the set of methylation markers consists of, consists essentially of, or comprises at least 50% of the methylation sites selected from the sites in Version 1 or Version 2 listed in Table 1. In one embodiment, the set of methylation markers consists of, consists essentially of, or comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites in Version 1 or Version 2 listed in Table 1. One skilled in the art can determine if a biological sample is methylated at a methylation site listed in Table 1, e.g., using whole genome sequencing or methods further described herein below.

In one embodiment, the set of methylation markers consists of, consists essentially of, or comprises at of all 38 methylation sites selected from the sites in Version 1 listed in Table 1. In one embodiment, the set of methylation markers consists of, consists essentially of, or comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 methylation sites selected from sites in Version 1 listed in Table 1.

In one embodiment, the set of methylation markers consists of, consists essentially of, or comprises at of all 46 methylation sites selected from the sites in Version 2 listed in Table 1. In one embodiment, the set of methylation markers consists of, consists essentially of, or comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 methylation sites selected from sites in Version 2 listed in Table 1.

In one embodiment, the set of methylation sites used to measure methylation age are selected from the accessible methylation sites listed in Table 2. As used herein, the term “accessible model” refers to a list of methylation sites that are easily measured via standard approaches, e.g., PCR based screening. One skilled in the art will be able to perform PCR-based screening to measure the sites listed in accessible Model 1 or 2 listed in Table 2. In one embodiment, the accessible sites listed in Table 2 are measured using primers listed in Table 4. An accessible model described herein can be used to measure methylation sites as a lower cost than, for example, performing whole genome sequencing. In one embodiment, the set of methylation sites used to measure methylation age are selected from accessible Model 1 listed in Table 2. In one embodiment, the set of methylation sites used to measure methylation age are selected from accessible Model 1 listed in Table 3.

Table 2: Accessible Model 1 and Accessible Model 2 of methylation sites used to measure the methylation age of a subject.

TABLE 2

| | | |
|---|---|---|
| mouse_coordinate | human_coordinate | |
| (BK000964.3) | (U13369.1) | Notes |

Accessible Model 1

| | | |
|---|---|---|
| 5572 | 5221 | |
| 5616 | 5265 | |
| 5790 | 5441 | Unique to accessible model 1 |
| 6933 | 6679 | |
| 10270 | 10314 | |
| 10560 | 10606 | |
| 10595 | 10641 | |
| 11927 | 12057 | |
| 12650 | 12782 | |
| 12697 | 12829 | |

Accessible Model 2

| | | |
|---|---|---|
| 5318 | 4967 | |
| 5520 | 5169 | |
| 5572 | 5221 | shared with accessible Model 1 |
| 5616 | 5265 | shared with accessible Model 1 |
| 6933 | 6679 | shared with accessible Model 1 |
| 6969 | 6715 | |
| 8808 | 8604 | |
| 8828 | 8624 | |
| 10270 | 10314 | shared with accessible Model 1 |
| 10560 | 10606 | shared with accessible Model 1 |
| 10595 | 10641 | shared with accessible Model 1 |
| 10655 | 10701 | |
| 11927 | 12057 | shared with accessible Model 1 |
| 12650 | 12782 | shared with accessible Model 1 |
| 12697 | 12829 | shared with accessible Model 1 |

In one embodiment, the set of methylation markers consists of, consists essentially of, or comprises 100% of the methylation sites selected from the sites listed in Model 1 or Model 2 listed in Table 2. In one embodiment, the set of methylation markers consists of, consists essentially of, or comprises at least 50% of the methylation sites selected from the sites listed in Table 2. In one embodiment, the set of methylation markers consists of, consists essentially of, or comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 2. One skilled in the art can determine if a biological sample is methylated at a methylation site listed in Table 2, e.g., PCR-based assays using primers listed in Table 4, or methods further described herein below.

In one embodiment, the set of methylation markers consists of, consists essentially of, or comprises at of all 10 methylation sites selected from the sites in Accessible Model 1 listed in Table 2. In one embodiment, the set of methylation markers consists of, consists essentially of, or comprises at least 1, 2, 3, 4, 5, 6, 7, 8, or 9 methylation sites selected from sites in Accessible Model 1 listed in Table 2.

In one embodiment, the set of methylation markers consists of, consists essentially of, or comprises at of all 15 methylation sites selected from the sites in Accessible Model 2 listed in Table 2. In one embodiment, the set of methylation markers consists of, consists essentially of, or comprises at least 1, 2, 3, 4, 5, 6, 7, 8, 9, 10, 11, 12, 13, or 14 methylation sites selected from sites in Accessible Model 2 listed in Table 2.

In one embodiment, the set of methylation sites used to measure methylation age are selected from the methylation sites listed in Table 5.

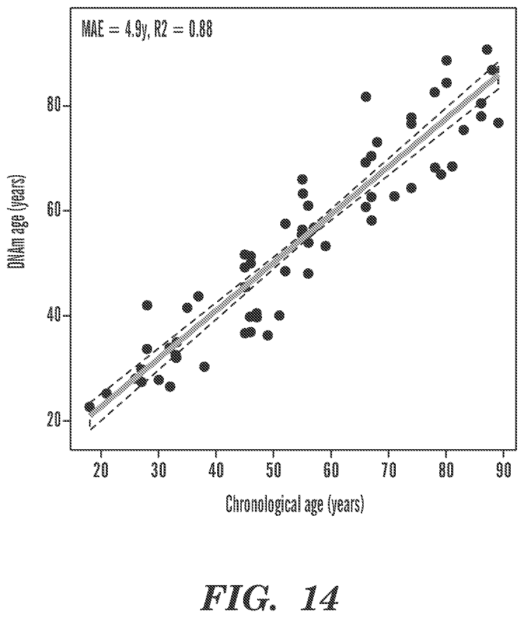

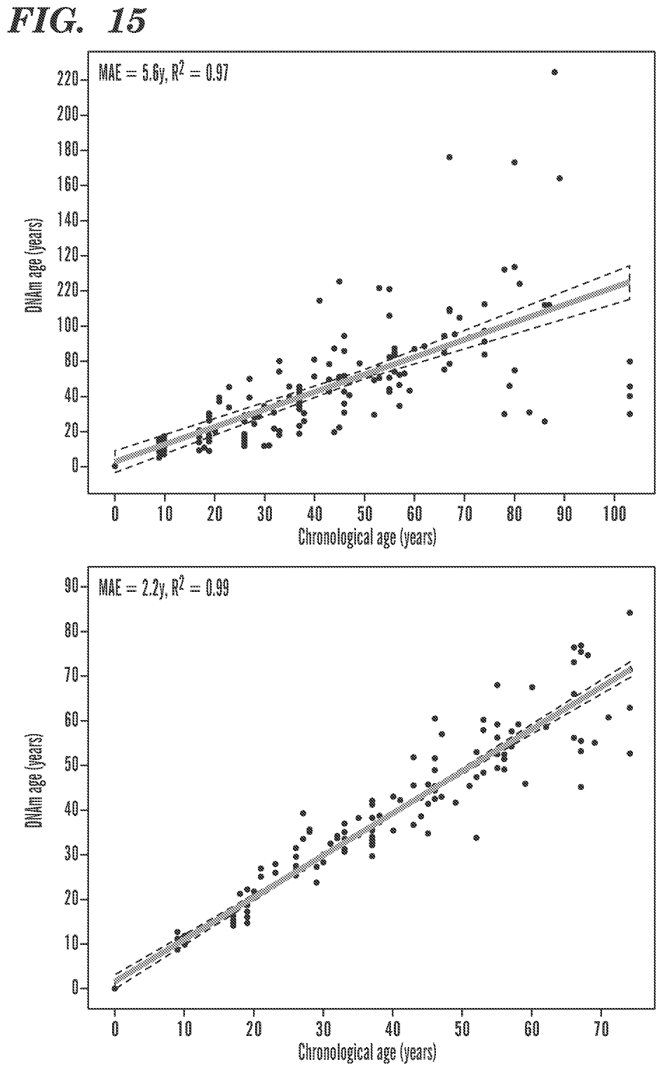

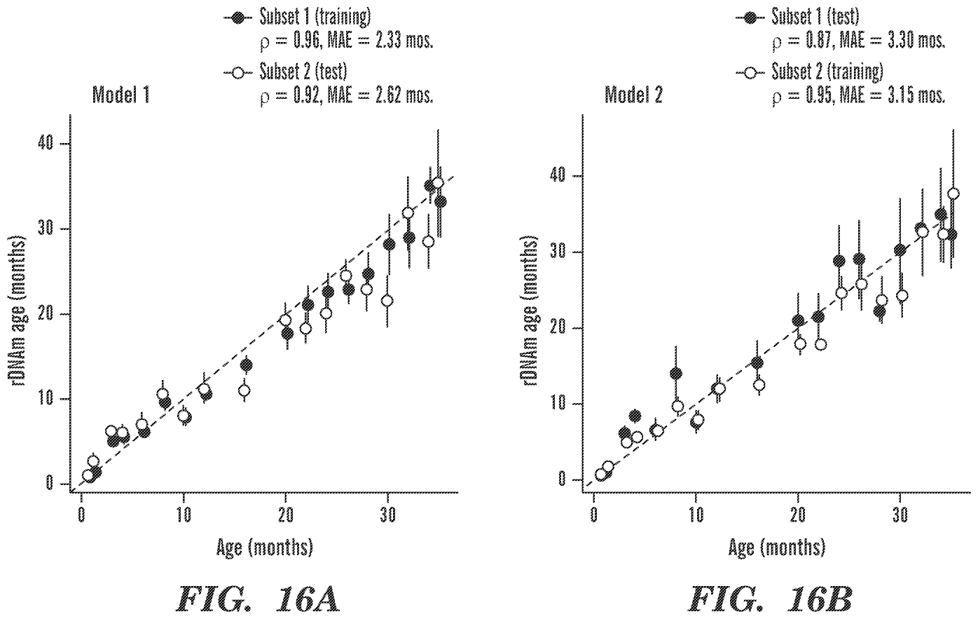

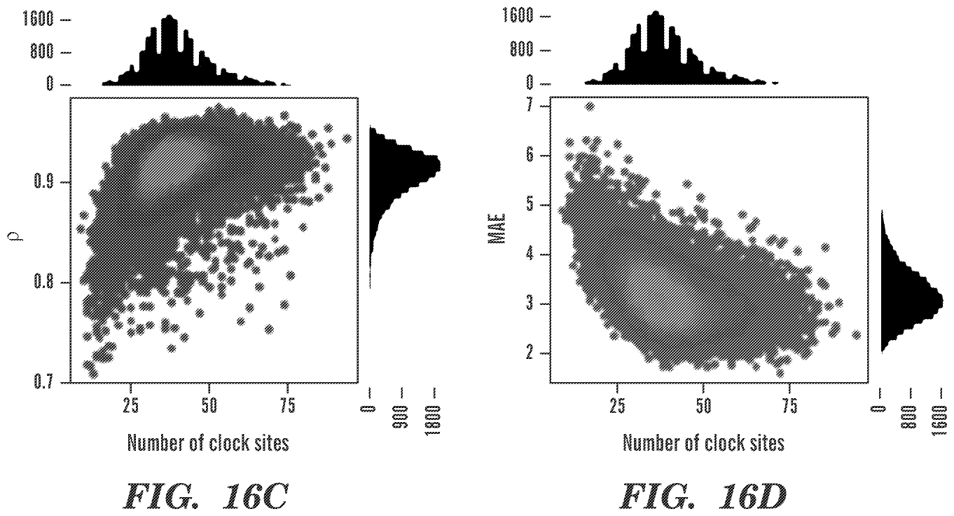

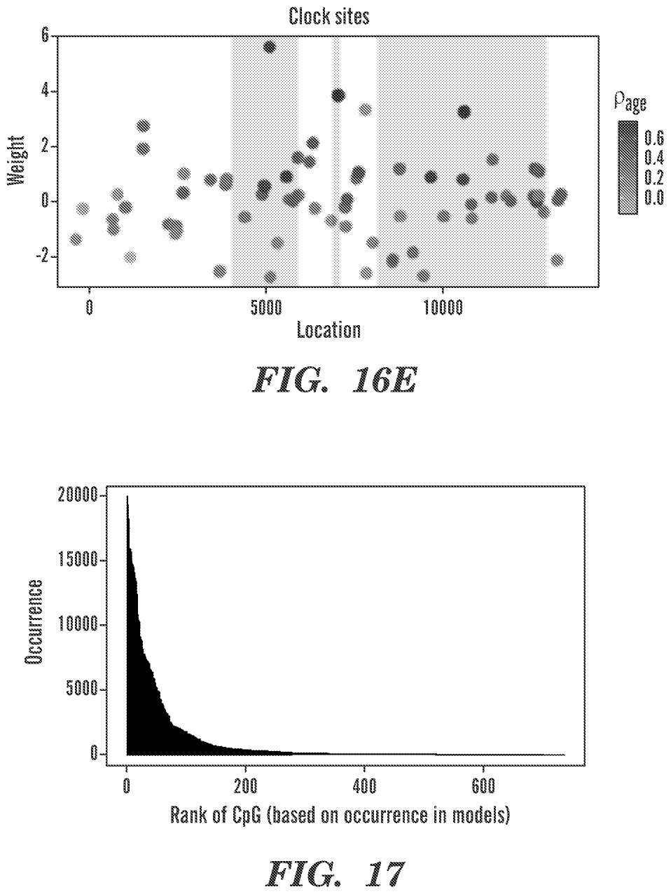

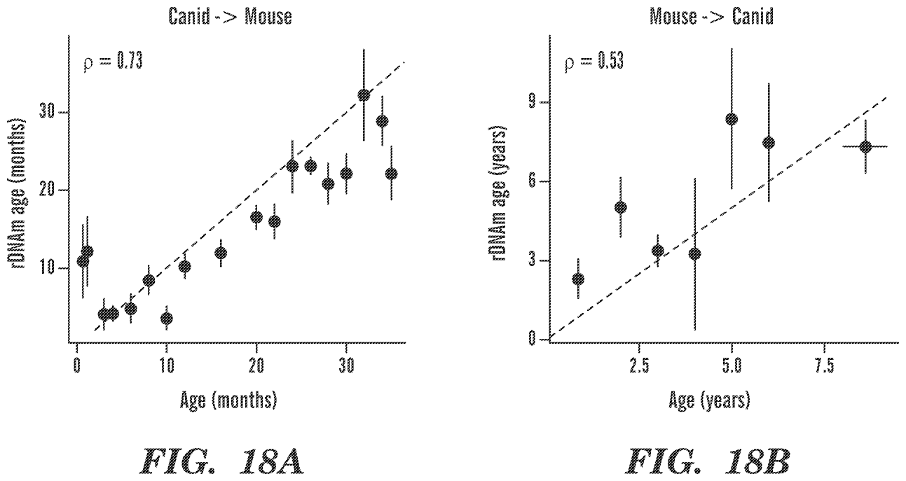

TABLE 5
Clock sites for all tissue in young model, e.g., found in FIG. 14.

(Intercept) 6.14876454543313
U13369.1_899 −1.5283508610121
U13369.1_1580 −1.27297939474452
U13369.1_1592 −2.49515369574745
U13369.1_2015 −2.30026467800161
U13369.1_2098 −1.31978445615417
U13369.1_2939 1.36770071959168
U13369.1_3563 1.8918918368918
U13369.1_3572 0.765403216180827
U13369.1_3583 1.75233603153705
U13369.1_3598 0.707975457133787
U13369.1_3600 0.290232584543083
U13369.1_3625 0.469734280673269
U13369.1_3729 −1.6875341092783
U13369.1_3934 0.0030223743138273
U13369.1_3937 1.41960850353759
U13369.1_4781 0.332795575865793
U13369.1_4782 2.16092063952762
U13369.1_6448 1.84213300519578
U13369.1_8293 −5.64786973922537
U13369.1_9298 1.82549426700235
U13369.1_9301 0.945746920345054
U13369.1_9369 0.00352041445630262
U13369.1_9836 −0.371009819658935
U13369.1_9842 −0.819171943032402
U13369.1_9845 −1.45581095656605
U13369.1_15196 0.562124989079256
U13369.1_15197 0.2701246980068
U13369.1_15209 3.34263384775902
U13369.1_15210 0.976873056199694
U13369.1_15212 2.27916957474917
U13369.1_15220 0.89456515022796
U13369.1_17763 −2.71624526703439
U13369.1_18062 −0.602119899191946
U13369.1_18738 0.0138438256499973
U13369.1_18760 0.0585006232145591
U13369.1_18768 1.24063240950161
U13369.1_18797 0.573821788338398
U13369.1_18805 1.42236865326742
U13369.1_18854 5.92404584066842
U13369.1_18871 9.09783887261809
U13369.1_19725 −5.4667605550903
U13369.1_19726 −1.96311040153072
U13369.1_19811 −0.00917221728530959
U13369.1_19835 4.40785686353667
U13369.1_19872 −3.95528413049568
U13369.1_19881 6.39427013241746
U13369.1_19884 −0.699640085455106
U13369.1_19888 −1.90074437088381
U13369.1_19919 −1.57973753020815
U13369.1_19972 −3.12887190990982
U13369.1_19977 −0.672634522781668
U13369.1_19981 −0.0138943871169307
U13369.120004 −0.727120339695311
U13369.120719 −2.53779885743912
U13369.121333 −1.78902567973873
U13369.121696 −0.292511167552859
U13369.123749 2.22960321412972
U13369.127462 −1.20768746290638
U13369.127476 8.18305866161731
U13369.128437 −0.0671214503670922
U13369.1_31267 −10.6445424135929
U13369.1_31748 −1.62389434971157
U13369.1_32811 −2.1680850642709
U13369.1_32843 −2.8087971724593
U13369.1_32844 −0.235353749584663
U13369.1_32896 −0.282648922963513
U13369.1_34658 3.51062252506539
U13369.1_35718 −1.47449989490245
U13369.1_36062 −0.863309550345492
U13369.1_36123 −5.34661401417284
U13369.1_36284 0.259966464622566
U13369.1_36755 −3.3897418199584
U13369.1_36779 −1.15192427539261
U13369.1_37029 7.85938155908198
U13369.1_38126 −0.245733167114578
U13369.1_38450 3.84670783033133
U13369.1_38497 3.06133892752078
U13369.1_38501 −1.4180553524762
U13369.1_38983 −0.51258797998627
U13369.1_42161 −2.44463798770329

In one embodiment, the set of methylation markers consists of, consists essentially of, or comprises 100% of the methylation sites selected from the sites listed in Table 5. In one embodiment, the set of methylation markers consists of, consists essentially of, or comprises at least 50% of the methylation sites selected from the sites listed in Table 5. In one embodiment, the set of methylation markers consists of, consists essentially of, or comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 5. One skilled in the art can determine if a biological sample is methylated at a methylation site listed in Table 5, e.g., using methods further described herein below.

In one embodiment, the set of methylation markers consists of, consists essentially of, or comprises at of all 80 methylation sites selected from Table 5. In one embodiment, the set of methylation markers consists of, consists essentially of, or comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 methylation sites selected from sites of Table 5.

In one embodiment, the set of methylation sites used to measure methylation age are selected from the methylation sites listed in Table 6.

TABLE 6
Clock sites for all tissue in all ages model, e.g., found in FIG. 14.

(Intercept) 5.29384180153
U13369.1_899 −0.381241341459375
U13369.1_1592 −2.71558485689569
U13369.1_2015 −0.676310456131667
U13369.1_2231 −2.02006749427548
U13369.1_2939 0.182823345731792
U13369.1_3563 2.86978864925013
U13369.1_3583 1.99547977615636
U13369.1_3597 0.389385085257266
U13369.1_3598 0.259517648757238
U13369.1_3600 1.36982025398051
U13369.1_3729 −1.05475944790051
U13369.1_6823 −0.164280910490165
U13369.1_7886 0.444040785988251
U13369.1_8270 −0.889389419426754
U13369.1_8293 −6.13919947849529
U13369.1_9298 1.62513956458489
U13369.1_9842 −0.0553296959998889
U13369.1_9845 −0.642050253062744
U13369.1_10462 0.23516556046783
U13369.1_15196 1.76342555284873
U13369.1_15197 0.997885493869236
U13369.1_15202 0.259225051372982
U13369.1_15209 4.4488948185526
U13369.1_15212 0.647498235924569
U13369.1_15220 0.0411489479623403
U13369.1_17007 2.50195769647134
U13369.1_17763 −1.05463150454339
U13369.1_18062 −0.224355823691386
U13369.1_18135 0.0029663419287276
U13369.1_18836 0.454420496461288
U13369.1_18854 0.0334241898422056
U13369.1_18871 13.5811954250193
U13369.1_19725 −0.117026753166523
U13369.1_19872 −2.56648139714728
U13369.1_19881 6.50031285115463
U13369.1_19956 −2.44956763516021
U13369.1_19972 −2.36270035739937
U13369.1_19981 −1.28474903535898
U13369.1_20719 −1.98289132867832
U13369.1_21333 −1.03055910211935
U13369.1_21696 −0.610566613617413
U13369.1_23728 2.18944806217132
U13369.1_27462 −0.411838466894176
U13369.1_27476 6.61132600124943
U13369.1_27562 0.000341641855678721
U13369.1_28437 −0.730396134969019
U13369.1_31267 −4.70506508047744
U13369.1_31277 −2.62166424930714
U13369.1_31748 −1.43070116359545
U13369.1_32811 −0.508260954276714
U13369.1_32843 −1.89181649507113
U13369.1_32896 −2.21006465955519
U13369.1_32964 −0.442925443580126
U13369.1_35735 −0.219678823177837
U13369.1_35814 −3.14176177382902
U13369.1_36123 −3.74353436233419
U13369.1_36755 −0.431911234061614
U13369.1_36779 −0.499356143661338
U13369.1_37029 2.67075461014074
U13369.1_37408 −1.86645378021259
U13369.1_38363 −0.865623818116829
U13369.1_38450 0.621051649426421
U13369.1_38497 3.2441051661159
U13369.1_38501 −1.35139907596813
U13369.1_38983 −0.556677682592627
U13369.1_39012 −0.204393078971139
U13369.1_42161 −0.668387441544272

In one embodiment, the set of methylation markers consists of, consists essentially of, or comprises 100% of the methylation sites selected from the sites listed in Table 6. In one embodiment, the set of methylation markers consists of, consists essentially of, or comprises at least 50% of the methylation sites selected from the sites listed in Table 6. In one embodiment, the set of methylation markers consists of, consists essentially of, or comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 6. One skilled in the art can determine if a biological sample is methylated at a methylation site listed in Table 6, e.g., using methods further described herein below.

In one embodiment, the set of methylation markers consists of, consists essentially of, or comprises at of all 67 methylation sites selected from Table 6. In one embodiment, the set of methylation markers consists of, consists essentially of, or comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67 methylation sites selected from sites of Table 6.

In one embodiment, the set of methylation sites used to measure methylation age are selected from the methylation sites listed in Table 7.

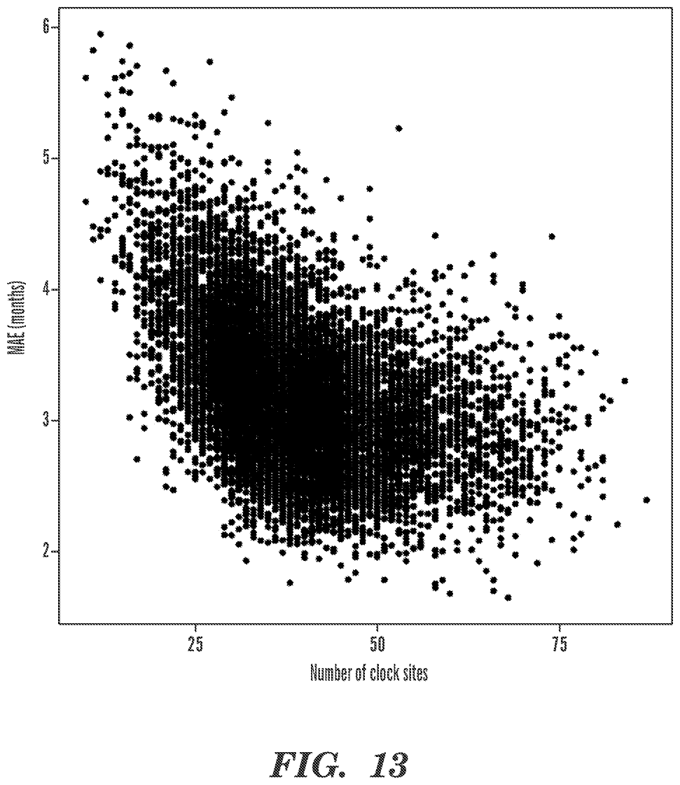

TABLE 7
Clock sites all blood sample, e.g., found in FIG. 13.

(Intercept) 9.41750239621534
U13369.1_845 −0.240004375917871
U13369.1_4383 0.06225590305959
U13369.1_5584 0.0118378504131981
U13369.1_7885 0.0500288977964654
U13369.1_7937 0.136521754452594
U13369.1_8110 0.215117745754251
U13369.1_10430 0.361158838888179
U13369.1_11487 0.278613939664927
U13369.1_12388 −26.4533382558714
U13369.1_12765 0.00372330356304205
U13369.1_38250 −2.87589483281089
U13369.1_38496 0.0123148248919918
U13369.1_38809 0.362796349644508
U13369.1_38922 −0.383088688528103
U13369.1_39149 −1.39112905993143
U13369.1_39371 1.23764994010128
U13369.1_40469 −0.171687138256268
U13369.1_40806 1.02343705289223
U13369.1_40813 0.32481253143942
U13369.1_40847 −0.00717245146192061
U13369.1_40998 −1.73401044392292
U13369.1_41002 −2.34885769576828
U13369.1_42031 0.284506065003307
U13369.1_42032 0.0112202409792738
U13369.1_42162 −0.0599042623327479
U13369.1_42226 −0.00863045875474819
U13369.1_42235 −0.175600572257879

In one embodiment, the set of methylation markers consists of, consists essentially of, or comprises 100% of the methylation sites selected from the sites listed in Table 7. In one embodiment, the set of methylation markers consists of, consists essentially of, or comprises at least 50% of the methylation sites selected from the sites listed in Table 7. In one embodiment, the set of methylation markers consists of, consists essentially of, or comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 7. One skilled in the art can determine if a biological sample is methylated at a methylation site listed in Table 7, e.g., using methods further described herein below.

In one embodiment, the set of methylation markers consists of, consists essentially of, or comprises at of all 27 methylation sites selected from Table 7. In one embodiment, the set of methylation markers consists of, consists essentially of, or comprises at least 1, 2, 3, 4, 5, 6, 7, 8, 9, 10, 11, 12, 13, 14, 15, 16, 17, 18, 19, 20, 21, 22, 23, 24, 25, 26 or 27 methylation sites selected from sites of Table 7.

In one embodiment, the set of methylation sites used to measure methylation age are selected from the methylation sites listed in Table 8.

TABLE 8

| | | Occurrence | Rhoage |
|---|---|---|---|
| Reference | coordinate | in 20,000 models | (age-association) |
| BK000964.3 | 10595 | 19957 | 0.746007488 |
| BK000964.3 | 7044 | 19901 | 0.777952102 |
| BK000964.3 | 5099 | 19272 | 0.616619806 |
| BK000964.3 | 9656 | 18176 | 0.665918582 |
| BK000964.3 | 2286 | 16519 | 0.701588417 |
| BK000964.3 | 6318 | 15925 | 0.65051261 |
| BK000964.3 | 3852 | 15868 | 0.446316774 |
| BK000964.3 | 7830 | 15675 | −0.016671219 |
| BK000964.3 | 5616 | 14862 | 0.098256912 |
| BK000964.3 | −179 | 14825 | −0.094363128 |
| BK000964.3 | 6113 | 14648 | 0.642463111 |
| BK000964.3 | 1527 | 14469 | 0.481867469 |
| BK000964.3 | 7290 | 14194 | 0.64265105 |
| BK000964.3 | −375 | 14059 | 0.201143503 |
| BK000964.3 | 7833 | 13669 | 0.089269396 |
| BK000964.3 | 5572 | 13580 | 0.628142822 |
| BK000964.3 | 1159 | 13387 | −0.171063178 |
| BK000964.3 | 10811 | 12353 | 0.144931253 |
| BK000964.3 | 7614 | 10874 | 0.684007455 |
| BK000964.3 | 13321 | 10499 | 0.563907922 |
| BK000964.3 | 1293 | 10390 | −0.083872859 |
| BK000964.3 | 2653 | 10266 | 0.544686114 |
| BK000964.3 | 9947 | 9854 | 0.104448834 |
| BK000964.3 | 6933 | 9121 | −0.094496452 |
| BK000964.3 | 5318 | 8907 | −0.051460076 |
| BK000964.3 | 12650 | 8796 | 0.541001166 |
| BK000964.3 | 2427 | 8482 | 0.196723578 |
| BK000964.3 | 3359 | 8141 | 0.010640374 |
| BK000964.3 | 13219 | 7787 | 0.225886021 |
| BK000964.3 | 8568 | 7774 | 0.202839989 |
| BK000964.3 | 11927 | 7537 | 0.153668743 |
| BK000964.3 | 3688 | 7495 | 0.28685548 |
| BK000964.3 | 2479 | 7382 | 0.250307197 |
| BK000964.3 | 7807 | 7297 | −0.08828775 |
| BK000964.3 | 8787 | 7237 | −0.07314355 |
| BK000964.3 | 8003 | 7187 | 0.227329125 |
| BK000964.3 | 4885 | 7092 | 0.330033087 |
| BK000964.3 | 9444 | 7077 | 0.110100432 |
| BK000964.3 | 9142 | 6977 | 0.223446356 |
| BK000964.3 | 11778 | 6647 | 0.076169034 |
| BK000964.3 | 685 | 6557 | 0.310034076 |
| BK000964.3 | 10655 | 6525 | 0.293169898 |
| BK000964.3 | 12697 | 6382 | 0.140405612 |
| BK000964.3 | 2269 | 6354 | 0.692723396 |
| BK000964.3 | 10560 | 6002 | 0.5640623 |
| BK000964.3 | 248 | 5945 | 0.072531069 |
| BK000964.3 | 5479 | 5838 | −0.080535262 |
| BK000964.3 | 11115 | 5748 | 0.105239274 |
| BK000964.3 | −390 | 5515 | 0.079070343 |
| BK000964.3 | 10411 | 5216 | 0.070050944 |
| BK000964.3 | 10019 | 5180 | 0.466036956 |
| BK000964.3 | 9436 | 5154 | −0.123826698 |
| BK000964.3 | 3355 | 4908 | 0.195899666 |
| BK000964.3 | 9315 | 4886 | 0.165173301 |
| BK000964.3 | 2696 | 4813 | −0.049018545 |
| BK000964.3 | 831 | 4519 | 0.218146957 |
| BK000964.3 | 2863 | 4267 | 0.148351073 |
| BK000964.3 | 11860 | 4228 | −0.031301929 |
| BK000964.3 | 4887 | 3964 | 0.286303649 |
| BK000964.3 | 9246 | 3953 | 0.040546183 |
| BK000964.3 | 5900 | 3910 | 0.456499047 |
| BK000964.3 | 11022 | 3777 | 0.528597185 |
| BK000964.3 | 7822 | 3608 | 0.070373126 |
| BK000964.3 | 5919 | 3535 | 0.330198935 |
| BK000964.3 | 4417 | 3411 | −0.001468274 |
| BK000964.3 | 7478 | 3400 | 0.664163365 |
| BK000964.3 | 2936 | 3312 | 0.178255205 |
| BK000964.3 | 155 | 3205 | 0.10032592 |
| BK000964.3 | 4959 | 3202 | 0.595157832 |
| BK000964.3 | 13250 | 3188 | 0.588551437 |
| BK000964.3 | 618 | 3005 | −0.009385429 |
| BK000964.3 | 7838 | 2955 | 0.001463241 |
| BK000964.3 | 5117 | 2559 | 0.098434783 |
| BK000964.3 | 1313 | 2499 | −0.048236584 |
| BK000964.3 | 11369 | 2452 | 0.240559938 |
| BK000964.3 | 642 | 2378 | 0.051398914 |
| BK000964.3 | 12693 | 2311 | 0.058916441 |
| BK000964.3 | 10378 | 2291 | 0.179235173 |
| BK000964.3 | 769 | 2205 | 0.067139566 |
| BK000964.3 | 3392 | 2188 | 0.617942092 |
| BK000964.3 | 4390 | 2175 | 0.147198268 |
| BK000964.3 | 9234 | 2149 | 0.196203389 |
| BK000964.3 | 11396 | 2137 | 0.264683921 |
| BK000964.3 | 1522 | 2110 | 0.467476704 |
| BK000964.3 | 11770 | 2094 | 0.196395852 |
| BK000964.3 | 5111 | 2068 | 0.536483915 |
| BK000964.3 | 170 | 2055 | 0.128208029 |
| BK000964.3 | 7952 | 2028 | 0.279650029 |
| BK000964.3 | 11366 | 2025 | 0.35383292 |
| BK000964.3 | 4908 | 2022 | 0.319105762 |
| BK000964.3 | 13330 | 1983 | 0.443440635 |
| BK000964.3 | 5790 | 1940 | 0.363635315 |
| BK000964.3 | 1587 | 1906 | 0.234195614 |
| BK000964.3 | 5106 | 1905 | 0.06140239 |
| BK000964.3 | 9456 | 1889 | −0.008995907 |
| BK000964.3 | 660 | 1809 | 0.314863081 |
| BK000964.3 | 1769 | 1791 | −0.187892118 |
| BK000964.3 | 11436 | 1765 | 0.294120747 |
| BK000964.3 | 826 | 1746 | 0.330546287 |
| BK000964.3 | 10796 | 1742 | 0.313363283 |
| BK000964.3 | 205 | 1737 | 0.139353489 |
| BK000964.3 | 3363 | 1651 | 0.055069513 |
| BK000964.3 | 256 | 1611 | 0.14231353 |
| BK000964.3 | 8350 | 1590 | 0.447229621 |
| BK000964.3 | 3703 | 1558 | 0.165082687 |
| BK000964.3 | 239 | 1549 | 0.114151191 |
| BK000964.3 | 10833 | 1544 | 0.154626897 |
| BK000964.3 | 5055 | 1539 | 0.039522586 |
| BK000964.3 | 12564 | 1529 | 0.301699313 |
| BK000964.3 | 1120 | 1505 | 0.070415253 |
| BK000964.3 | 11161 | 1454 | 0.334949432 |
| BK000964.3 | 10547 | 1451 | 0.321098991 |
| BK000964.3 | 3875 | 1403 | 0.360214119 |
| BK000964.3 | 13279 | 1403 | 0.416058579 |
| BK000964.3 | 7156 | 1319 | 0.627384354 |
| BK000964.3 | 6228 | 1293 | 0.555559734 |
| BK000964.3 | 637 | 1272 | −0.018872793 |
| BK000964.3 | 10270 | 1269 | 0.108764722 |
| BK000964.3 | 8884 | 1204 | 0.142932722 |
| BK000964.3 | 8577 | 1185 | 0.319122275 |
| BK000964.3 | 12702 | 1169 | 0.381800285 |
| BK000964.3 | 4551 | 1167 | 0.280366547 |
| BK000964.3 | 5903 | 1163 | 0.47303601 |
| BK000964.3 | 3712 | 1089 | 0.130507036 |
| BK000964.3 | 13246 | 1060 | 0.594944722 |
| BK000964.3 | 865 | 1021 | 0.263294293 |
| BK000964.3 | 9208 | 1004 | 0.228121154 |
| BK000964.3 | 13288 | 1001 | 0.463759872 |
| BK000964.3 | 12713 | 984 | 0.177162809 |
| BK000964.3 | 631 | 979 | 0.008583119 |
| BK000964.3 | 3517 | 959 | 0.303313576 |
| BK000964.3 | 11787 | 957 | 0.123006143 |
| BK000964.3 | 818 | 949 | 0.082018638 |
| BK000964.3 | 11411 | 914 | 0.314402246 |
| BK000964.3 | 6846 | 887 | 0.029773246 |
| BK000964.3 | 6105 | 877 | 0.567631465 |
| BK000964.3 | 7464 | 858 | 0.58283943 |
| BK000964.3 | 11776 | 857 | 0.173884234 |
| BK000964.3 | 12854 | 829 | 0.161926317 |
| BK000964.3 | 11794 | 821 | 0.233418044 |
| BK000964.3 | 3229 | 810 | 0.385755077 |
| BK000964.3 | 3724 | 774 | 0.172215983 |
| BK000964.3 | 10014 | 763 | 0.142174253 |
| BK000964.3 | 9421 | 744 | 0.057568096 |
| BK000964.3 | 9446 | 728 | 0.098193147 |
| BK000964.3 | 9341 | 706 | 0.175556936 |
| BK000964.3 | 10828 | 704 | 0.198354621 |
| BK000964.3 | 7370 | 700 | 0.596773772 |
| BK000964.3 | 7354 | 673 | 0.378240869 |
| BK000964.3 | 9126 | 658 | 0.233683816 |
| BK000964.3 | 4525 | 654 | 0.083651429 |
| BK000964.3 | 9184 | 651 | 0.153068009 |
| BK000964.3 | 932 | 645 | 0.434441372 |
| BK000964.3 | 6653 | 635 | 0.701133671 |
| BK000964.3 | 5763 | 631 | 0.38166856 |
| BK000964.3 | 3208 | 623 | 0.587452328 |
| BK000964.3 | 3297 | 622 | 0.204320009 |
| BK000964.3 | 9839 | 604 | 0.272645941 |
| BK000964.3 | 5133 | 604 | −0.088834787 |
| BK000964.3 | 10101 | 585 | 0.457505863 |
| BK000964.3 | 7802 | 577 | 0.106769547 |
| BK000964.3 | 741 | 570 | 0.129030262 |
| BK000964.3 | 12869 | 562 | 0.225698188 |
| BK000964.3 | 12589 | 562 | 0.403018626 |
| BK000964.3 | 4935 | 561 | 0.26327314 |
| BK000964.3 | 11380 | 528 | 0.276853452 |
| BK000964.3 | 5344 | 516 | 0.078318587 |
| BK000964.3 | 4377 | 498 | 0.147146249 |
| BK000964.3 | 2423 | 497 | 0.17854886 |
| BK000964.3 | 1472 | 491 | 0.327802712 |
| BK000964.3 | 5910 | 482 | 0.381492886 |
| BK000964.3 | 1087 | 479 | 0.160658702 |
| BK000964.3 | 1140 | 477 | −0.084218603 |
| BK000964.3 | 221 | 474 | 0.279269117 |
| BK000964.3 | 5171 | 474 | 0.300716492 |
| BK000964.3 | 2700 | 461 | 0.090628598 |
| BK000964.3 | 1173 | 456 | −0.140440968 |
| BK000964.3 | 12873 | 452 | 0.265687605 |
| BK000964.3 | 793 | 451 | 0.075509569 |
| BK000964.3 | 11764 | 446 | 0.01309535 |
| BK000964.3 | 7233 | 444 | 0.4662551 |
| BK000964.3 | 2492 | 443 | 0.284894104 |
| BK000964.3 | 9440 | 441 | 0.151153379 |
| BK000964.3 | 6836 | 440 | 0.069315968 |
| BK000964.3 | 13333 | 425 | 0.441610277 |
| BK000964.3 | 8770 | 419 | 0.301007296 |
| BK000964.3 | 7225 | 416 | 0.407016029 |
| BK000964.3 | 11875 | 412 | 0.003877922 |
| BK000964.3 | 2928 | 412 | 0.395044638 |
| BK000964.3 | 10574 | 411 | 0.467426363 |
| BK000964.3 | 12542 | 405 | 0.190219542 |
| BK000964.3 | 6374 | 402 | 0.28503482 |
| BK000964.3 | 4949 | 400 | 0.565470166 |
| BK000964.3 | 12578 | 398 | 0.2930323 |
| BK000964.3 | 11131 | 386 | 0.348366941 |
| BK000964.3 | 867 | 382 | 0.122761151 |
| BK000964.3 | 11094 | 379 | 0.248534569 |
| BK000964.3 | 12888 | 377 | 0.458319564 |
| BK000964.3 | 5581 | 376 | 0.400790205 |
| BK000964.3 | 12581 | 372 | 0.449857413 |
| BK000964.3 | 2968 | 369 | 0.230495563 |
| BK000964.3 | 1498 | 366 | 0.36789415 |
| BK000964.3 | 12743 | 362 | 0.436028787 |
| BK000964.3 | 7976 | 356 | 0.321208063 |
| BK000964.3 | 750 | 350 | 0.156380436 |
| BK000964.3 | 1041 | 346 | 0.326347862 |
| BK000964.3 | 5467 | 341 | −0.004779023 |
| BK000964.3 | 2498 | 330 | 0.22835543 |
| BK000964.3 | 11911 | 329 | 0.354854196 |
| BK000964.3 | 2672 | 329 | 0.121373422 |
| BK000964.3 | 5075 | 323 | 0.144169428 |
| BK000964.3 | 11781 | 323 | 0.159850597 |
| BK000964.3 | 4511 | 316 | 0.1749867 |
| BK000964.3 | 8461 | 314 | 0.372741972 |
| BK000964.3 | 2688 | 314 | 0.12068543 |
| BK000964.3 | 12554 | 314 | 0.36125755 |
| BK000964.3 | −398 | 311 | 0.235147056 |
| BK000964.3 | 12877 | 311 | 0.309015514 |
| BK000964.3 | 9286 | 310 | 0.094652509 |
| BK000964.3 | 10041 | 308 | 0.184153472 |
| BK000964.3 | 13361 | 304 | 0.448650911 |
| BK000964.3 | 5554 | 304 | 0.343027457 |
| BK000964.3 | 1022 | 296 | 0.423885255 |
| BK000964.3 | 3431 | 296 | 0.539771171 |
| BK000964.3 | 3879 | 296 | 0.383295088 |
| BK000964.3 | 703 | 295 | 0.162176343 |
| BK000964.3 | 639 | 294 | −0.019171531 |
| BK000964.3 | 7474 | 288 | 0.538244166 |
| BK000964.3 | 9986 | 283 | 0.148320869 |
| BK000964.3 | 8773 | 283 | 0.353822506 |
| BK000964.3 | 11152 | 281 | 0.333643926 |
| BK000964.3 | 10382 | 279 | 0.291233454 |
| BK000964.3 | 7817 | 278 | 0.21840873 |
| BK000964.3 | 10254 | 276 | 0.026293016 |
| BK000964.3 | 7179 | 272 | 0.509816699 |
| BK000964.3 | 6833 | 272 | 0.089588221 |
| BK000964.3 | 11715 | 268 | 0.256753771 |
| BK000964.3 | 10024 | 265 | 0.075172285 |
| BK000964.3 | 6094 | 256 | 0.292559096 |
| BK000964.3 | 7602 | 256 | 0.464959662 |
| BK000964.3 | 7561 | 249 | 0.586420341 |
| BK000964.3 | 4939 | 245 | 0.385095612 |
| BK000964.3 | 12595 | 244 | 0.28104051 |
| BK000964.3 | 1416 | 241 | 0.332373659 |
| BK000964.3 | 13328 | 233 | 0.42510986 |
| BK000964.3 | 6147 | 232 | 0.364601859 |
| BK000964.3 | 7482 | 228 | 0.586608281 |
| BK000964.3 | 7546 | 226 | 0.412261202 |
| BK000964.3 | 10049 | 225 | 0.064515131 |
| BK000964.3 | 10062 | 223 | 0.198632501 |
| BK000964.3 | 2439 | 217 | 0.260470172 |
| BK000964.3 | 12662 | 213 | 0.486727445 |
| BK000964.3 | 3883 | 211 | 0.221491267 |
| BK000964.3 | 3235 | 207 | 0.224353982 |
| BK000964.3 | 9121 | 207 | 0.036419913 |
| BK000964.3 | 6121 | 204 | 0.341758868 |
| BK000964.3 | 2693 | 203 | 0.144260042 |
| BK000964.3 | 7518 | 202 | 0.440044307 |
| BK000964.3 | 3873 | 200 | 0.280277846 |
| BK000964.3 | 11010 | 197 | 0.311931926 |
| BK000964.3 | 5579 | 197 | 0.270734668 |
| BK000964.3 | 12883 | 190 | 0.370604164 |
| BK000964.3 | 7488 | 189 | 0.655476216 |
| BK000964.3 | 9637 | 189 | −0.068287337 |
| BK000964.3 | 3313 | 185 | 0.306924691 |
| BK000964.3 | 9486 | 184 | 0.302644042 |
| BK000964.3 | 12717 | 183 | 0.33785242 |
| BK000964.3 | 2431 | 181 | 0.384837195 |
| BK000964.3 | 2974 | 178 | 0.290100785 |
| BK000964.3 | 2255 | 177 | 0.642558758 |
| BK000964.3 | 3537 | 175 | 0.103750775 |
| BK000964.3 | 1593 | 174 | 0.003726903 |
| BK000964.3 | 9366 | 173 | 0.180461811 |
| BK000964.3 | 4287 | 168 | 0.256075418 |
| BK000964.3 | 4956 | 167 | 0.246265668 |
| BK000964.3 | 12861 | 167 | 0.189655045 |
| BK000964.3 | 717 | 164 | 0.26843577 |
| BK000964.3 | 12844 | 160 | 0.228396542 |
| BK000964.3 | 12685 | 157 | 0.276528897 |
| BK000964.3 | 9190 | 157 | 0.073220739 |
| BK000964.3 | 8758 | 156 | 0.297826674 |
| BK000964.3 | 9664 | 154 | 0.590059984 |
| BK000964.3 | 11091 | 153 | 0.196189965 |
| BK000964.3 | 9895 | 152 | 0.343470456 |
| BK000964.3 | 5261 | 148 | 0.213232014 |
| BK000964.3 | 10083 | 146 | −0.029999779 |
| BK000964.3 | 8867 | 141 | 0.401545317 |
| BK000964.3 | 5286 | 141 | 0.377346796 |
| BK000964.3 | 1112 | 140 | 0.207080712 |
| BK000964.3 | 10606 | 139 | 0.301919135 |
| BK000964.3 | 11113 | 137 | 0.242523869 |
| BK000964.3 | 1720 | 137 | 0.101577729 |
| BK000964.3 | 6969 | 137 | 0.107091728 |
| BK000964.3 | 6854 | 137 | 0.472629927 |
| BK000964.3 | 1705 | 136 | 0.237249625 |
| BK000964.3 | 5213 | 135 | 0.090101698 |
| BK000964.3 | 7123 | 135 | 0.521286018 |
| BK000964.3 | 2883 | 134 | 0.256630845 |
| BK000964.3 | 13235 | 132 | 0.314230824 |
| BK000964.3 | 8781 | 131 | 0.371577421 |
| BK000964.3 | 1755 | 130 | 0.365647271 |
| BK000964.3 | 9301 | 129 | 0.173534913 |
| BK000964.3 | 967 | 127 | 0.284145465 |
| BK000964.3 | 12865 | 126 | 0.213643131 |
| BK000964.3 | 11100 | 126 | 0.222472913 |
| BK000964.3 | 1639 | 120 | 0.280161827 |
| BK000964.3 | 2887 | 119 | 0.270201055 |
| BK000964.3 | 7849 | 118 | 0.29537147 |
| BK000964.3 | 1341 | 118 | 0.186088238 |
| BK000964.3 | 10029 | 116 | 0.097917951 |
| BK000964.3 | 3758 | 116 | 0.485319172 |
| BK000964.3 | 13241 | 115 | 0.458945611 |
| BK000964.3 | 1747 | 115 | 0.293364549 |
| BK000964.3 | 10212 | 114 | 0.090053035 |
| BK000964.3 | 7819 | 114 | 0.282568119 |
| BK000964.3 | 5520 | 113 | 0.421023863 |
| BK000964.3 | 10813 | 113 | 0.247294299 |
| BK000964.3 | 7582 | 112 | 0.640191061 |
| BK000964.3 | 3575 | 112 | 0.334868886 |
| BK000964.3 | 5569 | 111 | 0.490920429 |
| BK000964.3 | 2463 | 110 | 0.378992625 |
| BK000964.3 | 5241 | 110 | 0.45928244 |
| BK000964.3 | 2724 | 109 | 0.207649216 |
| BK000964.3 | 3817 | 108 | 0.23925319 |
| BK000964.3 | 11895 | 107 | 0.001496801 |
| BK000964.3 | 1628 | 107 | −0.003562453 |
| BK000964.3 | 3305 | 106 | 0.299172203 |
| BK000964.3 | 10056 | 106 | 0.04848749 |
| BK000964.3 | 5532 | 105 | 0.458405286 |
| BK000964.3 | 7452 | 104 | 0.519532479 |
| BK000964.3 | 710 | 104 | 0.255743168 |
| BK000964.3 | 1565 | 104 | 0.226120945 |
| BK000964.3 | 3848 | 102 | 0.334546705 |
| BK000964.3 | 7230 | 100 | 0.436483532 |
| BK000964.3 | 2336 | 100 | 0.496280049 |
| BK000964.3 | −166 | 100 | 0.206315184 |
| BK000964.3 | 11103 | 100 | 0.317502978 |
| BK000964.3 | 11886 | 98 | 0.026692387 |
| BK000964.3 | 11149 | 96 | 0.488525883 |
| BK000964.3 | 12731 | 96 | 0.239209123 |
| BK000964.3 | 9312 | 96 | 0.335882415 |
| BK000964.3 | 9640 | 96 | −0.048001701 |
| BK000964.3 | 9199 | 95 | 0.271397489 |
| BK000964.3 | 2970 | 95 | 0.249903632 |
| BK000964.3 | 11007 | 94 | 0.57736906 |
| BK000964.3 | 8472 | 94 | 0.426168696 |
| BK000964.3 | −218 | 94 | 0.144466439 |
| BK000964.3 | 5754 | 93 | 0.222886733 |
| BK000964.3 | 7530 | 93 | 0.311284207 |
| BK000964.3 | 786 | 91 | 0.277621293 |
| BK000964.3 | 11002 | 91 | 0.616228826 |
| BK000964.3 | 9345 | 90 | 0.340221795 |
| BK000964.3 | 2232 | 89 | 0.261190046 |
| BK000964.3 | 2339 | 88 | 0.553995812 |
| BK000964.3 | 1491 | 87 | 0.353441593 |
| BK000964.3 | 7109 | 85 | 0.166621439 |
| BK000964.3 | 12592 | 84 | 0.520431902 |
| BK000964.3 | 9225 | 82 | 0.144731567 |
| BK000964.3 | 10386 | 82 | 0.564876144 |
| BK000964.3 | 5114 | 82 | 0.244339292 |
| BK000964.3 | 7198 | 81 | 0.321206385 |
| BK000964.3 | 12597 | 80 | 0.347692665 |
| BK000964.3 | 9631 | 80 | −0.020188351 |
| BK000964.3 | 12633 | 80 | 0.424054736 |
| BK000964.3 | 6989 | 80 | 0.110076939 |
| BK000964.3 | 5712 | 80 | 0.177516873 |
| BK000964.3 | 9111 | 79 | 0.193570564 |
| BK000964.3 | 4186 | 78 | 0.218853407 |
| BK000964.3 | 3524 | 75 | 0.296317878 |
| BK000964.3 | 5093 | 75 | 0.452058985 |
| BK000964.3 | 12567 | 73 | 0.34607085 |
| BK000964.3 | 11097 | 73 | 0.262728797 |
| BK000964.3 | 12709 | 73 | 0.20967963 |
| BK000964.3 | 13374 | 72 | 0.442281118 |
| BK000964.3 | 4080 | 72 | 0.434129259 |
| BK000964.3 | 4221 | 72 | 0.037242147 |
| BK000964.3 | 4096 | 71 | 0.494831911 |
| BK000964.3 | 12587 | 70 | 0.340163064 |
| BK000964.3 | 3222 | 68 | 0.268996231 |
| BK000964.3 | 9065 | 66 | 0.291758677 |
| BK000964.3 | 5534 | 65 | 0.414123813 |
| BK000964.3 | 1699 | 65 | 0.206571922 |
| BK000964.3 | 5083 | 65 | 0.06081508 |
| BK000964.3 | 6864 | 64 | 0.214255611 |
| BK000964.3 | 2900 | 64 | 0.463575289 |
| BK000964.3 | 9305 | 62 | 0.294074355 |
| BK000964.3 | 7995 | 62 | 0.331559816 |
| BK000964.3 | 8300 | 62 | 0.209501759 |
| BK000964.3 | 6226 | 61 | 0.490576433 |
| BK000964.3 | 5785 | 61 | 0.37857983 |
| BK000964.3 | 7540 | 59 | 0.503876481 |
| BK000964.3 | 13343 | 58 | 0.397581816 |
| BK000964.3 | 3455 | 58 | 0.246649936 |
| BK000964.3 | 7058 | 58 | 0.717987782 |
| BK000964.3 | 8430 | 57 | 0.283447406 |
| BK000964.3 | 7014 | 57 | 0.639496358 |
| BK000964.3 | 4209 | 55 | 0.132097697 |
| BK000964.3 | 3308 | 55 | 0.195085823 |
| BK000964.3 | 7979 | 54 | 0.477336795 |
| BK000964.3 | 6810 | 54 | 0.280792766 |
| BK000964.3 | 6082 | 52 | 0.354902859 |
| BK000964.3 | 7809 | 52 | 0.05485137 |
| BK000964.3 | 7992 | 52 | 0.220147166 |
| BK000964.3 | 7245 | 51 | 0.276983643 |
| BK000964.3 | 2840 | 51 | 0.189964482 |
| BK000964.3 | 7814 | 51 | 0.115797336 |
| BK000964.3 | 2903 | 50 | 0.429051547 |
| BK000964.3 | 13253 | 50 | 0.452085834 |
| BK000964.3 | 6216 | 50 | 0.403540492 |
| BK000964.3 | 4136 | 49 | 0.241344012 |
| BK000964.3 | 11915 | 48 | 0.191100507 |
| BK000964.3 | 12879 | 48 | 0.402530657 |
| BK000964.3 | 989 | 48 | 0.21904638 |
| BK000964.3 | 8913 | 48 | 0.450008435 |
| BK000964.3 | 12560 | 47 | 0.435209909 |
| BK000964.3 | 3508 | 46 | 0.253888948 |
| BK000964.3 | 4985 | 46 | −0.010442366 |
| BK000964.3 | 3547 | 46 | 0.11946047 |
| BK000964.3 | 2242 | 46 | 0.446726213 |
| BK000964.3 | 9054 | 45 | 0.279864816 |
| BK000964.3 | 4345 | 45 | 0.208966468 |
| BK000964.3 | 5805 | 45 | 0.17530691 |
| BK000964.3 | 10105 | 43 | 0.007693757 |
| BK000964.3 | 10794 | 43 | 0.216525983 |
| BK000964.3 | 1718 | 42 | 0.054426874 |
| BK000964.3 | 12646 | 41 | 0.326191805 |
| BK000964.3 | 3607 | 41 | 0.201504279 |
| BK000964.3 | 12638 | 41 | 0.51492169 |
| BK000964.3 | 13271 | 41 | 0.225706472 |
| BK000964.3 | 1607 | 39 | 0.187850167 |
| BK000964.3 | 2388 | 39 | 0.019377864 |
| BK000964.3 | 10035 | 37 | 0.121583175 |
| BK000964.3 | 2263 | 37 | 0.540224239 |
| BK000964.3 | 10103 | 37 | 0.170262759 |
| BK000964.3 | 7536 | 37 | 0.351622315 |
| BK000964.3 | 6630 | 37 | 0.388396292 |
| BK000964.3 | 7114 | 37 | 0.235074901 |
| BK000964.3 | 5590 | 36 | 0.492788074 |
| BK000964.3 | 9049 | 36 | 0.17830219 |
| BK000964.3 | 7166 | 35 | 0.488896727 |
| BK000964.3 | 6320 | 35 | 0.457893488 |
| BK000964.3 | 13308 | 34 | 0.394657014 |
| BK000964.3 | 3532 | 34 | 0.341648118 |
| BK000964.3 | 1739 | 34 | 0.254565193 |
| BK000964.3 | 13336 | 34 | 0.351855561 |
| BK000964.3 | 10579 | 34 | 0.366236258 |
| BK000964.3 | 2326 | 34 | 0.534299123 |
| BK000964.3 | 6219 | 33 | 0.387011919 |
| BK000964.3 | 230 | 33 | 0.138346672 |
| BK000964.3 | 5280 | 33 | 0.250067449 |
| BK000964.3 | 7460 | 33 | 0.50732315 |
| BK000964.3 | 9320 | 32 | 0.163114361 |
| BK000964.3 | 3866 | 32 | 0.294720396 |
| BK000964.3 | 3376 | 31 | 0.180935015 |
| BK000964.3 | 2483 | 31 | 0.479885719 |
| BK000964.3 | 3591 | 31 | 0.163439898 |
| BK000964.3 | 7943 | 31 | 0.326666961 |
| BK000964.3 | 3248 | 31 | 0.328156776 |
| BK000964.3 | 3871 | 31 | 0.331543036 |
| BK000964.3 | 2197 | 31 | 0.560452863 |
| BK000964.3 | 7141 | 29 | 0.372824195 |
| BK000964.3 | 8019 | 29 | 0.40076 |
| BK000964.3 | 12673 | 29 | 0.224640925 |
| BK000964.3 | 9092 | 29 | 0.311403347 |
| BK000964.3 | 5776 | 29 | 0.202172134 |
| BK000964.3 | 13267 | 28 | 0.474928825 |
| BK000964.3 | 1375 | 28 | 0.318370518 |
| BK000964.3 | 2972 | 28 | 0.303266591 |
| BK000964.3 | 12891 | 28 | 0.276830942 |
| BK000964.3 | 3485 | 27 | 0.453223536 |
| BK000964.3 | 1576 | 27 | 0.074247692 |
| BK000964.3 | 7394 | 27 | 0.60134472 |
| BK000964.3 | 1505 | 26 | 0.355334112 |
| BK000964.3 | 8024 | 26 | 0.387970073 |
| BK000964.3 | 3318 | 26 | 0.213792476 |
| BK000964.3 | 10650 | 26 | 0.36101927 |
| BK000964.3 | 6297 | 26 | 0.178538792 |
| BK000964.3 | 10070 | 25 | 0.110744794 |
| BK000964.3 | −220 | 25 | −0.000885999 |
| BK000964.3 | 1475 | 25 | 0.385580562 |
| BK000964.3 | −196 | 25 | 0.484040515 |
| BK000964.3 | 10742 | 25 | 0.150962084 |
| BK000964.3 | 846 | 25 | 0.192307009 |
| BK000964.3 | 5530 | 25 | 0.420501996 |
| BK000964.3 | 10090 | 25 | 0.064060386 |
| BK000964.3 | 3813 | 24 | 0.329435433 |
| BK000964.3 | 7427 | 24 | 0.473536062 |
| BK000964.3 | 673 | 24 | 0.427000998 |
| BK000964.3 | 5040 | 23 | 0.011700887 |
| BK000964.3 | 6637 | 23 | 0.298240898 |
| BK000964.3 | 2780 | 22 | 0.363910512 |
| BK000964.3 | 5072 | 22 | 0.112132523 |
| BK000964.3 | 973 | 22 | 0.269675832 |
| BK000964.3 | 4342 | 22 | 0.269977877 |
| BK000964.3 | 9385 | 22 | 0.336565372 |
| BK000964.3 | 5749 | 22 | 0.260349354 |
| BK000964.3 | 10039 | 22 | 0.118755798 |
| BK000964.3 | 3742 | 21 | 0.287280021 |
| BK000964.3 | 1006 | 21 | 0.351236369 |
| BK000964.3 | 7345 | 20 | 0.323679798 |
| BK000964.3 | 7858 | 20 | 0.325262178 |
| BK000964.3 | 7467 | 20 | 0.45123843 |
| BK000964.3 | 10075 | 20 | 0.00700241 |
| BK000964.3 | 5879 | 20 | 0.14746004 |
| BK000964.3 | 10347 | 20 | 0.361551205 |
| BK000964.3 | 12576 | 19 | 0.330818128 |
| BK000964.3 | 1004 | 19 | 0.407545944 |
| BK000964.3 | 7200 | 19 | 0.418792086 |
| BK000964.3 | 7412 | 19 | 0.476172243 |
| BK000964.3 | 3382 | 19 | 0.151930306 |
| BK000964.3 | 13363 | 19 | 0.40716671 |
| BK000964.3 | 7557 | 19 | 0.582671628 |
| BK000964.3 | 8397 | 19 | 0.334439311 |
| BK000964.3 | 7208 | 18 | 0.501926613 |
| BK000964.3 | 2319 | 18 | 0.511570238 |
| BK000964.3 | 3398 | 18 | 0.34499075 |
| BK000964.3 | 9212 | 18 | 0.289129207 |
| BK000964.3 | 9644 | 18 | 0.214415203 |
| BK000964.3 | 6202 | 18 | 0.400701269 |
| BK000964.3 | 12610 | 18 | 0.283727636 |
| BK000964.3 | 3730 | 17 | 0.28262685 |
| BK000964.3 | 10491 | 17 | 0.260193298 |
| BK000964.3 | 761 | 17 | 0.124588523 |
| BK000964.3 | 3343 | 16 | 0.187157142 |
| BK000964.3 | 9625 | 16 | 0.021745561 |
| BK000964.3 | 9172 | 16 | 0.19610103 |
| BK000964.3 | 7088 | 16 | 0.300878757 |
| BK000964.3 | 9202 | 15 | 0.285878868 |
| BK000964.3 | 11906 | 15 | 0.38052802 |
| BK000964.3 | 9825 | 15 | 0.337117444 |
| BK000964.3 | 5324 | 14 | 0.154165439 |
| BK000964.3 | 1351 | 14 | 0.484520431 |
| BK000964.3 | 10780 | 14 | 0.506853302 |
| BK000964.3 | 7895 | 14 | 0.380663941 |
| BK000964.3 | 9845 | 14 | 0.240869131 |
| BK000964.3 | 9104 | 14 | 0.234138561 |
| BK000964.3 | 6241 | 14 | 0.269456011 |
| BK000964.3 | 9601 | 14 | 0.118550979 |
| BK000964.3 | 2911 | 14 | 0.423728844 |
| BK000964.3 | 6259 | 13 | 0.160939637 |
| BK000964.3 | 8893 | 13 | 0.307307281 |
| BK000964.3 | 11868 | 13 | −0.001614263 |
| BK000964.3 | 6184 | 13 | 0.266536242 |
| BK000964.3 | −247 | 13 | 0.388347629 |
| BK000964.3 | 9382 | 13 | 0.423754014 |
| BK000964.3 | 8403 | 13 | 0.324367789 |
| BK000964.3 | 4193 | 12 | 0.102504 |
| BK000964.3 | 5604 | 12 | 0.375535888 |
| BK000964.3 | 7826 | 12 | 0.253054968 |
| BK000964.3 | 7170 | 12 | 0.508264523 |
| BK000964.3 | 13292 | 12 | 0.199200347 |
| BK000964.3 | 2228 | 12 | 0.236314963 |
| BK000964.3 | 10526 | 11 | 0.172231085 |
| BK000964.3 | 6224 | 11 | 0.429046513 |
| BK000964.3 | 8344 | 11 | 0.189254676 |
| BK000964.3 | 12550 | 11 | 0.479475003 |
| BK000964.3 | 2746 | 10 | 0.423087837 |
| BK000964.3 | 13339 | 10 | 0.351716285 |
| BK000964.3 | 1025 | 10 | 0.36829352 |
| BK000964.3 | 10851 | 10 | 0.155804872 |
| BK000964.3 | 6208 | 10 | 0.356428186 |
| BK000964.3 | 9181 | 10 | 0.207060228 |
| BK000964.3 | 2735 | 10 | 0.232257492 |
| BK000964.3 | 8896 | 10 | 0.22532556 |
| BK000964.3 | 3697 | 10 | 0.190284985 |
| BK000964.3 | 6176 | 10 | 0.34892069 |
| BK000964.3 | 6644 | 10 | 0.534451823 |
| BK000964.3 | 7006 | 10 | 0.333771456 |
| BK000964.3 | 2896 | 10 | 0.41951196 |
| BK000964.3 | 10047 | 10 | 0.090276212 |
| BK000964.3 | 7882 | 10 | 0.235920627 |
| BK000964.3 | 5708 | 9 | 0.25582225 |
| BK000964.3 | 7206 | 9 | 0.569061145 |
| BK000964.3 | 4189 | 9 | 0.128093923 |
| BK000964.3 | 3589 | 9 | 0.225030227 |
| BK000964.3 | 4334 | 9 | 0.236155551 |
| BK000964.3 | 7864 | 9 | 0.428840116 |
| BK000964.3 | 1373 | 8 | 0.293621288 |
| BK000964.3 | 7149 | 8 | 0.406777408 |
| BK000964.3 | 10820 | 8 | 0.288696276 |
| BK000964.3 | 3827 | 8 | 0.440903457 |
| BK000964.3 | 2729 | 7 | 0.279455378 |
| BK000964.3 | 895 | 7 | 0.367848843 |
| BK000964.3 | 7555 | 7 | 0.487052575 |
| BK000964.3 | 6257 | 7 | 0.277732043 |
| BK000964.3 | 1430 | 7 | 0.311364752 |
| BK000964.3 | 5769 | 7 | 0.215289276 |
| BK000964.3 | 7408 | 7 | 0.498213137 |
| BK000964.3 | 8543 | 6 | 0.418968279 |
| BK000964.3 | 2300 | 6 | 0.462117083 |
| BK000964.3 | 6197 | 6 | 0.238078571 |
| BK000964.3 | 7441 | 6 | 0.419147828 |
| BK000964.3 | 3348 | 6 | 0.1382376 |
| BK000964.3 | 6977 | 6 | 0.396994506 |
| BK000964.3 | 7285 | 6 | 0.332054834 |
| BK000964.3 | 7544 | 6 | 0.509397192 |
| BK000964.3 | 8928 | 6 | 0.302570209 |
| BK000964.3 | 10359 | 6 | 0.216529339 |
| BK000964.3 | 4132 | 6 | 0.377625032 |
| BK000964.3 | 2474 | 6 | 0.414110389 |
| BK000964.3 | 5642 | 6 | 0.213750525 |
| BK000964.3 | 2212 | 6 | 0.30528358 |
| BK000964.3 | 8303 | 6 | 0.405681656 |
| BK000964.3 | 11120 | 6 | 0.521132076 |
| BK000964.3 | 9835 | 6 | 0.360666884 |
| BK000964.3 | 7239 | 6 | 0.387911342 |
| BK000964.3 | 3466 | 6 | 0.263029164 |
| BK000964.3 | 2331 | 5 | 0.498276902 |
| BK000964.3 | 5715 | 5 | 0.169464018 |
| BK000964.3 | 5760 | 5 | 0.277347775 |
| BK000964.3 | 4327 | 5 | 0.059801551 |
| BK000964.3 | 7385 | 5 | 0.434501781 |
| BK000964.3 | 7212 | 5 | 0.446597005 |
| BK000964.3 | 9975 | 5 | 0.253202634 |
| BK000964.3 | 9068 | 5 | 0.283829996 |
| BK000964.3 | 8908 | 5 | 0.393881765 |
| BK000964.3 | 10215 | 5 | 0.229799182 |
| BK000964.3 | 4323 | 5 | 0.124210967 |
| BK000964.3 | 6127 | 5 | 0.374906627 |
| BK000964.3 | 10202 | 5 | 0.118302631 |
| BK000964.3 | 5936 | 5 | 0.281453909 |
| BK000964.3 | 7130 | 5 | 0.3503252 |
| BK000964.3 | 2316 | 5 | 0.571539592 |
| BK000964.3 | 11159 | 5 | 0.458464017 |
| BK000964.3 | −237 | 5 | 0.232222254 |
| BK000964.3 | 8307 | 4 | 0.381769761 |
| BK000964.3 | 834 | 4 | 0.216975694 |
| BK000964.3 | 11934 | 4 | 0.210652886 |
| BK000964.3 | 9228 | 4 | 0.233912028 |
| BK000964.3 | 6347 | 4 | 0.398598195 |
| BK000964.3 | 2308 | 4 | 0.368023358 |
| BK000964.3 | 1432 | 4 | 0.367868448 |
| BK000964.3 | 10815 | 4 | 0.284747877 |
| BK000964.3 | 2737 | 4 | 0.250558063 |
| BK000964.3 | 6639 | 4 | 0.267287999 |
| BK000964.3 | 6107 | 4 | 0.533429905 |
| BK000964.3 | 5835 | 4 | 0.252054235 |
| BK000964.3 | 2953 | 4 | 0.361314603 |
| BK000964.3 | 10736 | 4 | 0.283115156 |
| BK000964.3 | 7181 | 4 | 0.257436298 |
| BK000964.3 | 9391 | 4 | 0.338154465 |
| BK000964.3 | 10818 | 4 | 0.481447962 |
| BK000964.3 | 7608 | 4 | 0.301660718 |
| BK000964.3 | 1436 | 4 | 0.297621706 |
| BK000964.3 | 6796 | 4 | 0.261827697 |
| BK000964.3 | 844 | 4 | 0.227127762 |
| BK000964.3 | 7249 | 4 | 0.431983062 |
| BK000964.3 | 2870 | 3 | 0.217891897 |
| BK000964.3 | 8910 | 3 | 0.29646051 |
| BK000964.3 | 2741 | 3 | 0.381406787 |
| BK000964.3 | 2495 | 3 | 0.458203923 |
| BK000964.3 | 4387 | 3 | 0.208129132 |
| BK000964.3 | 10397 | 3 | 0.28010813 |
| BK000964.3 | 6817 | 3 | −0.019124482 |
| BK000964.3 | 13207 | 3 | 0.423853018 |
| BK000964.3 | 10542 | 3 | 0.288979863 |
| BK000964.3 | 9282 | 3 | 0.15409664 |
| BK000964.3 | 3385 | 3 | 0.471203603 |
| BK000964.3 | 5047 | 3 | −0.014380697 |
| BK000964.3 | 6212 | 3 | 0.489160178 |
| BK000964.3 | 2913 | 3 | 0.453389661 |
| BK000964.3 | 737 | 3 | 0.136495807 |
| BK000964.3 | 6254 | 3 | 0.26361144 |
| BK000964.3 | −260 | 3 | 0.338909577 |
| BK000964.3 | 8898 | 3 | 0.393638451 |
| BK000964.3 | 9074 | 3 | 0.396517946 |
| BK000964.3 | 3488 | 3 | 0.332524682 |
| BK000964.3 | 2799 | 3 | 0.282472471 |
| BK000964.3 | 7443 | 3 | 0.434867591 |
| BK000964.3 | 7350 | 3 | 0.180408114 |
| BK000964.3 | 3514 | 3 | 0.336603967 |
| BK000964.3 | 7174 | 3 | 0.433790298 |
| BK000964.3 | 10784 | 3 | 0.22496814 |
| BK000964.3 | 2200 | 3 | 0.260156381 |
| BK000964.3 | 2281 | 2 | 0.553072897 |
| BK000964.3 | 3771 | 2 | 0.354003436 |
| BK000964.3 | 6330 | 2 | 0.45031048 |
| BK000964.3 | 9070 | 2 | 0.28069544 |
| BK000964.3 | 1442 | 2 | 0.322287035 |
| BK000964.3 | 13244 | 2 | 0.513510038 |
| BK000964.3 | 4982 | 2 | 0.26590027 |
| BK000964.3 | 6361 | 2 | 0.472789339 |
| BK000964.3 | 11852 | 2 | 0.2769182 |
| BK000964.3 | 3803 | 2 | 0.334373868 |
| BK000964.3 | 7364 | 2 | 0.366370501 |
| BK000964.3 | 730 | 2 | 0.266725859 |
| BK000964.3 | 2758 | 2 | 0.456718868 |
| BK000964.3 | 5801 | 2 | 0.415674311 |
| BK000964.3 | 2205 | 2 | 0.453221858 |
| BK000964.3 | 5897 | 2 | 0.338392745 |
| BK000964.3 | 6295 | 2 | 0.389815904 |
| BK000964.3 | 11024 | 2 | 0.318830298 |
| BK000964.3 | 3448 | 2 | 0.500847221 |
| BK000964.3 | 1428 | 2 | 0.302409119 |
| BK000964.3 | 9095 | 2 | 0.220283087 |
| BK000964.3 | 8297 | 2 | 0.481944659 |
| BK000964.3 | 9848 | 2 | 0.167302718 |
| BK000964.3 | 6621 | 2 | 0.455485518 |
| BK000964.3 | 5724 | 2 | 0.36861738 |
| BK000964.3 | 891 | 2 | 0.233422044 |
| BK000964.3 | 842 | 1 | 0.278119668 |
| BK000964.3 | 5002 | 1 | 0.484629503 |
| BK000964.3 | 7214 | 1 | 0.453277233 |
| BK000964.3 | 11923 | 1 | 0.220130386 |
| BK000964.3 | 3478 | 1 | 0.449109389 |
| BK000964.3 | 3811 | 1 | 0.310369682 |
| BK000964.3 | 9280 | 1 | 0.17095914 |
| BK000964.3 | 13323 | 1 | 0.323800616 |
| BK000964.3 | 12669 | 1 | 0.3651685 |
| BK000964.3 | 4354 | 1 | 0.295708754 |
| BK000964.3 | 6276 | 1 | 0.48891183 |
| BK000964.3 | 6334 | 1 | 0.467303867 |
| BK000964.3 | 6363 | 1 | 0.331920592 |
| BK000964.3 | 9353 | 1 | 0.279369798 |
| BK000964.3 | 7029 | 1 | 0.60767424 |
| BK000964.3 | 8466 | 1 | 0.170563126 |
| BK000964.3 | 8489 | 1 | 0.300507913 |
| BK000964.3 | 7053 | 1 | 0.557922397 |
| BK000964.3 | 4410 | 1 | 0.111642539 |
| BK000964.3 | 8332 | 1 | 0.229672683 |
| BK000964.3 | 9288 | 1 | 0.194813982 |
| BK000964.3 | 10750 | 1 | 0.232559537 |
| BK000964.3 | 4319 | 1 | 0.122595026 |
| BK000964.3 | 1440 | 1 | 0.312723955 |
| BK000964.3 | 8535 | 1 | 0.255645843 |
| BK000964.3 | 7311 | 1 | 0.29948096 |
| BK000964.3 | 3403 | 1 | 0.485188286 |
| BK000964.3 | 4339 | 1 | 0.17345269 |
| BK000964.3 | 10801 | 1 | 0.29521038 |
| BK000964.3 | 7036 | 1 | 0.573917357 |
| BK000964.3 | 4290 | 1 | 0.110033311 |
| BK000964.3 | 7437 | 1 | 0.547869333 |
| BK000964.3 | 13347 | 1 | 0.348846857 |
| BK000964.3 | 7890 | 1 | 0.200264216 |
| BK000964.3 | 6981 | 1 | 0.21963369 |
| BK000964.3 | 9222 | 1 | 0.276604409 |

In one embodiment, the set of methylation markers consists of, consists essentially of, or comprises 100% of the methylation sites selected from the sites listed in Table 8. In one embodiment, the set of methylation markers consists of, consists essentially of, or comprises at least 50% of the methylation sites selected from the sites listed in Table 8. In one embodiment, the set of methylation markers consists of, consists essentially of, or comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 8. One skilled in the art can determine if a biological sample is methylated at a methylation site listed in Table 8, e.g., using methods further described herein below.

In one embodiment, the methylation site of Table 1, Table 2, Table 5, Table 6, Table 7, or Table 8 is a methylation site of the human genome. In another embodiment, the methylation site of Table 1, Table 2, Table 5, Table 6, Table 7, or Table 8 a methylation site of a mammal genome, e.g., a mouse genome. Methylation sites that correlate with other species, for example, the correlative human methylation site of a mouse methylation site, can be used in the methylation clocks described herein. For example, if the methylation site in a given Table is a mouse methylation site, the correlative human methylation site can be used in its place. Table 3 presented herein shows exemplary methylations sites which correlate between the human and mouse genomes. One skilled in the art can identify a methylation site of another species that correlates with a methylation site presented in Table 1, Table 2, Table 5, Table 6, Table 7, or Table 8, for example, using prediction software, such as ClustalW (Thompson et al. 1994), available on the world wide web at www.genome.jp/tools-bin/clustalw, to align the sequences of pairs of species. Homologous CpG sites can be identified, e.g., by applying the Perl module Bio::AlignIO. To remove potential error due to misalignment, the sites can be further filtered by requiring that the two flanking nucleotides (immediately upstream and downstream of each focal CpG) also be identical between the pair of species.

In one embodiment, the methylation sites used in the model described herein are the same species as the subject whose methylation age is being measured. For example, human methylation sites are used in the model which measure the methylation age of a human. In an alternate embodiment, the methylation sites used in the model are a different species than that of the subject whose methylation age is being measured. For example, mouse methylation sites are used in the model which measure the methylation age of a human. Data presented herein show that the models presented herein effectively measure the methylation age across species.

Methods for DNA methylation analysis are divided into two types, e.g., global and gene-specific methylation analysis. For global methylation analysis, methods include measuring the overall level of methyl cytosines in genome, e.g., chromatographic methods and methyl accepting capacity assay. For gene-specific methylation analysis, a large number of techniques have been developed. Techniques include, e.g., methylation sensitive restriction enzymes to digest DNA followed by Southern detection or PCR amplification. Alternative techniques include bisulfite reaction based methods, such as methylation specific PCR (MSP), and bisulfite genomic sequencing PCR. Additionally, to identify unknown methylation hot-spots, e.g., methylated CpG islands in the genome, genome-wide screen methods are used, such as Restriction Landmark Genomic Scanning for Methylation (RLGS-M), and CpG island microarray.

Methods for identifying methylation markers are further reviewed in, e.g., Forat S., et al. PLoS ONE. January 2016; 11(2); Schatz, P., et al. Nucleic Acid Research. January 2006; 34(8): e59; Yi, S. H., et al. Forensic Science International Genetics. March 2014, which are incorporated herein by reference in their entireties. Various methods known in the art may be used for determining the methylation status of specific CpG dinucleotides. Such methods include but are not limited to, restriction landmark genomic scanning, see Kawai et al., “Comparison of DNA methylation patterns among mouse cell lines by restriction landmark genomic scanning,” Mol. Cell Biol. 14(11): 7421-7427 (1994); methylated CpG island amplification, see Toyota et al., “Identification of differentially methylated sequences in colorectal cancer by methylated CpG island amplification,” Cancer Res., 59: 2307-2312 (1999), see also WO00/26401A1; differential methylation hybridization, see Huang et al., “Methylation profiling of CpG islands in human breast cancer cells,” Hum. Mol. Genet., 8: 459-470 (1999); methylation-specific PCR (MSP), see Herman et al., “Methylation-specific PCR: a novel PCR assay for methylation status of CpG islands,” PNAS USA 93: 9821-9826 (1992), see also U.S. Pat. No. 5,786,146; methylation-sensitive single nucleotide primer extension (Ms-SnuPE), see U.S. Pat. No. 6,251,594; combined bisulfite restriction analysis (COBRA), see Xiong and Laird, “COBRA: a sensitive and quantitative DNA methylation assay,” Nucleic Acids Research, 25(12): 2532-2534 (1997); bisulfite genomic sequencing, see Frommer et al., “A genomic sequencing protocol that yields a positive display of 5-methylcytosine residues in individual DNA strands,” PNAS USA, 89: 1827-1831 (1992); and methylation-specific primer extension (MSPE), etc.

### Algorithm of Present Molecular Clock

In one embodiment of any aspect, the statistical prediction statistical prediction algorithm comprises: (a) identifying at least two coefficients found in Table 1, Table 2, Table 5, Table 6, Table 7, or Table 8 in a biological sample; (b) multiplying each of the at least two coefficients with its corresponding CpG's methylation level to output a value for each of the at least two coefficients; (c) find a sum of values of (b) for each identified coefficient; (d) adding a recalibration intercept to the summed values of (c); and (e) calculating the natural exponentiation of (d), wherein the exponentiation is the predicted methylation age of the subject.

### Biological Sample

DNA methylation age is a valuable biomarker for studying human development, aging, and cancer and can be used as a surrogate marker for evaluating rejuvenation therapies. The most salient feature of DNA methylation age is its applicability to a broad spectrum of tissues and cell types. DNA methylation age has been found to accurately predict age in various sources of DNA, including, but not limited to whole blood, adipose tissue/fat, blood (whole blood, cord blood, blood cells, peripheral blood mononuclear cells, B cells, T cells, monocytes), brain tissue (frontal cortex, temporal cortex, PONS), breast, buccal cells/epithelium, cartilage, cerebellum, colon, cortex (pre-frontal-, frontal-, occipital-, temporal cortex), epidermis, fibroblasts (e.g. dermal fibroblasts), gastric tissue, glial cells, head/neck tissue, kidney, lung, liver, mesenchymal stromal cells, neurons, pancreas, pons, prostate, saliva, heart tissue, stomach, thyroid, uterine cervix, and many other tissues/cell types. Furthermore, DNA methylation age of easily accessible fluids/tissues (e.g. saliva, buccal cells, blood, skin) can serve as a surrogate marker for inaccessible tissues (e.g. brain, kidney, liver). Further, DNA methylation age can be used to compare the ages of different parts of the human body, e.g. to find diseased organs or tissues. Measuring methylation levels in various biological samples is further reviewed in, e.g., U.S. patent application Ser. No. 15/025,185, which is incorporated herein by reference in its entirety, and other methods described herein.

In one aspect of the present invention, a method is provided for estimating methylation age using a whole blood biological sample. In another embodiment, the biological sample is individual blood cells, salvia, or a tissue sample. A biological sample can be obtained from a subject using techniques known in the art, e.g., removing blood directly from a subject's vein, or obtaining a dried blood spot sample. As used herein, a “dried blood spot sample” refers a biological sample comprising a blood sample blotted and dried on filter paper. “Dried blood spot samples” can be obtained by applying a few drops of blood (e.g., enough to saturate at least a portion of the filter paper) obtained by lancet from, e.g., finger, heal, or toe. The blood sample is allowed to thoroughly dry and is then stored at ambient room temperature. Samples can be analyzed by one skilled in the art. Dried blood spot samples are further reviewed in, e.g., U.S. Pat. No. 5,427,953, which is incorporated herein by reference in its entirety. Tissue samples can be obtained by one skilled in the art using, e.g., standard biopsy techniques for a given tissue.

In one embodiment, genomic DNA is extracted from the biological sample and used to measure methylation levels of the biological sample. As used herein, “genomic DNA” refers to chromosomal DNA. Genomic DNA can be extracted from a biological sample, e.g., whole blood, using commercially available kits, e.g., PureLink Genomic DNA Mini Kit, DNAzol BD Reagent, or MegaMAX-96 DNA Multi-Sample Kit (ThermoFisher Scientific; Waltham, Mass.). Reagents and kits useful for extracting genomic DNA from various biological samples (e.g., tissue samples, or salvia) are known in the art and can be determined by one skilled in the art.

In one embodiment, ribosomal DNA is extracted from the biological sample, e.g., whole blood. In one embodiment, the ribosomal DNA is extracted from the leukocytes of the whole blood. Reagents and kits useful for extracting ribosomal DNA from various biological samples (e.g., tissue samples, or salvia) are known in the art and can be determined by one skilled in the art.

### Risk Factors for Accelerated Aging

In one embodiment, a subject exhibit at least one risk factor of accelerated aging. In one embodiment of any aspect, the risk factor of accelerated aging includes, but is not limited to, use of tobacco products, use of alcohol, exposure to environmental toxins, a sedentary lifestyle, obesity, cancer, down syndrome, lack of nutritional intake, poor dietary habit, having complex diseases such as diabetes, CHD, hypertension, hyperlipidemia, and genetic risk predisposition. A risk factor can be, e.g., any behavior or symptom that can, or has been associated with decreasing the life span of a person.

In one embodiment, the methods described herein are used to determine if a subject at risk of accelerated aging exhibits accelerated aging. In one embodiment, a subject does not exhibit a risk factor of accelerated aging.

A skilled person, e.g., a skilled clinician, can determine if a subject exhibit at least one risk factor by standard methods, e.g., administering a self-evaluation, observing the subject, assessing a family and/or personal history of a subject, genetic testing (e.g., genome sequencing to identify a genetic mutation), or standard medical tests for diagnosing e.g., cancer, hypertension, or diabetes. Alternatively, a subject can determine if they exhibit at least one risk factor by self-evaluating their behavior and/or lifestyle. A subject that has determined that they exhibit at least one risk factor of accelerated aging can seek to obtain their methylation age, as measured using methods described herein, for example, to assess if they have accelerated aging.

### Pro-Health Therapy

In one embodiment, a subject who has been identified as having accelerated aging using the methylation clock described herein is administered a pro-health therapy, e.g., a therapeutic for the intended use of decreasing a subject's methylation age. In one embodiment, a subject who has been identified as having accelerated aging is administered at least two pro-health therapies.

In one embodiment, the pro-health therapy is any therapy that reduces a risk factors described herein, for example, losing weight, increased exercise, diet, reducing or stopping tobacco and/or alcohol use, or taking measures to reduce or increase metabolic measures, such as blood pressure, or cholesterol, or triglyceride levels.

In one embodiment, the pro-health therapy is caloric restriction. As used herein, “caloric restriction” refers to a reduction in a subject's total caloric intake in a 24 hour period. In one embodiment, a subject's caloric intake is reduced by at least 5%, 10%, 15%, 20%, 25%, 30%, 35%, 40%, 45%, 50%, 55%, 60%, 65%, 70%, 75%, 80%, 85%, 90%, or more, as compared to the subject's caloric intake prior to restriction.

A pro-health therapeutics can be a lifestyle change, e.g., reducing or completely removing risk factors for increased aging (e.g., losing weight, introducing an exercise regime, diet, reducing or stopping tobacco and/or alcohol use, or taking measures to reduce or increase metabolic measures, such as blood pressure, or cholesterol, or triglyceride levels). In one embodiment, a risk factor can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 85%, at least 90%, at least 95%, or more as compared to a reference level. As used herein, a reference level is the risk factor (e.g., the amount of caloric intake in a 24 hour period, or the number of cigarettes in a 24 hour period) present prior to being identified as having accelerated aging. As used herein, completely removing refers to the 100% removal of a risk factor.

A pro-health therapy can be increasing the amount of sleep a subject gets in a 24 hour period. In one embodiment, the sleep can be increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 85%, at least 90%, at least 95%, or more as compared to a reference level. As used herein, a reference level refers to the amount of sleep a subject gets in a 24 hour period prior to being identified as having accelerated aging.

A pro-health therapy can be a supplement, e.g., folate, or Vitamin B12, Vitamin B6, that affects the methylation state in a subject.

One skilled in the art will be able to determine an appropriate pro-health treatment for a subject who has been identified as having accelerated aging. The dosage or length of treatment will vary between pro-health treatments, and can be determined by one skilled in the art. The efficacy of the pro-health treatment in decreasing the methylation age of a subject can be determined by assessing a subject's methylation age during and/or after administration of a pro-heath treatment.

In one embodiment, the pro-health therapy results in demethylation of a methylation marker. In one embodiment, administration of a pro-health therapy decreases the level of methylation in a biological sample by at least 1%, by at least 2%, by at least 3%, by at least 4%, by at least 5%, by at least 6%, by at least 7%, by at least 8%, by at least 9%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 99%, or more as compared to an appropriate control. As used herein, the term “appropriate control” refers to the methylation level of a subject prior to the administration of a pro-health therapeutic.

In one embodiment, administration of a pro-health therapy decreases a subject's Δage such that it is equal to or less than zero. In one embodiment, administration of a pro-health therapy decreases a subject's rate of aging such that it is equal to or less than zero. In one embodiment, administration of a pro-health therapy decreases the methylation age of a subject by at least 1%, by at least 2%, by at least 3%, by at least 4%, by at least 5%, by at least 6%, by at least 7%, by at least 8%, by at least 9%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 99%, or more as compared to an appropriate control. As used herein, the term “appropriate control” refers to the methylation age of a subject prior to the administration of a pro-health therapeutic.

### Systems for Determining Methylation Age

A system for determining a methylation age related property of a subject, the system comprising: (a) an array; (b) an array reader configured to output methylation levels; (c) a display; (d) a memory containing machine readable medium comprising machine executable code having stored thereon instructions for performing a method; (e) a control system coupled to the memory comprising one or more processors, the control system configured to execute the machine executable code to cause the control system to: (i) receive, from the array reader, a methylation data set related to a methylation level of a blood sample of a subject; (ii) determine, based on the methylation data set, a methylation age related property using a regression model trained using subjects with an ethnicity that is the same as the subject's ethnicity; and (iii) output, to the display, the methylation age related property.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML ρage) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Kits

Described herein are kits for measuring the methylation age of a subject. In one embodiment, the kit consists of, consists essentially of, or comprises probes for detecting 100% of the methylation sites selected from the sites in Version 1 or Version 2 listed in Table 1. In one embodiment, the kit consists of, consists essentially of, or comprises probes for detecting at least 50% of the methylation sites selected from the sites in Version 1 or Version 2 listed in Table 1. In one embodiment, the kit consists of, consists essentially of, or comprises probes for detecting at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites in Version 1 or Version 2 listed in Table 1.

In one embodiment, the kit consists of, consists essentially of, or comprises probes for detecting all 38 methylation sites selected from the sites in Version 1 listed in Table 1. In one embodiment, the kit consists of, consists essentially of, or comprises probes for detecting at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 methylation sites selected from sites in Version 1 listed in Table 1.

In one embodiment, the kit consists of, consists essentially of, or comprises probes for detecting all 46 methylation sites selected from the sites in Version 2 listed in Table 1. In one embodiment, the kit consists of, consists essentially of, or comprises probes for detecting at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 methylation sites selected from sites in Version 2 listed in Table 1. In one embodiment, the probes for detecting the methylation sites in Model 1 or Model 2 in Table 2 are the primers listed in Table 4. In one embodiment, the kit consists of, consists essentially of, or comprises primers listed in Table 4.

In one embodiment, the kit consists of, consists essentially of, or comprises probes for detecting 100% of the methylation sites selected from the sites listed in Model 1 or Model 2 listed in Table 2. In one embodiment, the kit consists of, consists essentially of, or comprises probes for detecting at least 50% of the methylation sites selected from the sites listed in Table 2. In one embodiment, the kit consists of, consists essentially of, or comprises probes for detecting comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites in Model 1 or Model 2 listed in Table 2.

In one embodiment, the kit consists of, consists essentially of, or comprises probes for detecting all 10 methylation sites selected from the sites in Accessible Model 1 listed in Table 2. In one embodiment, the kit consists of, consists essentially of, or comprises probes for detecting at least 1, 2, 3, 4, 5, 6, 7, 8, or 9 methylation sites selected from sites in Accessible Model 1 listed in Table 2.

In one embodiment, the kit consists of, consists essentially of, or comprises probes for detecting all 15 methylation sites selected from the sites in Accessible Model 2 listed in Table 2. In one embodiment, the kit consists of, consists essentially of, or comprises probes for detecting at least 1, 2, 3, 4, 5, 6, 7, 8, 9, 10, 11, 12, 13, or 14 methylation sites selected from sites in Accessible Model 2 listed in Table 2.

In one embodiment, the kit consists of, consists essentially of, or comprises probes for detecting 100% of the methylation sites selected from the sites in Table 5. In one embodiment, the kit consists of, consists essentially of, or comprises probes for detecting at least 50% of the methylation sites selected from the sites in Table 5. In one embodiment, the kit consists of, consists essentially of, or comprises probes for detecting at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 5.

In one embodiment, the kit consists of, consists essentially of, or comprises probes for detecting all 80 methylation sites selected from Table 5. In one embodiment, the kit consists of, consists essentially of, or comprises probes for detecting at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 methylation sites selected from sites of Table 5.

In one embodiment, the kit consists of, consists essentially of, or comprises probes for detecting 100% of the methylation sites selected from the sites in Table 6. In one embodiment, the kit consists of, consists essentially of, or comprises probes for detecting at least 50% of the methylation sites selected from the sites in Table 6. In one embodiment, the kit consists of, consists essentially of, or comprises probes for detecting at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 6.

In one embodiment, the kit consists of, consists essentially of, or comprises probes for detecting all 67 methylation sites selected from Table 6. In one embodiment, the kit consists of, consists essentially of, or comprises probes for detecting at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67 methylation sites selected from sites of Table 6.

In one embodiment, the kit consists of, consists essentially of, or comprises probes for detecting 100% of the methylation sites selected from the sites in Table 7. In one embodiment, the kit consists of, consists essentially of, or comprises probes for detecting at least 50% of the methylation sites selected from the sites in Table 7. In one embodiment, the kit consists of, consists essentially of, or comprises probes for detecting at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 7.

In one embodiment, the kit consists of, consists essentially of, or comprises probes for detecting all 27 methylation sites selected from Table 7. In one embodiment, the kit consists of, consists essentially of, or comprises probes for detecting at least 1, 2, 3, 4, 5, 6, 7, 8, 9, 10, 11, 12, 13, 14, 15, 16, 17, 18, 19, 20, 21, 22, 23, 24, 25, 26 or 27 methylation sites selected from sites of Table 7.

In one embodiment, the kit consists of, consists essentially of, or comprises probes for detecting 100% of the methylation sites selected from the sites in Table 8.

In one embodiment, the kit consists of, consists essentially of, or comprises probes for detecting at least 50% of the methylation sites selected from the sites in Table 8. In one embodiment, the kit consists of, consists essentially of, or comprises probes for detecting at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the methylation sites selected from the sites listed in Table 8.

As used herein, the term “probes” as used herein are oligonucleotides capable of binding in a base-specific manner to a complementary strand of nucleic acid. In one embodiment, the probes consist of the sequences found herein in Table 4.

The term “probe” as used herein refers to a surface-immobilized molecule that can be recognized by a particular target as well as molecules that are not immobilized and are coupled to a detectable label. The terms “oligonucleotide” and “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res. 12:203 (1984), incorporated herein by reference.

The term “hybridization” as used herein refers to the process in which two single-stranded polynucleotides bind non-covalently to form a stable double-stranded polynucleotide; triple-stranded hybridization is also theoretically possible. Factors that can affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004, available at Affymetrix.com.

In one embodiment, the probes are mounted on a solid support. The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See, e.g., U.S. Pat. No. 5,744,305 for exemplary substrates, which is incorporated herein by reference in its entirety.

In one embodiment, the “probe” is a primer designed to amplify the gene containing the CpG position. The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

In one embodiment, the kit further comprising a device to collect a biological sample. Standard collection devices known for a given biological sample can be used. For example, collections devices for a blood sample can include, but are not limited, to a dried spot collection device, a finger prick collection device, or an arterial blood collection device. Collections devices for a saliva sample can include, but are not limited to, a collection tube for saliva or an oral swap. Collection devices for a tissue sample can include, but is not limited to, a biopsy collection device.

One skilled in the art is not necessarily required to obtain a biological sample. In one embodiment, the kit is for “at home use”, meaning it is intended that a subject will execute at least one step of the kit, for example, the subject obtains a biological sample using a collection device of the kit. In one embodiment, it is intended that the subject will execute all steps of the kit (e.g., obtain the sample, contact the probes with the obtained sample, and read the output (e.g., binding of the probes, or methylation age). Alternatively, the subject can execute at least one step of the kit, e.g., obtain the biological sample using a collection device of the kit and contact the biological sample with the probes, and then transport (e.g., mail) the kit to another facility where the output (e.g., binding of the probes, or methylation age) is read by a second individual. In one embodiment, the output is provided to the subject after the completion of the kit, e.g., via correspondence.

In another embodiment, the kit is intended for clinical purposes, and the steps are executed by one skilled in the art, e.g., a clinician.

The invention described herein can further be described in the following numbered paragraphs:
1) A method for determining a methylation age of a biological sample, the method comprising:
   a. measuring the methylation level of a set of methylation sites on ribosomal DNA (rDNA) of the biological sample; and
   b. determining the age of the biological sample using a statistical prediction algorithm based on the methylation level.
2) A method for determining a methylation age of a subject, the method comprising:
   a. collecting a biological sample from the subject;
   b. extracting genomic DNA for the collected biological sample;
   c. measuring a methylation level of a set of methylation sites on the ribosomal DNA; and
   d. determining the methylation age of the subject using a statistical prediction algorithm based on the methylation level.
3) A method for determining a Δage of a subject, the method comprising:
   a. collecting a biological sample from a subject;
   b. extracting genomic DNA for the collected biological sample;
   c. measuring a methylation level of a set of methylation sites on the ribosomal DNA;
   d. determining the methylation age of the subject using a statistical prediction algorithm based on the methylation level; and
   e. comparing the methylation age of the subject to a chronological age of the subject;
   wherein the Δage is the methylation age of the subject minus the chronological age of the subject.
4) The method of any of the proceeding paragraphs, wherein the biological sample is a blood sample or a tissue sample.
5) The method of any of the proceeding paragraphs, wherein the subject is male or female. 6) The method of any of the proceeding paragraphs, wherein the subject does not exhibit a risk factor of accelerated aging.
7) The method of any of the proceeding paragraphs, wherein the subject exhibits at least one risk factor of accelerated aging.
8) The method of any of the proceeding paragraphs, wherein the risk factor of accelerated aging is selected from the group consisting of: use of tobacco products, use of alcohol, exposure to environmental toxins, sedentary lifestyle, obesity, cancer, down syndrome, lack of nutritional intake, poor dietary habit, having complex diseases such as diabetes, CHD, hypertension, hyperlipidemia, and genetic risk predisposition.
9) The method of any of the proceeding paragraphs, wherein the set of methylation sites are the methylation sites in Table 1, Table 2, Table 5, Table 6, Table 7, or Table 8.
10) The method of any of the proceeding paragraphs, wherein the set of methylation sites comprise at least 90%, at least 80%, at least 70%, at least 60%, at least 50% of the sites of Table 1, Table 2, Table 5, Table 6, Table 7, or Table 8.
11) The method of any of the proceeding paragraphs, wherein the set of methylation sites comprise each of the sites of Table 1, Table 2, Table 5, Table 6, Table 7, or Table 8.
12) The method of any of the proceeding paragraphs, wherein the statistical prediction algorithm comprises:
   a. identifying at least two coefficients found in Table 1, Table 2, Table 5, Table 6, Table 7, or Table 8 in a biological sample;
   b. multiplying each of the at least two coefficients with its corresponding CpG's methylation level to output a value for each of the at least two coefficients;
   c. find a sum of values of (b) for each identified coefficient;
   d. adding a recalibration intercept to the summed values of (c);
   e. calculating the natural exponentiation of (d), wherein the exponentiation is the predicted methylation age of the subject.
13) The method of any of the proceeding paragraphs, wherein a Δage greater than zero is an indicator of accelerated aging of the individual.
14) The method of any of the proceeding paragraphs, further comprising administering a pro-health therapy to a subject with a Δage greater than zero.
15) The method of any of the proceeding paragraphs, wherein the pro-health therapy is a therapy that decreases the methylation age of the subject.
16) A method for determining a methylation age of a cell, the method comprising:
   a. extracting genomic DNA from the cell or population thereof;
   b. measuring a methylation level of a set of methylation sites found in Table 1, Table 2, Table 5, Table 6, Table 7, or Table 8 on the ribosomal DNA; and
   c. determining the methylation age of the cell based on the methylation level.
17) The method of paragraph 16, wherein the cell is a mammalian cell.
18) The method of any of the proceeding paragraphs, wherein the cell is a pluripotent cell.
19) The method of any of the proceeding paragraphs, wherein the cell is a stem cell.
20) The method of any of the proceeding paragraphs, wherein the cell is an induced pluripotent stem cell.
21) A kit comprising probes for detecting methylation sites found in Table 1, Table 2, Table 5, Table 6, Table 7, or Table 8.
22) The kit of paragraph 21, wherein the set of probes comprise at least 90%, at least 80%, at least 70%, at least 60%, at least 50% of the sites of Table 1, Table 2, Table 5, Table 6, Table 7, or Table 8.
23) A system for determining a methylation age related property of a subject, the system comprising:
   an array;
   an array reader configured to output methylation levels;
   a display;
      a memory containing machine readable medium comprising machine executable code having stored thereon instructions for performing a method;
      a control system coupled to the memory comprising one or more processors, the control system configured to execute the machine executable code to cause the control system to:
      receive, from the array reader, a methylation data set related to a methylation level of a blood sample of a subject;
      determine, based on the methylation data set, a methylation age related property using a regression model trained using subjects with an ethnicity that is the same as the subject's ethnicity; and
      output, to the display, the methylation age related property.
24) The system of paragraph 23, wherein the methylation level of a blood sample of the subject is the method level of leukocytes of the subject.
25) The method of any of the proceeding paragraphs, wherein the blood is whole blood, peripheral blood, or cord blood.
26) The method of any of the proceeding paragraphs, wherein the tissue sample is selected from the group consisting of: skin tissue, breast tissue, ovarian tissue, liver tissue, kidney tissue, lung tissue, pancreatic tissue, thyroid tissue, thymus tissue, spleen tissue, bone marrow, lymphoid tissue, epithelial tissue, endothelial tissue, ectoderm tissue, nervous tissue, connective tissue, and mesoderm tissue.
27) A method of reducing a methylation age in a subject, the method comprising:
   a. receiving the results of an assay that diagnoses a subject of having advanced methylation aging; and
   b. administering at least one pro-health therapy, wherein the pro-health therapy reduces the methylation age of the subject as compared to an appropriate control.
28) The method of paragraph 27, wherein the appropriate control in the methylation age of the subject prior to administration.
   therapy reduces the methylation age of the subject as compared to an appropriate control.

### Examples

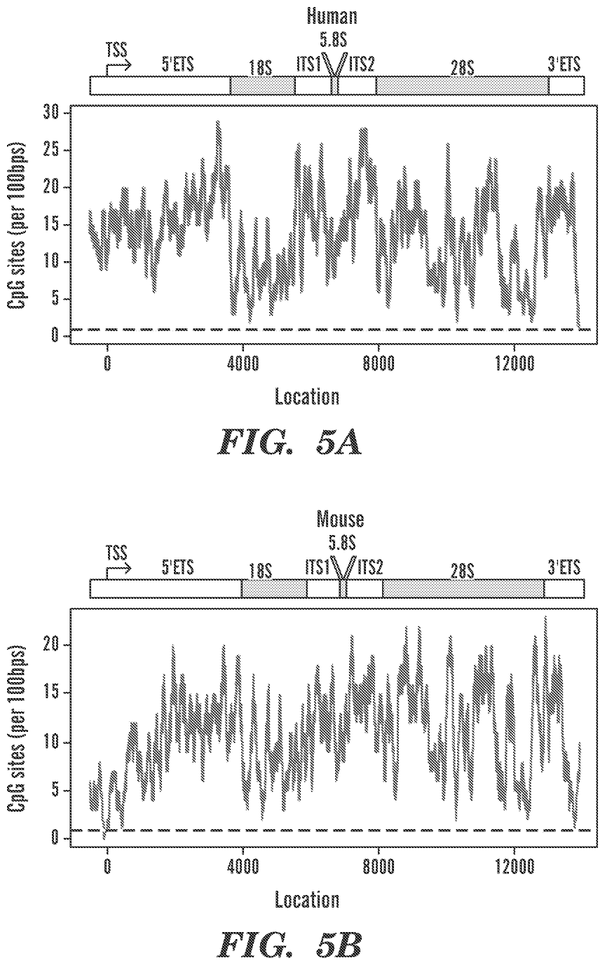

Aging is a universal trait that is accompanied by dramatic changes in myriad biological attributes across molecular, cellular, and organismal levels (1). However, the development of mechanistic markers of organismal or molecular aging has remained a challenge. Telomere attrition, for instance, impacts cellular longevity through an undisputable mechanism, but the efficacy of telomere length as an aging biomarker appears equivocal (2). Notably, groups of CpGs scattered along the genome have been used to indicate age (3); however, they were statistically identified from thousands of CpGs and are neither functionally related nor evolutionarily conserved (4-7). The evolutionary conserved ribosomal DNA (rDNA) gives origin to the nucleolus, an energy intensive nuclear organelle that is the site of transcription of over 70% of all cellular RNAs (the ribosomal rRNAs). As a major hub influencing myriad processes, the rDNA/nucleolus has been directly implicated in aging and longevity from yeast to humans (8-12). Interestingly, the rDNA is a main target of the DNA methylation machinery that silences supernumerary rDNA units and regulates nucleolar activity (13, 14). Each unit of the tens to hundreds of 45S rDNA repeats harbors over 1500 CpGs, or more than 10 CpGs per 100 nucleotides (FIG. 5). The segment, however, remains missing from genome assemblies and is typically neglected in most genomic studies.

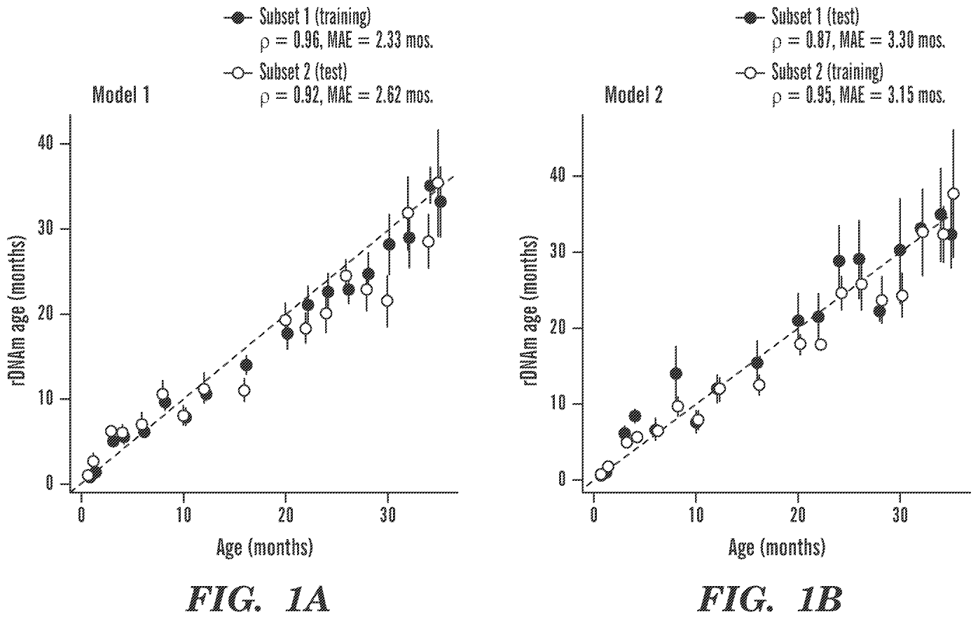

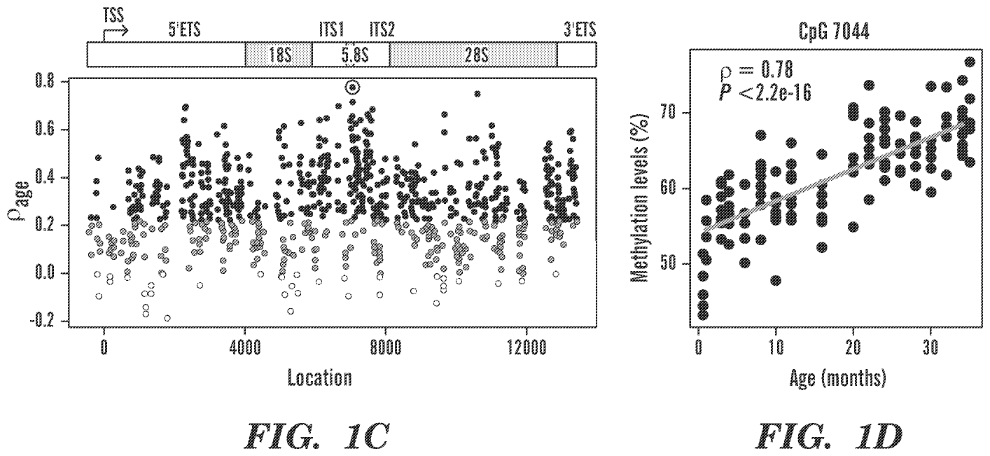

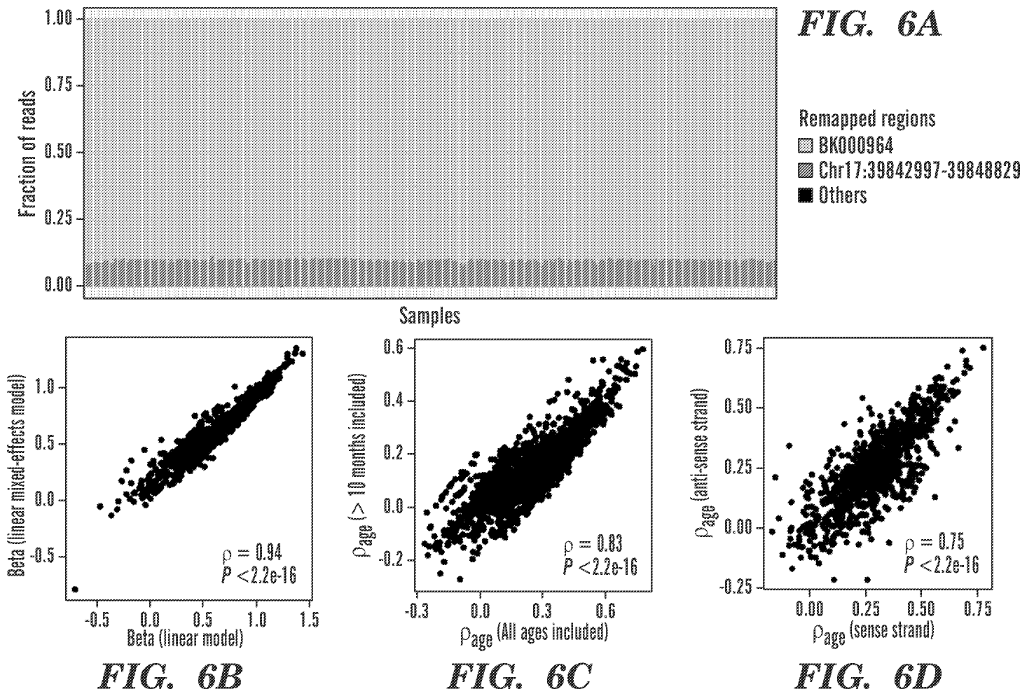

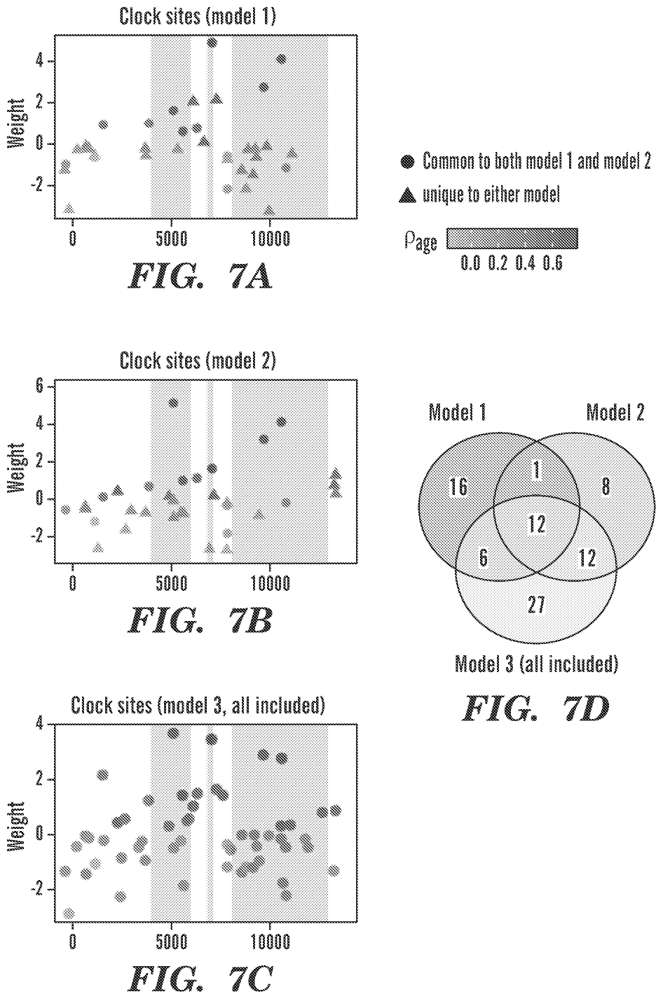

Whether CpG methylation in the rDNA array is sufficient to predict chronological age is determined herein. To address the issue, a recently published dataset with whole-blood reduced representative bisulfate sequencing (RRBS) from C57BL/6 mice at ages ranging from 0.67 to 35 months (6) (16 age stages, sample information, data not shown) was examined. It was determined that over 99% of rDNA reads are accurately mapped (FIG. 6a), that batch effects are uninfluential (FIGS. 6b and 6c; see herein Methods and materials below), and identified 816 CpGs that are informative (depth 50) in all samples. The dataset into was then divided two subsets with equal numbers of individuals in each age, and applied an elastic-net regression model to each set. The procedure yielded two models using each subset for training and the other subset for performance testing. For each of the training sets a group of CpGs (or clock sites) and site-specific weights were identified, and calculated a predicted age (or rDNAm age) (see herein Methods and materials below). In both models, the clock was able to achieve strong fit in the training subsets (FIGS. 1a and 1b, Spearman's ρ=0.96 and 0.95 between rDNAm age and chronological age for model 1 and 2, respectively; P<2.2e-16). Applying the clock sites to the test sets, slightly lower but still remarkably strong correlations were observed between rDNAm age and chronological age (FIGS. 1a and 1b, Spearman's ρ=0.92 and 0.87, P<2.2e-16). These correlations are only slightly lower than those from a model built on genome-wide CpGs(6). The median absolute errors (MAEs) in the test sets were 2.62 and 3.30 months (FIGS. 1a and 1b). Specifically, model 1 and 2 yielded 35 and 33 clock sites (FIGS. 7a and 7b), with 13 sites shared by both models and yielding a similar coefficient in each model. Thus, in order to maximize statistical power, all samples from the two subsets were merged and a similar training process was employed to build a unified model. As a result, this model yielded 57 clock sites, with 30 sites shared to at least one of the above two models (FIGS. 7c and 7d). The estimated MAE through cross-validation is 2.51 months. The discrepancy in the sets of clock sites may reflect vagaries in elastic-net selection among a group of correlated CpGs that index the same underlying process(15). Interestingly, clock sites are located across the rDNA gene region and include CpGs with weak age-associations (FIG. 7). These results indicate that rDNA methylation is sufficient to estimate chronological age in mice.

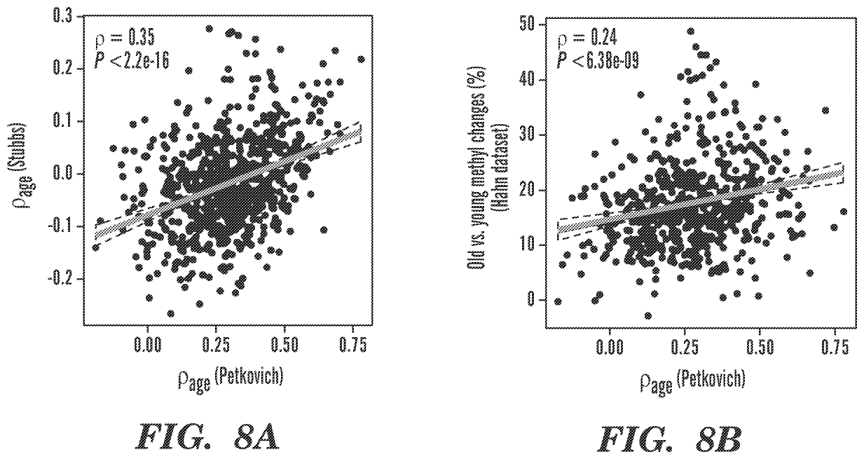

The reasonable performance of rDNAm clock sites raises the question of how methylation of individual CpG site changes during aging. To explore this, each of 928 CpGs (depth 50 in over 90% samples) were correlated with age. Strikingly, 620 sites (66.8%) were observed to be located almost uniformly along the transcribed and promoter regions of the rDNA displayed statistically significant positive correlation with age (ρage>0; FDR<0.01; FIG. 1c). The site with the strongest association is CpG 7044 (FIG. 1d, ρage=0.78, P<2.2e-16), located 10 nucleotides downstream of the 5.8S coding sequence. The hypermethylation is consistent for both strands of the rDNA sequence (FIG. 6d) and in agreement with observations of increased methylation at a few sporadic CpG sites on rDNA (16). To further validate the age association of rDNA methylation in mice, two additional datasets were analyzed (7, 17). Both data confirmed that CpGs with higher ρage are also more likely to be hypermethylated in elder mice, despite differences in tissue type and strains. (FIG. 8, Spearman's ρ≥0.24, P≤6.38e-9; and data not shown). Finally, application of the rDNA methylation clock to these alternative strains/tissues yielded high correlation between relative rDNA age and chronological age (r=0.80). Taken together, these results demonstrated the age-associated hypermethylation of CpGs along the rDNA sequence in mice and highlight the usefulness of the rDNA methylation clock.

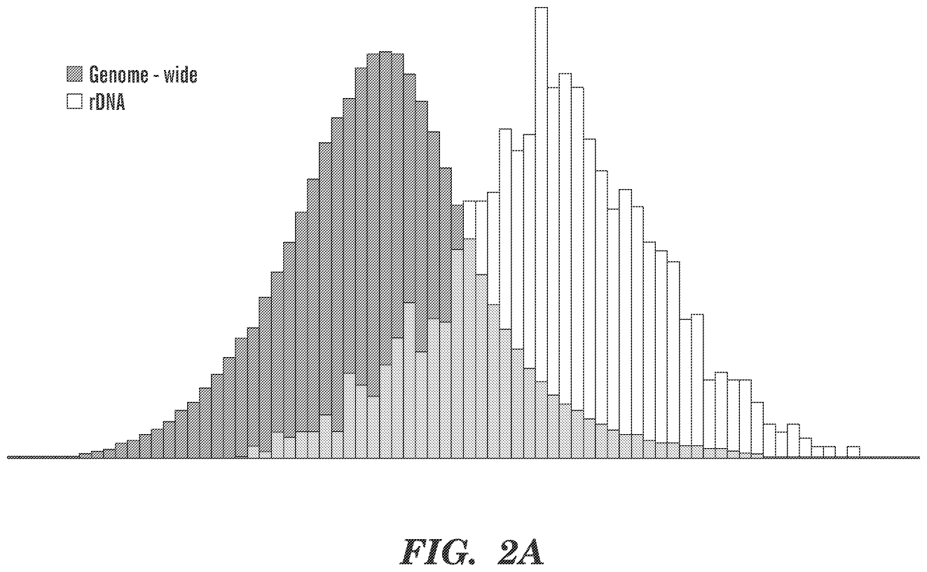

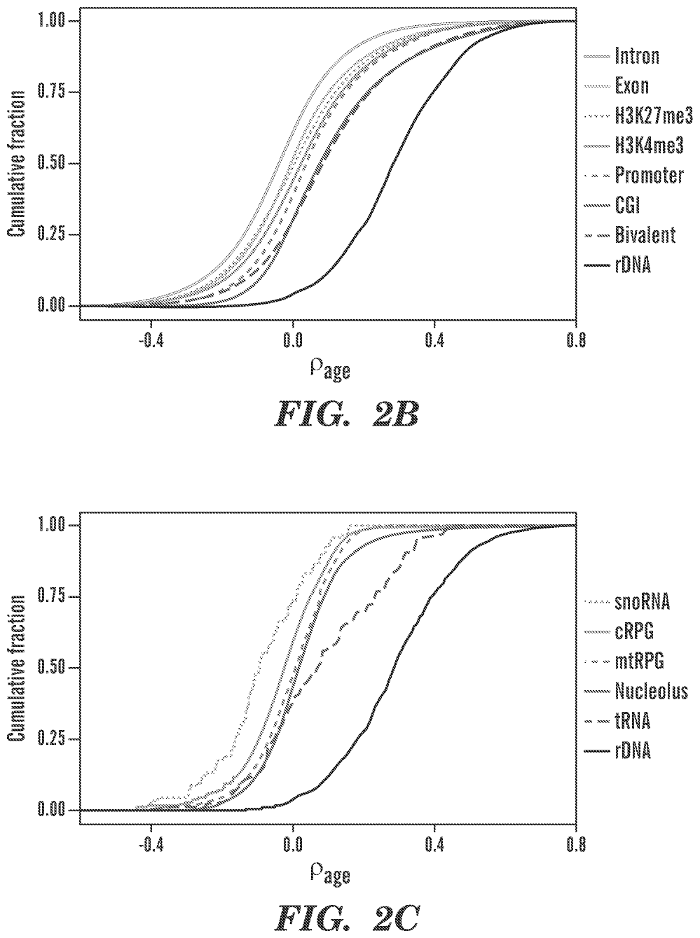

The strong age-associated hypermethylation of the rDNA prompted the interrogation of other genomic regions and functional classes. First, DNA methylation changes across the entire genome were examined. It was found that most sites showed little to no correlation with age, with a small bias towards loss of DNA methylation with age. The proportion of CpGs with positive correlation (ρ>0.2) is markedly lower than that of rDNA (8.03% genome-wide vs. 71.8% in rDNA; FIG. 2a). A closer examination into different classes of genomic segments showed a similar distribution of age-association for CpG located within intron, exons, and promoters, with CpG islands (CGIs) and domains of bivalent chromatin (marked by both H3K27me3 and H3K4me3) displaying a slight shift towards hypermethylation (FIG. 2b), which coincides with previous observations (17, 18). However, the methylation of the rDNA stood out with a significant bias towards positive associations with age. Next, the promoters of functionally coherent classes related to rDNA/nucleolus function were examined. It was found that genes encoding protein components of the cytoplasmic and mitochondrial ribosome (cRPGs and mRPGs) did not show biases towards higher or lower promoter methylation with age (FIG. 2c). Similarly, as a class, genes encoding proteins that localize to the nucleolus or encoding small nucleolar RNAs (snoRNAs) did not show higher or lower correlations with age than expected from genome-wide patterns. Interestingly, the Pol III transcribed tRNAs displayed a pattern that most closely resembled that in the rDNA, with a bias towards positive associations with age (FIG. 2c). tRNA methylation is used as a mechanism to suppress Pol III transcription (19) and might lead to decreased tRNA transcription through aging. Collectively, these observations indicate that age-associated hypermethylation is neither universally manifested across the genome nor ubiquitously adopted by Pol II transcribed genes that are functionally related to ribosomal biogenesis and the nucleolus. The observations highlight the tRNAs and the rDNA as hotspots for age-association.

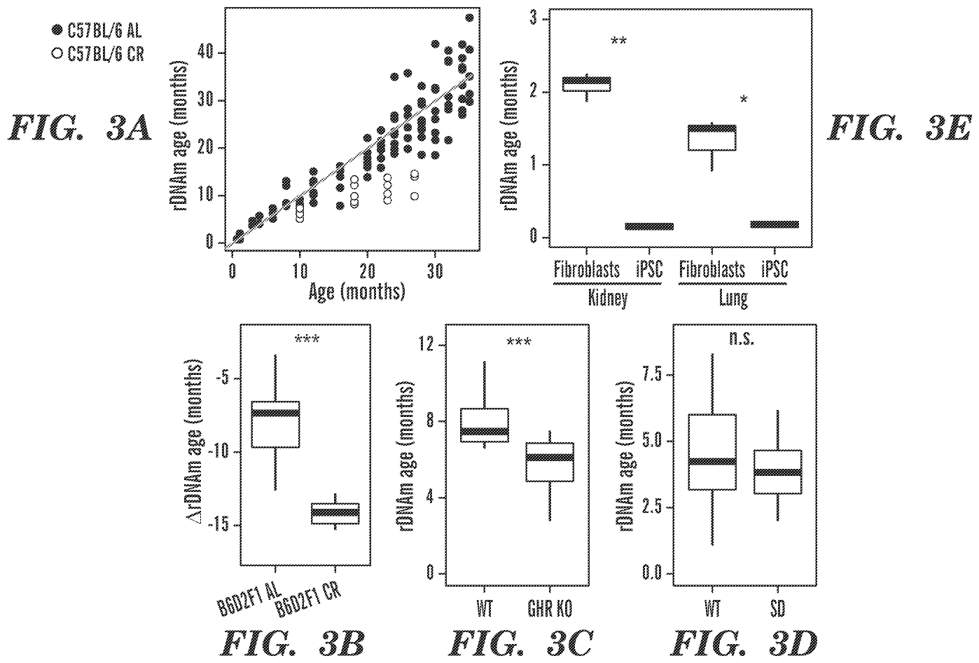

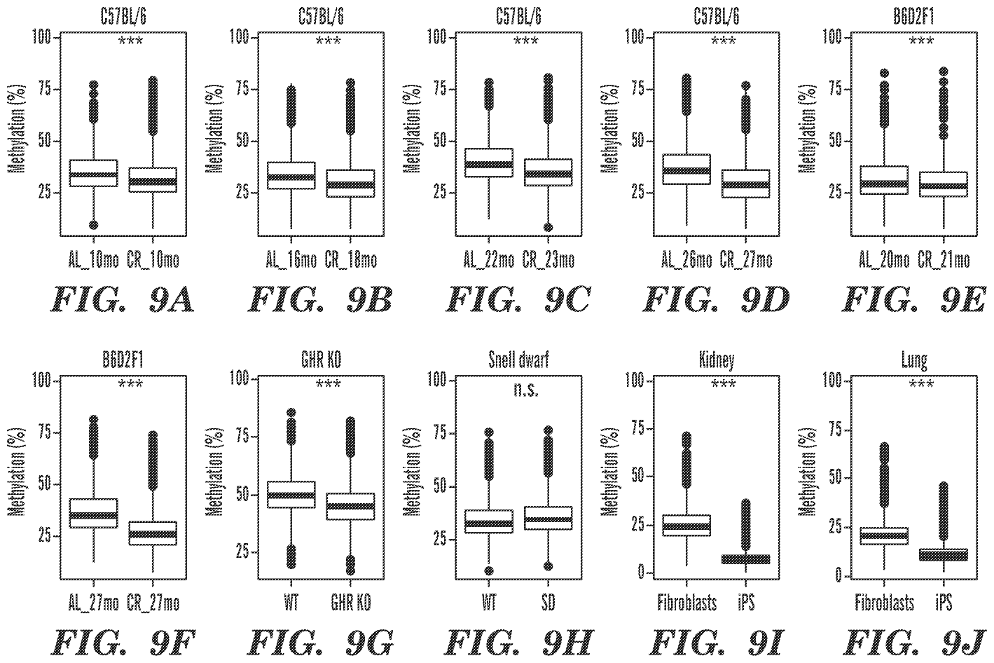

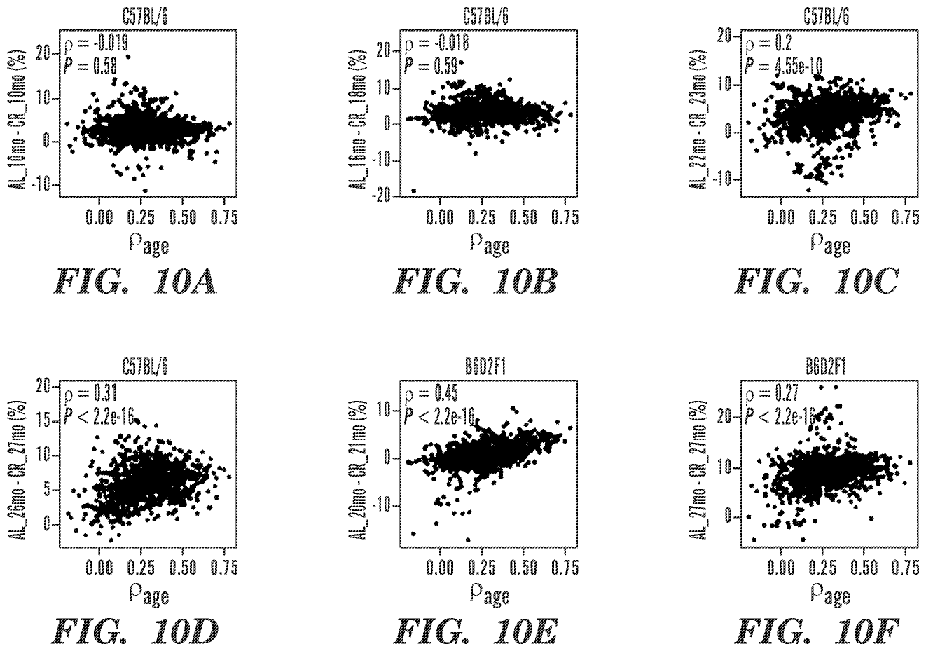

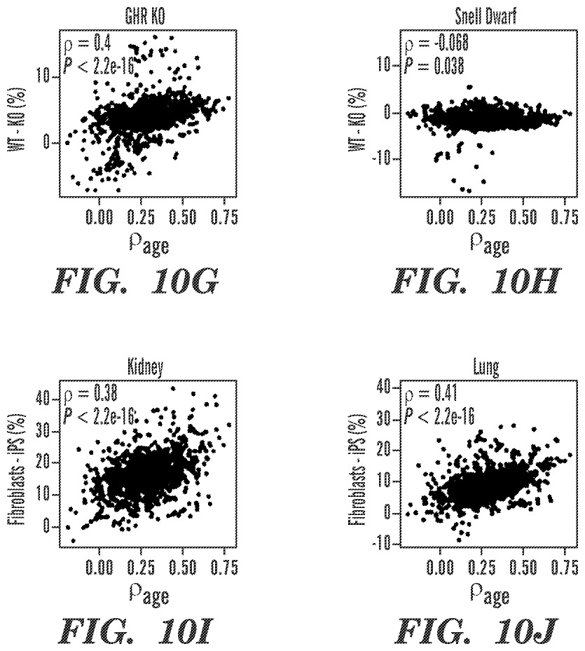

It was next examined whether the rDNAm clock is responsive to genetic and environmental interventions that are known to modulate lifespan. Calorie restriction (CR) has long been reported to extend lifespan and retard aging. For the C57BL/6 mice subjected to CR starting at 14 weeks old, an overall lower rDNAm age was observed compared to their ad libitum (AL) controls (FIG. 3a, one-tailed t-test of the differences between rDNAm age and chronological age, P=1.08e-8), with the exception of the youngest CR mice that were 10 months old (FIG. 3a, P=0.068). The CR effect remained obvious when instead examining the B6D2F1 strain mice (FIG. 3b, P=3.56e-6). The rDNAm clock to two genetic slow-aging mice models yielded distinct outcomes: the slow aging full-body growth hormone receptor knockout (GHR KO) mice showed significant reduction in rDNAm age (FIG. 3c, P=0.00052) compared to wild-type controls, whereas no significant reduction was observed in snell dwarf (SD) mice (FIG. 3d, P=0.30). Moreover, lower rDNAm ages for induced pluripotent stem cell (iPSC) lines was also consistently observed significantly relative to their kidney and lung fibroblasts progenitors (FIG. 3e, P<0.015). The change of methylation for the intervention groups was further examined. As expected, except for SD mice, both CR and GHR KO mice showed significant decrease in rDNA methylation (FIG. 9A-9I), especially for CpG sites with strong age-associations (FIG. 10A-10J), compared to their respective controls. Such pattern also holds for iPSC lines compared to their relative fibroblasts (FIGS. 9A-I and 10A-10I). Overall, these results indicate that genetic and environmental interventions known to influence longevity can impact rDNA methylation and modulate the rDNAm clock in a coherent manner.

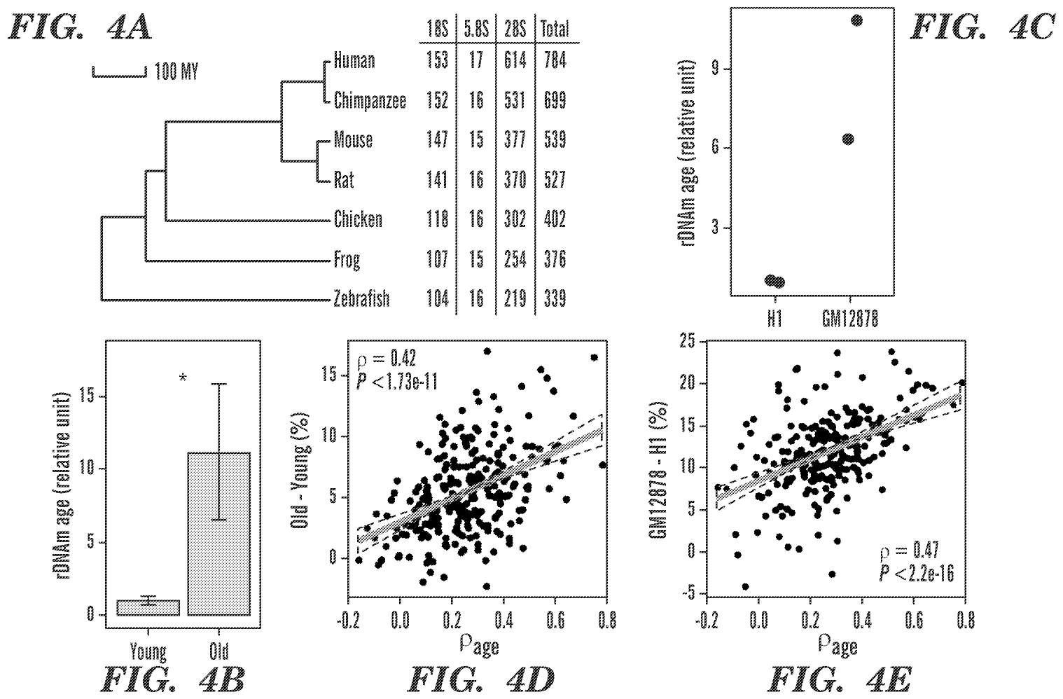

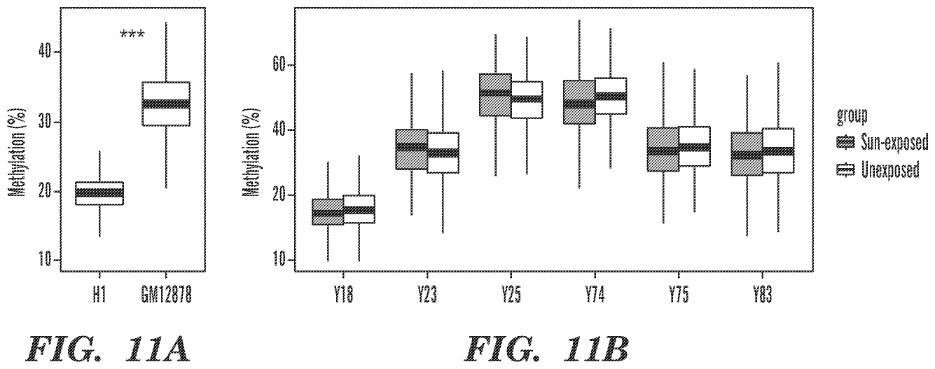

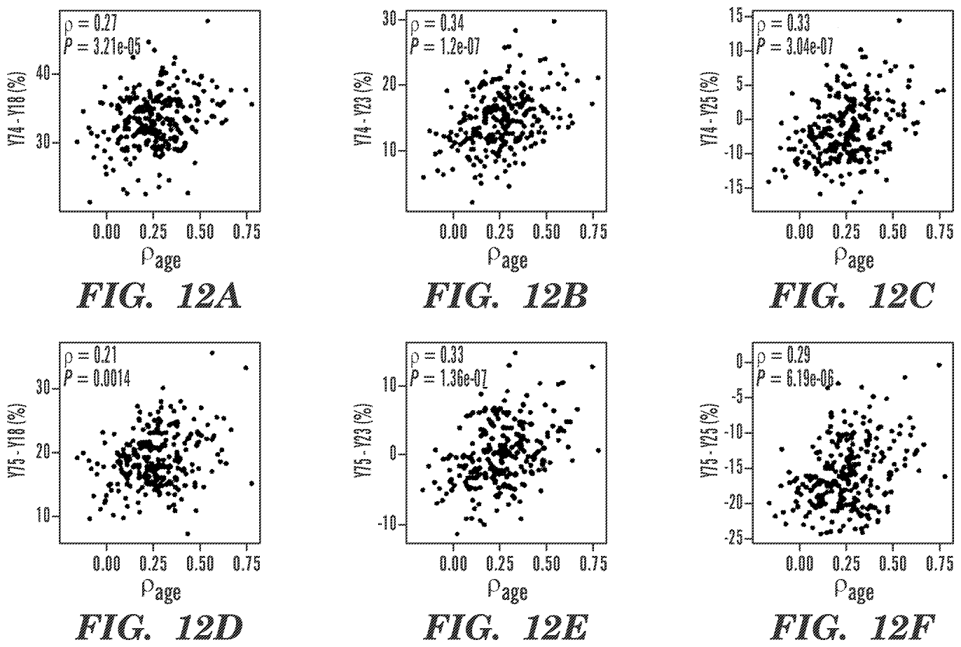

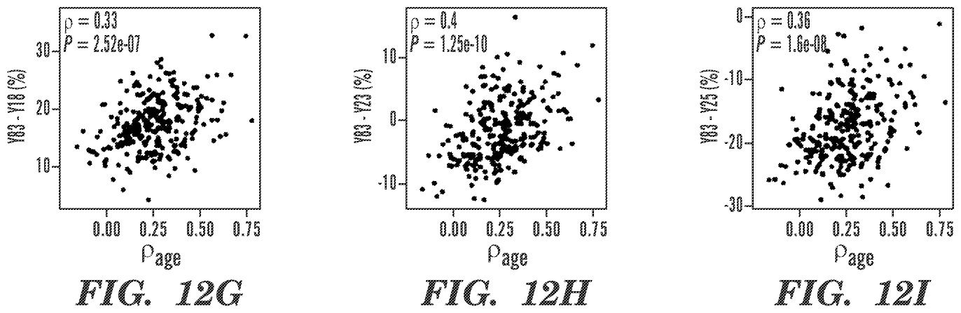

As the most evolutionary conserved segment of the genome, the rDNA is essential for both prokaryotic and eukaryotic life and has been the marker of choice for phylogenetic analyses of ancient speciation events. Indeed, a large proportion of CpGs from rRNA coding regions are conserved across vertebrates, with over 40% (338/784) of human CpGs detected with stringent cutoff (see Methods) in species as divergent as zebrafish (FIG. 4a). Therefore it was asked whether the rDNA methylation clock trained in mice is evolutionarily conserved and able to gauge age in other species. To address this, two human datasets were considered: one includes skin samples of old (>70 years old) and young (<30 years old) individuals (20); the other one comprises the B-lymphocyte cell GM12878 from a healthy adult and the embryo stem cell H1. An age model was then built with the mouse cohort exclusively using mouse-human homologous CpGs. The age model yielded good estimates in mice that are comparable to those obtained using the full set of sites (MAE=3.15 months). Notably, applying the model to humans, higher relative rDNAm could also be observed age in elder skin samples compared to younger ones (FIG. 4b, one-tailed t-test, P=0.04), and in GM12878 compared to H1 (FIG. 4c). Further analyses of the cell lines showed lower methylation levels for the whole rDNA regions in H1 (FIG. 11a). For the skin samples, interestingly, considerable inter-individual variability was observed, while for each individual the methylation between sun-exposed and unexposed samples were almost identical (FIG. 11b). Despite this, the model still separated the old from young individuals (FIG. 4b). To understand this, the homologous CpGs were examined, and strong positive correlation was observed between their ρage in mice and the increase in methylation in old relative to young skins, or in GM12878 relative to H1 (FIGS. 4d and 4e, Spearman's ρ≥0.42, P≤1.73e-11). Importantly, such correlation still held when instead considering every old vs. young comparison for the skin samples (FIG. 12A-I), suggesting consistent aging effect despite the existence of inter-individual variability. Collectively, these data showed that human rDNA also undergoes hypermethylation during aging and revealed an evolutionarily conserved rDNA methylation clock. The clock is also in agreement with the massive over-representation of ribosomal protein genes among genes whose expression is associated with age in both humans and mice studies (21, 22).

Data presented herein reveal an evolutionarily conserved rDNA methylation clock, which emerges from a strong positive association between rDNA methylation and age. Recent studies reported that the array exerts manifold functional consequences on genome integrity, cellular metabolism and heterochromatin maintenance (16, 23-25), with well documented downstream impacts on aging at both cellular and organismal levels. The array is also key landmark around which the rest of the genome is organized in the nucleus (26, 27) and is associated to gene expression variation across the genome (28). Euchromatic gene expression and silencing is influenced by proximity to the rDNA/nucleolus (29). One model through which the rDNA exerts epigenetic control on the genome is by altering the availability of limited chromatin regulators (30). Overall, variation in rDNA methylation likely reflects conserved nucleolar properties that not only respond to cellular regulation but also influence biological processes that ultimately impact aging. Thus, the ribosomal clock is not only mechanistically sound but also readily deployable to aging and population studies in natural settings and wild-organisms across the spectrum of eukaryotes.

Methods and Materials

Description of sequencing data—Five whole-genome and reduced representative bisulfite sequencing datasets (WGBS and RRBS) were used in this study. Below is a brief description of these datasets, and data not shown.

The Petkovich dataset(6) include 255 samples: 1) 153 C57BL/6 strain mice with 18 age stages ranging from 0.67 to 35 months, and 10 B6D2F1 strain mice with 2 age stages; 2) 20 C57BL/6 and 12 B6D2F1 mice subjected to calorie restriction; 3) two slow-aging models, 15 whole-body growth-hormone-receptor knockout (GHRKO) and 10 snell dwarf, and their corresponding wild-types (11 and 12 samples); 4) 6 fibroblasts of lung and kidney (3 from each) from 10-week-old mice, and the 6 iPSC lines derived from them. Except for fibroblasts and the corresponding iPSC lines, whole blood was used for RRBS in all the other samples.

The Stubbs dataset(7) includes RRBS of 4 mice tissues (cortex, heart, liver and lung) at 4 age stages (1, 14, 27 and 41 weeks). All 62 samples are male C57BL/6-BABR strain mice.

The Hahn dataset(17) includes liver WGBS of mice at two ages (5 months and 26 months, 3 samples in each group). All samples are female C3B6F1 strain mice.

The Vandiver dataset(20) includes skin WGBS of 3 old (>70 years old) and 3 young (<30 years old) humans. All individuals had epidermis of both sun-exposed and unexposed skin sequenced.

The WGBS of human embryo stem cells (H1), and B-lymphocyte cells (GM12878) were downloaded from the ENCODE portal (https://www.encodeproject.org)(31). GM12878 was derived from a mother with unknown age and then immortalized.

Obtaining rDNA sequences—The consensus 45S rDNA sequences of human, mouse, rat, chicken and frog were from GenBank (accessions: U13369.1, BK000964.3, NR 046239.1, KT445934.2 and X02995.1, respectively). To obtain the rDNA sequences of chimpanzee and zebrafish, Blat(32) (e.g., found on the world wide web at https://genome.ucsc.edu/cgi-bin/hgBlat) was used to map human rDNA against their genome assemblies (panTro5 and danRer11). Specifically, the chrUn_NW_015976995 v1 contig (18S: 7807-9675, 5.8S: 6583-6739 and 28S: 283-5419; minus strand) in chimpanzee and chr5: 820041-826807 (18S: 824921-826807, 5.8S: 824487-824644 and 28S: 820041-824135; minus strand) were found with the highest similarities and selected. The downloaded sequences of human and mouse were further modified to contain the promoter (defined as the last 500 bps of the units) and the transcribed regions (including 5′ ETS, 18S, ITS1, 5.8S, ITS2, 28S and 3′ ETS) for mapping purpose.

Data processing—After evaluating the sequencing quality using FastQC (e.g., found on the world wide web at https://www.bioinformatics.babraham.ac.uk/projects/fastqc/), Trim_Galore! Was used (e.g., found on the world wide web at https://www.bioinformatics.babraham.ac.uk/projects/trim_galore/) to trim the 3′ adaptors as well as low quality bases (BAQ<20). The ‘--rrbs’ option was additionally used for RRBS reads to remove the filled-in bases. Bismark(33), was then used, which invoked bowtie2 v2.3.1(34) to map the bisulfite sequencing reads onto the modified rDNA reference sequences of respective species. The methylated and unmethylated reads were counted using the ‘bismark_methylation_extractor’ script.

To examine whether reads derived from other genomic regions were incorrectly mapped onto the rDNA reference, the rDNA mapped reads from the Petkovich dataset were realigned onto mice genome, with the modified BK000964.3 sequence included. It was observed that over 99% of the reads can be specifically realigned onto BK000964.3 as well as a homologous segment on chromosome 17 (FIG. 6a), supporting that almost all of the rDNA mapped reads are indeed from the rDNA sequence.

Examination of batch effects—Petkovich et al(6) have explored batch factors in detail, and suggested no perceptible effects on the methylation of genome-wide CpGs. Here it is further examined whether batch effects can be observed for rDNA CpGs. This dataset includes three confounding variables: adaptor numbers, library numbers and flow cells. Since library numbers are almost linearly correlated with flow cells, adaptor numbers and library numbers were instead only considered. The linear mixed-effects model method from Petkovich et al(6) was first adopted. That is, in a linear mixed-effects model, age and methylation level (of each CpG site) are the response and fixed independent variable, while the confounding factors are random effects. The coefficient of each CpG is then compared with that from a simple linear model, where age and methylation are the response and independent variable. Indeed, the two coefficients are highly correlated (FIG. 6b, Spearman's ρ=0.94, P<2.2e-16). Moreover, as has been suggested (6) that there's certain redundant anti-correlation between library number (flow cell) with age, it was also observed that younger mice tend to have larger library numbers, but those elder than 10 months actually have a rather random distribution (ρ=−0.07, P=0.5). Mice elder than 10 months were only used to estimate the correlation of each CpG with age, and the newly calculated coefficients were compared with those calculated using mice from all age stages. As a result, a very strong correlation was observed between these two coefficients (FIG. 6c, ρ=0.83, P<2.2e-16), only with the newly calculated ones having smaller values (possibly due to smaller sample size). Together, these analyses suggested inconsequential, if any, batch effects on the results.

Building the methylation age clock—The original Petkovich dataset has already grouped 141 control-fed C57BL/6 strain mice into two subsets (12 mice 0.67 and 1.17 months old were not included). However, the samples in the two subsets are very unbalanced among age stages. Since the set of rDNA CpGs are much smaller, such unbalance may lead to bias toward certain CpGs in one subset but not the other one. Therefore, 153 control-fed C57BL/6 mice (the 12 younger mice also included) were re-assigned the into two subsets randomly, with the nearest numbers in each age group for the two subsets.

To build the methylation age clock, the elastic-net regression model implemented in the glmnet library(35) in R was used. This model applies multivariate linear regression with the predict and response variables being the methylation levels of CpGs and the logarithm transformed age, respectively. In addition, the model exerts extra constraint on the coefficients of predict variables by adding penalty to the coefficients using the combination of lasso and ridge regulation methods. Specifically, for the set of n mice and p CpG sites, the model finds the set of coefficients, β, that can minimize the following term:

$$\frac{1}{2 n} {\sum }_{i = 1}^{n} {( {y}_{i} - {β}_{0} - {\sum }_{j = 1}^{p} {β}_{j} {x}_{i j} )}^{2} + λ [ \frac{1 - α}{2} {\sum }_{j = 1}^{p} {β}_{j}^{2} + α {\sum }_{j = 1}^{p} \vert {β}_{j} \vert ]$$

Here x_ij is the methylation level of ith mouse at jth CpG, and y_i is the log transformed age of ith mouse. Moreover, λ>0 is a tuning parameter that regulates the overall penalty against the coefficients, and 0<α<1 represents a compromise between ridge (α=0) and lasso (α=1). In the modeling process, a was set to 0.5(5, 6), while λ was chosen through ten-fold cross-validation following the one-standard-error rule, e.g., the value one standard error larger than the one that minimizes the mean cross-validated error.

The feature selection nature of the method makes it possible to pick a subset of CpGs to build the model (the rest have coefficients of 0). However, repeating the training process using even the same samples is likely to yield different combinations of CpGs, since the number of input CpGs are much larger than the sample size. To account for such stochasticity, we iterated the division-training-testing procedure for 10,000 times to see how well the method works on average. The models applied inter-specifically were built by using homologous CpGs that have enough reads mapped (>=6 for genomic CpGs and >=50 for rDNA CpGs) in all samples of both species. The trainings and tests were processed similarly.

Noticing the vast differences in lifespan and developmental pace for distinct species, we first calculate relative age for applications in which the model is trained in one species and then translated to another. Relative ages are then transformed to chronological age based on the maximum lifespan interval of each species.

The unified models based on all 153 control-fed mice were built separately by applying either the 816 rDNA CpGs or only the homologous CpGs. To evaluate the accuracy of the models, a leave-one-out cross-validation method was used. Specifically, each time 152 out of the 153 samples were selected to build an elastic-net model, while the remaining one was used to calculate an absolute error by fitting the model. After repeating this procedure exhaustively, i.e., 153 times, all samples were left out exactly once, and 153 absolute error values were yielded. It was then considered the median value of these absolute errors as the MAE of the respective unified model. The models using all but one samples were expected to be almost identical to the unified models, therefore the estimated MAEs were likely to be only negligibly biased.

Defining functional classes and genome regions—Mouse genes from Ensembl(36) release 90 were used to identify exon, intron and promoter regions, with pseudogenes excluded. For each gene, the region from 1000 bps upstream to 500 bps downstream the transcription start site was considered its promoter. The location of CpG islands were downloaded from UCSC table browser(37). The processed chromatin peaks of H3K27me3 and H3K4me3 modifications of megakaryocyte cells were from the GEO database (accession numbers: GSM946523 and GSM946527), and were converted from mm9 to mm10 using the UCSC liftover tool (https://genome.ucsc.edu/cgi-bin/hgLiftOver). The overlaps of the two kinds of peaks were considered bivalent chromatin. The group of snoRNA genes were from Ensembl annotation. Cytoplasmic ribosomal protein genes (cRPGs, genes under GO terms GO:0022625 and GO:0022627), mitochondrial ribosomal proteins (mtRPGs, GO:0005762 and GO:0005763) and nucleolar genes (GO:0005730) were downloaded from Ensembl biomart. The tRNA genes were from GtRNAdb(38). For snoRNA and tRNA genes, the regions from 100 bps upstream the transcription start sites to 3′ end sites were considered.

Identifying homologous rDNA CpG sites between species—Given to the lack of similarity in ETS and ITS across species, only the 3 coding regions were considered. For each region, ClustalW(39) (e.g., found on the world wide web at http://www.genome.jp/tools-bin/clustalw) was used to align the sequences of pairs of species, and the homologous CpG sites were identified by applying the Perl module Bio::AlignIO. To remove potential error due to misalignment, the sites were further filtered by requiring the two flanking nucleotides (immediately upstream and downstream each CpG) also being identical for the considered species.

### REFERENCES

1. C. Lopez-Otin, M. A. Blasco, L. Partridge, M. Serrano, G. Kroemer, The hallmarks of aging. Cell 153, 1194-1217 (2013).
2. K. A. Mather, A. F. Jorm, R. A. Parslow, H. Christensen, Is telomere length a biomarker of aging? A review. J Gerontol A Biol Sci Med Sci 66, 202-213 (2011).
3. W. Wagner, Epigenetic aging clocks in mice and men. Genome Biol 18, 107 (2017).
4. G. Hannum et al., Genome-wide methylation profiles reveal quantitative views of human aging rates. Mol Cell 49, 359-367 (2013).
5. S. Horvath, DNA methylation age of human tissues and cell types. Genome Biol 14, R115 (2013).
6. D. A. Petkovich et al., Using DNA Methylation Profiling to Evaluate Biological Age and Longevity Interventions. Cell Metab 25, 954-960 e956 (2017).
7. T. M. Stubbs et al., Multi-tissue DNA methylation age predictor in mouse. Genome Biol 18, 68 (2017).
8. A. Buchwalter, M. W. Hetzer, Nucleolar expansion and elevated protein translation in premature aging. Nat Commun 8, 328 (2017).
9. V. Tiku et al., Small nucleoli are a cellular hallmark of longevity. Nat Commun 8, 16083 (2017).
10. D. A. Sinclair, L. Guarente, Extrachromosomal rDNA circles—a cause of aging in yeast. Cell 91, 1033-1042 (1997).
11. A. R. Ganley, T. Kobayashi, Ribosomal DNA and cellular senescence: new evidence supporting the connection between rDNA and aging. FEMS Yeast Res 14, 49-59 (2014).
12. K. Larson et al., Heterochromatin formation promotes longevity and represses ribosomal RNA synthesis. PLoS Genet 8, e1002473 (2012).
13. R. Santoro, I. Grummt, Molecular mechanisms mediating methylation-dependent silencing of ribosomal gene transcription. Mol Cell 8, 719-725 (2001).
14. B. McStay, I. Grummt, The epigenetics of rRNA genes: from molecular to chromosome biology. Annu Rev Cell Dev Biol 24, 131-157 (2008).
15. H. Zou, T. Hastie, Regularization and variable selection via the elastic net. Journal of the Royal Statistical Society: Series B (Statistical Methodology) 67, 301-320 (2005).
16. K. Swisshelm, C. M. Disteche, J. Thorvaldsen, A. Nelson, D. Salk, Age-related increase in methylation of ribosomal genes and inactivation of chromosome-specific rRNA gene clusters in mouse. Mutat Res 237, 131-146 (1990).
17. O. Hahn et al., Dietary restriction protects from age-associated DNA methylation and induces epigenetic reprogramming of lipid metabolism. Genome Biol 18, 56 (2017).
18. V. K. Rakyan et al., Human aging-associated DNA hypermethylation occurs preferentially at bivalent chromatin domains. Genome Res 20, 434-439 (2010).
19. D. Besser et al., DNA methylation inhibits transcription by RNA polymerase III of a tRNA gene, but not of a 5S rRNA gene. FEBS letters 269, 358-362 (1990).
20. A. R. Vandiver et al., Age and sun exposure-related widespread genomic blocks of hypomethylation in nonmalignant skin. Genome Biol 16, 80 (2015).
21. J. M. Zahn et al., AGEMAP: a gene expression database for aging in mice. PLoS Genet 3, e201 (2007).
22. M. J. Peters et al., The transcriptional landscape of age in human peripheral blood. Nat Commun 6, 8570 (2015).
23. S. Ide, T. Miyazaki, H. Maki, T. Kobayashi, Abundance of ribosomal RNA gene copies maintains genome integrity. Science 327, 693-696 (2010).
24. A. Murayama et al., Epigenetic control of rDNA loci in response to intracellular energy status. Cell 133, 627-639 (2008).
25. I. Grummt, The nucleolus-guardian of cellular homeostasis and genome integrity. Chromosoma 122, 487-497 (2013).
26. M. Thompson, R. A. Haeusler, P. D. Good, D. R. Engelke, Nucleolar clustering of dispersed tRNA genes. Science 302, 1399-1401 (2003).
27. A. Nemeth, G. Langst, Genome organization in and around the nucleolus. Trends Genet 27, 149-156 (2011).
28. J. G. Gibbons, A. T. Branco, S. Yu, B. Lemos, Ribosomal DNA copy number is coupled with gene expression variation and mitochondrial abundance in humans. Nat Commun 5, 4850 (2014).
29. R. Zhao, M. S. Bodnar, D. L. Spector, Nuclear neighborhoods and gene expression. Curr Opin Genet Dev 19, 172-179 (2009).
30. A. H. Michel, B. Kornmann, K. Dubrana, D. Shore, Spontaneous rDNA copy number variation modulates Sir2 levels and epigenetic gene silencing. Genes Dev 19, 1199-1210 (2005).
31. C. A. Sloan et al., ENCODE data at the ENCODE portal. Nucleic Acids Res 44, D726-732 (2016).
32. W. J. Kent, BLAT—the BLAST-like alignment tool. Genome Res 12, 656-664 (2002).
33. F. Krueger, S. R. Andrews, Bismark: a flexible aligner and methylation caller for Bisulfite-Seq applications. Bioinformatics 27, 1571-1572 (2011).
34. B. Langmead, S. L. Salzberg, Fast gapped-read alignment with Bowtie 2. Nature methods 9, 357-359 (2012).
35. J. Friedman, T. Hastie, R. Tibshirani, Regularization Paths for Generalized Linear Models via Coordinate Descent. J Stat Softw 33, 1-22 (2010).
36. B. L. Aken et al., Ensembl 2017. Nucleic Acids Res 45, D635-D642 (2017).
37. C. Tyner et al., The UCSC Genome Browser database: 2017 update. Nucleic Acids Res 45, D626-D634 (2017).
38. P. P. Chan, T. M. Lowe, GtRNAdb 2.0: an expanded database of transfer RNA genes identified in complete and draft genomes. Nucleic Acids Res 44, D184-189 (2016).
39. J. D. Thompson, D. G. Higgins, T. J. Gibson, CLUSTAL W: improving the sensitivity of progressive multiple sequence alignment through sequence weighting, position-specific gap penalties and weight matrix choice. Nucleic Acids Res 22, 4673-4680 (1994).

TABLE 3.
Conserved CpGs between human and mouse.

| Human | | | | Mouse | | | |
|---|---|---|---|---|---|---|---|
| | Location | | | | Location | | |
| Nucleotide | at | | | Nucleotide | at | | |
| Accession | Region | Coordinate | Region | Accession | Region | Coordinate | Context |
| U13369.1 | 379 | 4035 | 18S | BK000964.3 | 380 | 4387 | TCGC |
| U13369.1 | 315 | 3971 | 18S | BK000964.3 | 316 | 4323 | TCGC |
| U13369.1 | 517 | 4173 | 18S | BK000964.3 | 518 | 4525 | ACGA |
| U13369.1 | 840 | 4496 | 18S | BK000964.3 | 841 | 4848 | CCGC |
| U13369.1 | 346 | 4002 | 18S | BK000964.3 | 347 | 4354 | TCGA |
| U13369.1 | 1472 | 5128 | 18S | BK000964.3 | 1472 | 5479 | CCGA |
| U13369.1 | 182 | 3838 | 18S | BK000964.3 | 182 | 4189 | ACGG |
| U13369.1 | 543 | 4199 | 18S | BK000964.3 | 544 | 4551 | TCGA |
| U13369.1 | 1719 | 5375 | 18S | BK000964.3 | 1717 | 5724 | CCGA |
| U13369.1 | 1672 | 5328 | 18S | BK000964.3 | 1670 | 5677 | GCGT |
| U13369.1 | 1749 | 5405 | 18S | BK000964.3 | 1747 | 5754 | TCGG |
| U13369.1 | 1163 | 4819 | 18S | BK000964.3 | 1164 | 5171 | CCGG |
| U13369.1 | 1374 | 5030 | 18S | BK000964.3 | 1374 | 5381 | CCGA |
| U13369.1 | 1085 | 4741 | 18S | BK000964.3 | 1086 | 5093 | ACGA |
| U13369.1 | 974 | 4630 | 18S | BK000964.3 | 975 | 4982 | CCGG |
| U13369.1 | 306 | 3962 | 18S | BK000964.3 | 307 | 4314 | TCGG |
| U13369.1 | 1583 | 5239 | 18S | BK000964.3 | 1583 | 5590 | CCGT |
| U13369.1 | 736 | 4392 | 18S | BK000964.3 | 737 | 4744 | CCGC |
| U13369.1 | 1273 | 4929 | 18S | BK000964.3 | 1273 | 5280 | CCGG |
| U13369.1 | 409 | 4065 | 18S | BK000964.3 | 410 | 4417 | ACGG |
| U13369.1 | 708 | 4364 | 18S | BK000964.3 | 709 | 4716 | CCGC |
| U13369.1 | 186 | 3842 | 18S | BK000964.3 | 186 | 4193 | GCGC |
| U13369.1 | 951 | 4607 | 18S | BK000964.3 | 952 | 4959 | CCGC |
| U13369.1 | 278 | 3934 | 18S | BK000964.3 | 280 | 4287 | CCGG |
| U13369.1 | 585 | 4241 | 18S | BK000964.3 | 586 | 4593 | ACGA |
| U13369.1 | 1710 | 5366 | 18S | BK000964.3 | 1708 | 5715 | TCGC |
| U13369.1 | 1103 | 4759 | 18S | BK000964.3 | 1104 | 5111 | GCGG |
| U13369.1 | 1796 | 5452 | 18S | BK000964.3 | 1794 | 5801 | ACGG |
| U13369.1 | 984 | 4640 | 18S | BK000964.3 | 985 | 4992 | ACGG |
| U13369.1 | 1410 | 5066 | 18S | BK000964.3 | 1410 | 5417 | ACGC |
| U13369.1 | 622 | 4278 | 18S | BK000964.3 | 623 | 4630 | CCGC |
| U13369.1 | 624 | 4280 | 18S | BK000964.3 | 625 | 4632 | GCGG |
| U13369.1 | 69 | 3725 | 18S | BK000964.3 | 69 | 4076 | ACGG |
| U13369.1 | 1525 | 5181 | 18S | BK000964.3 | 1525 | 5532 | GCGC |
| U13369.1 | 1764 | 5420 | 18S | BK000964.3 | 1762 | 5769 | TCGG |
| U13369.1 | 1430 | 5086 | 18S | BK000964.3 | 1430 | 5437 | GCGT |
| U13369.1 | 792 | 4448 | 18S | BK000964.3 | 793 | 4800 | CCGA |
| U13369.1 | 1380 | 5036 | 18S | BK000964.3 | 1380 | 5387 | ACGA |
| U13369.1 | 1039 | 4695 | 18S | BK000964.3 | 1040 | 5047 | TCGG |
| U13369.1 | 879 | 4535 | 18S | BK000964.3 | 880 | 4887 | GCGG |
| U13369.1 | 1523 | 5179 | 18S | BK000964.3 | 1523 | 5530 | ACGC |
| U13369.1 | 1785 | 5441 | 18S | BK000964.3 | 1783 | 5790 | GCGC |
| U13369.1 | 1075 | 4731 | 18S | BK000964.3 | 1076 | 5083 | CCGA |
| U13369.1 | 254 | 3910 | 18S | BK000964.3 | 256 | 4263 | CCGG |
| U13369.1 | 1427 | 5083 | 18S | BK000964.3 | 1427 | 5434 | TCGG |
| U13369.1 | 212 | 3868 | 18S | BK000964.3 | 214 | 4221 | GCGT |
| U13369.1 | 1830 | 5486 | 18S | BK000964.3 | 1828 | 5835 | TCGT |
| U13369.1 | 382 | 4038 | 18S | BK000964.3 | 383 | 4390 | CCGT |
| U13369.1 | 1574 | 5230 | 18S | BK000964.3 | 1574 | 5581 | GCGG |
| U13369.1 | 1091 | 4747 | 18S | BK000964.3 | 1092 | 5099 | CCGA |
| U13369.1 | 1744 | 5400 | 18S | BK000964.3 | 1742 | 5749 | TCGG |
| U13369.1 | 1565 | 5221 | 18S | BK000964.3 | 1565 | 5572 | CCGG |
| U13369.1 | 1572 | 5228 | 18S | BK000964.3 | 1572 | 5579 | GCGC |
| U13369.1 | 1098 | 4754 | 18S | BK000964.3 | 1099 | 5106 | GCGA |
| U13369.1 | 900 | 4556 | 18S | BK000964.3 | 901 | 4908 | TCGG |
| U13369.1 | 498 | 4154 | 18S | BK000964.3 | 499 | 4506 | CCGA |
| U13369.1 | 977 | 4633 | 18S | BK000964.3 | 978 | 4985 | GCGC |
| U13369.1 | 311 | 3967 | 18S | BK000964.3 | 312 | 4319 | CCGA |
| U13369.1 | 676 | 4332 | 18S | BK000964.3 | 677 | 4684 | TCGT |
| U13369.1 | 1780 | 5436 | 18S | BK000964.3 | 1778 | 5785 | GCGG |
| U13369.1 | 797 | 4453 | 18S | BK000964.3 | 798 | 4805 | GCGT |
| U13369.1 | 1311 | 4967 | 18S | BK000964.3 | 1311 | 5318 | TCGA |
| U13369.1 | 1384 | 5040 | 18S | BK000964.3 | 1384 | 5391 | ACGA |
| U13369.1 | 1234 | 4890 | 18S | BK000964.3 | 1234 | 5241 | GCGG |
| U13369.1 | 1609 | 5265 | 18S | BK000964.3 | 1609 | 5616 | TCGG |
| U13369.1 | 450 | 4106 | 18S | BK000964.3 | 451 | 4458 | ACGG |
| U13369.1 | 1597 | 5253 | 18S | BK000964.3 | 1597 | 5604 | TCGT |
| U13369.1 | 1279 | 4935 | 18S | BK000964.3 | 1279 | 5286 | ACGG |
| U13369.1 | 695 | 4351 | 18S | BK000964.3 | 696 | 4703 | GCGG |
| U13369.1 | 1755 | 5411 | 18S | BK000964.3 | 1753 | 5760 | CCGC |
| U13369.1 | 645 | 4301 | 18S | BK000964.3 | 646 | 4653 | GCGT |
| U13369.1 | 1419 | 5075 | 18S | BK000964.3 | 1419 | 5426 | CCGA |
| U13369.1 | 350 | 4006 | 18S | BK000964.3 | 351 | 4358 | ACGT |
| U13369.1 | 785 | 4441 | 18S | BK000964.3 | 786 | 4793 | GCGG |
| U13369.1 | 1067 | 4723 | 18S | BK000964.3 | 1068 | 5075 | TCGT |
| U13369.1 | 1707 | 5363 | 18S | BK000964.3 | 1705 | 5712 | CCGT |
| U13369.1 | 1513 | 5169 | 18S | BK000964.3 | 1513 | 5520 | CCGG |
| U13369.1 | 1047 | 4703 | 18S | BK000964.3 | 1048 | 5055 | TCGA |
| U13369.1 | 1053 | 4709 | 18S | BK000964.3 | 1054 | 5061 | ACGA |
| U13369.1 | 1128 | 4784 | 18S | BK000964.3 | 1129 | 5136 | CCGG |
| U13369.1 | 481 | 4137 | 18S | BK000964.3 | 482 | 4489 | GCGC |
| U13369.1 | 1109 | 4765 | 18S | BK000964.3 | 1110 | 5117 | GCGT |
| U13369.1 | 835 | 4491 | 18S | BK000964.3 | 836 | 4843 | CCGA |
| U13369.1 | 1412 | 5068 | 18S | BK000964.3 | 1412 | 5419 | GCGA |
| U13369.1 | 877 | 4533 | 18S | BK000964.3 | 878 | 4885 | CCGC |
| U13369.1 | 1268 | 4924 | 18S | BK000964.3 | 1268 | 5275 | CCGG |
| U13369.1 | 1423 | 5079 | 18S | BK000964.3 | 1423 | 5430 | GCGG |
| U13369.1 | 337 | 3993 | 18S | BK000964.3 | 338 | 4345 | ACGA |
| U13369.1 | 479 | 4135 | 18S | BK000964.3 | 480 | 4487 | GCGC |
| U13369.1 | 331 | 3987 | 18S | BK000964.3 | 332 | 4339 | GCGG |
| U13369.1 | 1703 | 5359 | 18S | BK000964.3 | 1701 | 5708 | CCGC |
| U13369.1 | 503 | 4159 | 18S | BK000964.3 | 504 | 4511 | CCGG |
| U13369.1 | 931 | 4587 | 18S | BK000964.3 | 932 | 4939 | CCGG |
| U13369.1 | 711 | 4367 | 18S | BK000964.3 | 712 | 4719 | CCGC |
| U13369.1 | 1758 | 5414 | 18S | BK000964.3 | 1756 | 5763 | CCGG |
| U13369.1 | 1355 | 5011 | 18S | BK000964.3 | 1355 | 5362 | GCGA |
| U13369.1 | 1106 | 4762 | 18S | BK000964.3 | 1107 | 5114 | GCGG |
| U13369.1 | 1800 | 5456 | 18S | BK000964.3 | 1798 | 5805 | TCGA |
| U13369.1 | 264 | 3920 | 18S | BK000964.3 | 266 | 4273 | CCGG |
| U13369.1 | 1139 | 4795 | 18S | BK000964.3 | 1140 | 5147 | CCGG |
| U13369.1 | 1527 | 5183 | 18S | BK000964.3 | 1527 | 5534 | GCGC |
| U13369.1 | 1657 | 5313 | 18S | BK000964.3 | 1655 | 5662 | GCGG |
| U13369.1 | 941 | 4597 | 18S | BK000964.3 | 942 | 4949 | TCGT |
| U13369.1 | 1254 | 4910 | 18S | BK000964.3 | 1254 | 5261 | ACGG |
| U13369.1 | 239 | 3895 | 18S | BK000964.3 | 241 | 4248 | CCGG |
| U13369.1 | 1064 | 4720 | 18S | BK000964.3 | 1065 | 5072 | CCGT |
| U13369.1 | 1771 | 5427 | 18S | BK000964.3 | 1769 | 5776 | ACGG |
| U13369.1 | 761 | 4417 | 18S | BK000964.3 | 762 | 4769 | TCGA |
| U13369.1 | 125 | 3781 | 18S | BK000964.3 | 125 | 4132 | TCGC |
| U13369.1 | 1460 | 5116 | 18S | BK000964.3 | 1460 | 5467 | GCGT |
| U13369.1 | 948 | 4604 | 18S | BK000964.3 | 949 | 4956 | GCGC |
| U13369.1 | 726 | 4382 | 18S | BK000964.3 | 727 | 4734 | CCGC |
| U13369.1 | 1205 | 4861 | 18S | BK000964.3 | 1206 | 5213 | ACGG |
| U13369.1 | 275 | 3931 | 18S | BK000964.3 | 277 | 4284 | GCGC |
| U13369.1 | 994 | 4650 | 18S | BK000964.3 | 995 | 5002 | GCGA |
| U13369.1 | 783 | 4439 | 18S | BK000964.3 | 784 | 4791 | CCGC |
| U13369.1 | 89 | 3745 | 18S | BK000964.3 | 89 | 4096 | GCGA |
| U13369.1 | 1317 | 4973 | 18S | BK000964.3 | 1317 | 5324 | CCGT |
| U13369.1 | 851 | 4507 | 18S | BK000964.3 | 852 | 4859 | CCGC |
| U13369.1 | 430 | 4086 | 18S | BK000964.3 | 431 | 4438 | CCGG |
| U13369.1 | 326 | 3982 | 18S | BK000964.3 | 327 | 4334 | CCGT |
| U13369.1 | 1337 | 4993 | 18S | BK000964.3 | 1337 | 5344 | CCGT |
| U13369.1 | 369 | 4025 | 18S | BK000964.3 | 370 | 4377 | TCGA |
| U13369.1 | 73 | 3729 | 18S | BK000964.3 | 73 | 4080 | CCGG |
| U13369.1 | 334 | 3990 | 18S | BK000964.3 | 335 | 4342 | GCGA |
| U13369.1 | 730 | 4386 | 18S | BK000964.3 | 731 | 4738 | CCGT |
| U13369.1 | 753 | 4409 | 18S | BK000964.3 | 754 | 4761 | GCGC |
| U13369.1 | 750 | 4406 | 18S | BK000964.3 | 751 | 4758 | TCGG |
| U13369.1 | 1844 | 5500 | 18S | BK000964.3 | 1842 | 5849 | CCGT |
| U13369.1 | 179 | 3835 | 18S | BK000964.3 | 179 | 4186 | CCGA |
| U13369.1 | 281 | 3937 | 18S | BK000964.3 | 283 | 4290 | GCGG |
| U13369.1 | 927 | 4583 | 18S | BK000964.3 | 928 | 4935 | ACGG |
| U13369.1 | 1635 | 5291 | 18S | BK000964.3 | 1635 | 5642 | ACGA |
| U13369.1 | 718 | 4374 | 18S | BK000964.3 | 719 | 4726 | GCGA |
| U13369.1 | 260 | 3916 | 18S | BK000964.3 | 262 | 4269 | CCGG |
| U13369.1 | 319 | 3975 | 18S | BK000964.3 | 320 | 4327 | ACGC |
| U13369.1 | 713 | 4369 | 18S | BK000964.3 | 714 | 4721 | GCGA |
| U13369.1 | 1032 | 4688 | 18S | BK000964.3 | 1033 | 5040 | ACGA |
| U13369.1 | 402 | 4058 | 18S | BK000964.3 | 403 | 4410 | ACGG |
| U13369.1 | 1858 | 5514 | 18S | BK000964.3 | 1856 | 5863 | GCGG |
| U13369.1 | 699 | 4355 | 18S | BK000964.3 | 700 | 4707 | GCGG |
| U13369.1 | 424 | 4080 | 18S | BK000964.3 | 425 | 4432 | TCGA |
| U13369.1 | 703 | 4359 | 18S | BK000964.3 | 704 | 4711 | GCGG |
| U13369.1 | 1125 | 4781 | 18S | BK000964.3 | 1126 | 5133 | CCGC |
| U13369.1 | 1547 | 5203 | 18S | BK000964.3 | 1547 | 5554 | GCGT |
| U13369.1 | 129 | 3785 | 18S | BK000964.3 | 129 | 4136 | TCGC |
| U13369.1 | 65 | 3721 | 18S | BK000964.3 | 65 | 4072 | ACGC |
| U13369.1 | 105 | 6727 | 5.8S | BK000964.3 | 104 | 6981 | ACGC |
| U13369.1 | 11 | 6633 | 5.8S | BK000964.3 | 10 | 6887 | GCGG |
| U13369.1 | 35 | 6657 | 5.8S | BK000964.3 | 34 | 6911 | TCGA |
| U13369.1 | 93 | 6715 | 5.8S | BK000964.3 | 92 | 6969 | TCGA |
| U13369.1 | 28 | 6650 | 5.8S | BK000964.3 | 27 | 6904 | TCGT |
| U13369.1 | 32 | 6654 | 5.8S | BK000964.3 | 31 | 6908 | GCGT |
| U13369.1 | 113 | 6735 | 5.8S | BK000964.3 | 112 | 6989 | GCGG |
| U13369.1 | 101 | 6723 | 5.8S | BK000964.3 | 100 | 6977 | TCGA |
| U13369.1 | 23 | 6645 | 5.8S | BK000964.3 | 22 | 6899 | TCGG |
| U13369.1 | 130 | 6752 | 5.8S | BK000964.3 | 129 | 7006 | CCGG |
| U13369.1 | 57 | 6679 | 5.8S | BK000964.3 | 56 | 6933 | GCGA |
| U13369.1 | 45 | 6667 | 5.8S | BK000964.3 | 44 | 6921 | ACGC |
| U13369.1 | 119 | 6741 | 5.8S | BK000964.3 | 118 | 6995 | CCGG |
| U13369.1 | 138 | 6760 | 5.8S | BK000964.3 | 137 | 7014 | ACGC |
| U13369.1 | 150 | 6772 | 5.8S | BK000964.3 | 149 | 7026 | GCGT |
| U13369.1 | 2465 | 10399 | 28S | BK000964.3 | 2233 | 10355 | ACGG |
| U13369.1 | 2638 | 10572 | 28S | BK000964.3 | 2404 | 10526 | GCGC |
| U13369.1 | 3639 | 11573 | 28S | BK000964.3 | 3316 | 11438 | GCGA |
| U13369.1 | 4100 | 12034 | 28S | BK000964.3 | 3773 | 11895 | GCGC |
| U13369.1 | 3265 | 11199 | 28S | BK000964.3 | 3030 | 11152 | GCGG |
| U13369.1 | 267 | 8201 | 28S | BK000964.3 | 275 | 8397 | CCGG |
| U13369.1 | 1371 | 9305 | 28S | BK000964.3 | 1198 | 9320 | GCGC |
| U13369.1 | 1572 | 9506 | 28S | BK000964.3 | 1398 | 9520 | GCGG |
| U13369.1 | 427 | 8361 | 28S | BK000964.3 | 435 | 8557 | ACGG |
| U13369.1 | 3662 | 11596 | 28S | BK000964.3 | 3339 | 11461 | ACGC |
| U13369.1 | 3207 | 11141 | 28S | BK000964.3 | 2972 | 11094 | GCGG |
| U13369.1 | 690 | 8624 | 28S | BK000964.3 | 706 | 8828 | GCGG |
| U13369.1 | 2840 | 10774 | 28S | BK000964.3 | 2606 | 10728 | TCGG |
| U13369.1 | 2854 | 10788 | 28S | BK000964.3 | 2620 | 10742 | CCGT |
| U13369.1 | 2762 | 10696 | 28S | BK000964.3 | 2528 | 10650 | CCGG |
| U13369.1 | 4489 | 12423 | 28S | BK000964.3 | 4171 | 12293 | ACGT |
| U13369.1 | 4973 | 12907 | 28S | BK000964.3 | 4655 | 12777 | ACGT |
| U13369.1 | 1871 | 9805 | 28S | BK000964.3 | 1686 | 9808 | GCGG |
| U13369.1 | 1418 | 9352 | 28S | BK000964.3 | 1244 | 9366 | CCGA |
| U13369.1 | 941 | 8875 | 28S | BK000964.3 | 851 | 8973 | CCGA |
| U13369.1 | 2689 | 10623 | 28S | BK000964.3 | 2455 | 10577 | TCGC |
| U13369.1 | 2930 | 10864 | 28S | BK000964.3 | 2696 | 10818 | CCGC |
| U13369.1 | 4016 | 11950 | 28S | BK000964.3 | 3688 | 11810 | CCGC |
| U13369.1 | 2322 | 10256 | 28S | BK000964.3 | 2090 | 10212 | CCGC |
| U13369.1 | 698 | 8632 | 28S | BK000964.3 | 714 | 8836 | CCGC |
| U13369.1 | 4417 | 12351 | 28S | BK000964.3 | 4099 | 12221 | TCGA |
| U13369.1 | 3805 | 11739 | 28S | BK000964.3 | 3482 | 11604 | ACGA |
| U13369.1 | 4185 | 12119 | 28S | BK000964.3 | 3867 | 11989 | ACGG |
| U13369.1 | 1819 | 9753 | 28S | BK000964.3 | 1634 | 9756 | GCGT |
| U13369.1 | 1363 | 9297 | 28S | BK000964.3 | 1190 | 9312 | CCGC |
| U13369.1 | 1263 | 9197 | 28S | BK000964.3 | 1090 | 9212 | GCGC |
| U13369.1 | 3226 | 11160 | 28S | BK000964.3 | 2991 | 11113 | ACGC |
| U13369.1 | 962 | 8896 | 28S | BK000964.3 | 874 | 8996 | CCGC |
| U13369.1 | 1508 | 9442 | 28S | BK000964.3 | 1334 | 9456 | CCGA |
| U13369.1 | 3233 | 11167 | 28S | BK000964.3 | 2998 | 11120 | CCGG |
| U13369.1 | 1197 | 9131 | 28S | BK000964.3 | 1043 | 9165 | TCGG |
| U13369.1 | 1750 | 9684 | 28S | BK000964.3 | 1565 | 9687 | CCGA |
| U13369.1 | 2697 | 10631 | 28S | BK000964.3 | 2463 | 10585 | CCGG |
| U13369.1 | 4510 | 12444 | 28S | BK000964.3 | 4192 | 12314 | TCGT |
| U13369.1 | 710 | 8644 | 28S | BK000964.3 | 726 | 8848 | CCGG |
| U13369.1 | 4058 | 11992 | 28S | BK000964.3 | 3730 | 11852 | CCGG |
| U13369.1 | 2479 | 10413 | 28S | BK000964.3 | 2247 | 10369 | CCGT |
| U13369.1 | 2364 | 10298 | 28S | BK000964.3 | 2132 | 10254 | ACGA |
| U13369.1 | 2181 | 10115 | 28S | BK000964.3 | 1968 | 10090 | CCGC |
| U13369.1 | 583 | 8517 | 28S | BK000964.3 | 595 | 8717 | GCGG |
| U13369.1 | 4895 | 12829 | 28S | BK000964.3 | 4575 | 12697 | CCGT |
| U13369.1 | 3720 | 11654 | 28S | BK000964.3 | 3397 | 11519 | GCGG |
| U13369.1 | 2572 | 10506 | 28S | BK000964.3 | 2337 | 10459 | GCGA |
| U13369.1 | 2940 | 10874 | 28S | BK000964.3 | 2706 | 10828 | ACGA |
| U13369.1 | 537 | 8471 | 28S | BK000964.3 | 549 | 8671 | CCGC |
| U13369.1 | 2259 | 10193 | 28S | BK000964.3 | 2027 | 10149 | CCGC |
| U13369.1 | 156 | 8090 | 28S | BK000964.3 | 159 | 8281 | ACGG |
| U13369.1 | 4042 | 11976 | 28S | BK000964.3 | 3714 | 11836 | TCGT |
| U13369.1 | 619 | 8553 | 28S | BK000964.3 | 636 | 8758 | CCGG |
| U13369.1 | 1084 | 9018 | 28S | BK000964.3 | 952 | 9074 | CCGC |
| U13369.1 | 4066 | 12000 | 28S | BK000964.3 | 3738 | 11860 | GCGG |
| U13369.1 | 2256 | 10190 | 28S | BK000964.3 | 2024 | 10146 | ACGC |
| U13369.1 | 2068 | 10002 | 28S | BK000964.3 | 1883 | 10005 | TCGC |
| U13369.1 | 894 | 8828 | 28S | BK000964.3 | 806 | 8928 | CCGA |
| U13369.1 | 3597 | 11531 | 28S | BK000964.3 | 3274 | 11396 | GCGG |
| U13369.1 | 1078 | 9012 | 28S | BK000964.3 | 946 | 9068 | GCGC |
| U13369.1 | 4808 | 12742 | 28S | BK000964.3 | 4488 | 12610 | CCGC |
| U13369.1 | 4125 | 12059 | 28S | BK000964.3 | 3807 | 11929 | GCGA |
| U13369.1 | 2582 | 10516 | 28S | BK000964.3 | 2347 | 10469 | CCGG |
| U13369.1 | 4705 | 12639 | 28S | BK000964.3 | 4388 | 12510 | GCGC |
| U13369.1 | 3581 | 11515 | 28S | BK000964.3 | 3258 | 11380 | CCGA |
| U13369.1 | 2535 | 10469 | 28S | BK000964.3 | 2303 | 10425 | GCGG |
| U13369.1 | 1691 | 9625 | 28S | BK000964.3 | 1515 | 9637 | CCGA |
| U13369.1 | 4573 | 12507 | 28S | BK000964.3 | 4255 | 12377 | ACGA |
| U13369.1 | 1881 | 9815 | 28S | BK000964.3 | 1696 | 9818 | CCGA |
| U13369.1 | 4939 | 12873 | 28S | BK000964.3 | 4621 | 12743 | TCGT |
| U13369.1 | 5019 | 12953 | 28S | BK000964.3 | 4701 | 12823 | TCGA |
| U13369.1 | 2147 | 10081 | 28S | BK000964.3 | 1953 | 10075 | GCGG |
| U13369.1 | 2325 | 10259 | 28S | BK000964.3 | 2093 | 10215 | CCGC |
| U13369.1 | 2570 | 10504 | 28S | BK000964.3 | 2335 | 10457 | ACGC |
| U13369.1 | 4927 | 12861 | 28S | BK000964.3 | 4609 | 12731 | GCGC |
| U13369.1 | 1443 | 9377 | 28S | BK000964.3 | 1269 | 9391 | GCGC |
| U13369.1 | 4207 | 12141 | 28S | BK000964.3 | 3889 | 12011 | GCGA |
| U13369.1 | 2488 | 10422 | 28S | BK000964.3 | 2256 | 10378 | TCGG |
| U13369.1 | 4883 | 12817 | 28S | BK000964.3 | 4563 | 12685 | CCGC |
| U13369.1 | 2282 | 10216 | 28S | BK000964.3 | 2050 | 10172 | GCGG |
| U13369.1 | 3787 | 11721 | 28S | BK000964.3 | 3464 | 11586 | ACGC |
| U13369.1 | 1958 | 9892 | 28S | BK000964.3 | 1773 | 9895 | ACGG |
| U13369.1 | 3770 | 11704 | 28S | BK000964.3 | 3447 | 11569 | TCGT |
| U13369.1 | 1189 | 9123 | 28S | BK000964.3 | 1035 | 9157 | TCGC |
| U13369.1 | 3244 | 11178 | 28S | BK000964.3 | 3009 | 11131 | CCGT |
| U13369.1 | 2767 | 10701 | 28S | BK000964.3 | 2533 | 10655 | CCGT |
| U13369.1 | 3541 | 11475 | 28S | BK000964.3 | 3214 | 11336 | GCGG |
| U13369.1 | 4913 | 12847 | 28S | BK000964.3 | 4595 | 12717 | TCGT |
| U13369.1 | 1059 | 8993 | 28S | BK000964.3 | 927 | 9049 | CCGG |
| U13369.1 | 823 | 8757 | 28S | BK000964.3 | 786 | 8908 | GCGC |
| U13369.1 | 2088 | 10022 | 28S | BK000964.3 | 1902 | 10024 | ACGG |
| U13369.1 | 225 | 8159 | 28S | BK000964.3 | 228 | 8350 | ACGG |
| U13369.1 | 2049 | 9983 | 28S | BK000964.3 | 1864 | 9986 | TCGG |
| U13369.1 | 1456 | 9390 | 28S | BK000964.3 | 1282 | 9404 | CCGT |
| U13369.1 | 4891 | 12825 | 28S | BK000964.3 | 4571 | 12693 | TCGC |
| U13369.1 | 4961 | 12895 | 28S | BK000964.3 | 4643 | 12765 | TCGG |
| U13369.1 | 4997 | 12931 | 28S | BK000964.3 | 4679 | 12801 | GCGA |
| U13369.1 | 3539 | 11473 | 28S | BK000964.3 | 3212 | 11334 | CCGC |
| U13369.1 | 2550 | 10484 | 28S | BK000964.3 | 2318 | 10440 | GCGA |
| U13369.1 | 4379 | 12313 | 28S | BK000964.3 | 4061 | 12183 | GCGG |
| U13369.1 | 4289 | 12223 | 28S | BK000964.3 | 3971 | 12093 | CCGT |
| U13369.1 | 2548 | 10482 | 28S | BK000964.3 | 2316 | 10438 | CCGC |
| U13369.1 | 3272 | 11206 | 28S | BK000964.3 | 3037 | 11159 | TCGC |
| U13369.1 | 4871 | 12805 | 28S | BK000964.3 | 4551 | 12673 | CCGG |
| U13369.1 | 3 | 7937 | 28S | BK000964.3 | 3 | 8125 | GCGA |
| U13369.1 | 4191 | 12125 | 28S | BK000964.3 | 3873 | 11995 | ACGC |
| U13369.1 | 1276 | 9210 | 28S | BK000964.3 | 1103 | 9225 | GCGG |
| U13369.1 | 2247 | 10181 | 28S | BK000964.3 | 2015 | 10137 | GCGG |
| U13369.1 | 508 | 8442 | 28S | BK000964.3 | 520 | 8642 | CCGC |
| U13369.1 | 2927 | 10861 | 28S | BK000964.3 | 2693 | 10815 | GCGC |
| U13369.1 | 3296 | 11230 | 28S | BK000964.3 | 3061 | 11183 | GCGG |
| U13369.1 | 2527 | 10461 | 28S | BK000964.3 | 2295 | 10417 | CCGG |
| U13369.1 | 2312 | 10246 | 28S | BK000964.3 | 2080 | 10202 | CCGG |
| U13369.1 | 182 | 8116 | 28S | BK000964.3 | 185 | 8307 | TCGT |
| U13369.1 | 3664 | 11598 | 28S | BK000964.3 | 3341 | 11463 | GCGA |
| U13369.1 | 3251 | 11185 | 28S | BK000964.3 | 3016 | 11138 | TCGC |
| U13369.1 | 2925 | 10859 | 28S | BK000964.3 | 2691 | 10813 | GCGC |
| U13369.1 | 1273 | 9207 | 28S | BK000964.3 | 1100 | 9222 | TCGG |
| U13369.1 | 273 | 8207 | 28S | BK000964.3 | 281 | 8403 | TCGG |
| U13369.1 | 497 | 8431 | 28S | BK000964.3 | 509 | 8631 | GCGG |
| U13369.1 | 2892 | 10826 | 28S | BK000964.3 | 2658 | 10780 | TCGG |
| U13369.1 | 23 | 7957 | 28S | BK000964.3 | 23 | 8145 | GCGA |
| U13369.1 | 1581 | 9515 | 28S | BK000964.3 | 1407 | 9529 | ACGT |
| U13369.1 | 4824 | 12758 | 28S | BK000964.3 | 4504 | 12626 | CCGG |
| U13369.1 | 2700 | 10634 | 28S | BK000964.3 | 2466 | 10588 | GCGG |
| U13369.1 | 481 | 8415 | 28S | BK000964.3 | 491 | 8613 | CCGT |
| U13369.1 | 4699 | 12633 | 28S | BK000964.3 | 4382 | 12504 | ACGG |
| U13369.1 | 3837 | 11771 | 28S | BK000964.3 | 3514 | 11636 | GCGA |
| U13369.1 | 249 | 8183 | 28S | BK000964.3 | 254 | 8376 | GCGC |
| U13369.1 | 2564 | 10498 | 28S | BK000964.3 | 2329 | 10451 | GCGG |
| U13369.1 | 1239 | 9173 | 28S | BK000964.3 | 1077 | 9199 | CCGA |
| U13369.1 | 660 | 8594 | 28S | BK000964.3 | 676 | 8798 | CCGG |
| U13369.1 | 2304 | 10238 | 28S | BK000964.3 | 2072 | 10194 | GCGC |
| U13369.1 | 2469 | 10403 | 28S | BK000964.3 | 2237 | 10359 | GCGA |
| U13369.1 | 4019 | 11953 | 28S | BK000964.3 | 3691 | 11813 | CCGG |
| U13369.1 | 3551 | 11485 | 28S | BK000964.3 | 3224 | 11346 | GCGC |
| U13369.1 | 1297 | 9231 | 28S | BK000964.3 | 1124 | 9246 | CCGA |
| U13369.1 | 2061 | 9995 | 28S | BK000964.3 | 1876 | 9998 | CCGG |
| U13369.1 | 1191 | 9125 | 28S | BK000964.3 | 1037 | 9159 | GCGC |
| U13369.1 | 2264 | 10198 | 28S | BK000964.3 | 2032 | 10154 | ACGA |
| U13369.1 | 1815 | 9749 | 28S | BK000964.3 | 1630 | 9752 | CCGG |
| U13369.1 | 746 | 8680 | 28S | BK000964.3 | 762 | 8884 | TCGG |
| U13369.1 | 1302 | 9236 | 28S | BK000964.3 | 1129 | 9251 | CCGT |
| U13369.1 | 3278 | 11212 | 28S | BK000964.3 | 3043 | 11165 | CCGC |
| U13369.1 | 1405 | 9339 | 28S | BK000964.3 | 1231 | 9353 | CCGA |
| U13369.1 | 4945 | 12879 | 28S | BK000964.3 | 4627 | 12749 | ACGA |
| U13369.1 | 405 | 8339 | 28S | BK000964.3 | 413 | 8535 | GCGT |
| U13369.1 | 3857 | 11791 | 28S | BK000964.3 | 3534 | 11656 | ACGG |
| U13369.1 | 2591 | 10525 | 28S | BK000964.3 | 2356 | 10478 | CCGG |
| U13369.1 | 1267 | 9201 | 28S | BK000964.3 | 1094 | 9216 | ACGG |
| U13369.1 | 2134 | 10068 | 28S | BK000964.3 | 1940 | 10062 | TCGG |
| U13369.1 | 4507 | 12441 | 28S | BK000964.3 | 4189 | 12311 | CCGT |
| U13369.1 | 2507 | 10441 | 28S | BK000964.3 | 2275 | 10397 | TCGG |
| U13369.1 | 175 | 8109 | 28S | BK000964.3 | 178 | 8300 | GCGC |
| U13369.1 | 2923 | 10857 | 28S | BK000964.3 | 2689 | 10811 | GCGC |
| U13369.1 | 251 | 8185 | 28S | BK000964.3 | 256 | 8378 | GCGC |
| U13369.1 | 1755 | 9689 | 28S | BK000964.3 | 1570 | 9692 | ACGA |
| U13369.1 | 959 | 8893 | 28S | BK000964.3 | 871 | 8993 | TCGC |
| U13369.1 | 4387 | 12321 | 28S | BK000964.3 | 4069 | 12191 | GCGT |
| U13369.1 | 3284 | 11218 | 28S | BK000964.3 | 3049 | 11171 | CCGG |
| U13369.1 | 4396 | 12330 | 28S | BK000964.3 | 4078 | 12200 | GCGA |
| U13369.1 | 4256 | 12190 | 28S | BK000964.3 | 3938 | 12060 | TCGC |
| U13369.1 | 4297 | 12231 | 28S | BK000964.3 | 3979 | 12101 | GCGG |
| U13369.1 | 4718 | 12652 | 28S | BK000964.3 | 4402 | 12524 | TCGG |
| U13369.1 | 3307 | 11241 | 28S | BK000964.3 | 3071 | 11193 | GCGC |
| U13369.1 | 4992 | 12926 | 28S | BK000964.3 | 4674 | 12796 | TCGC |
| U13369.1 | 1366 | 9300 | 28S | BK000964.3 | 1193 | 9315 | CCGT |
| U13369.1 | 28 | 7962 | 28S | BK000964.3 | 28 | 8150 | CCGC |
| U13369.1 | 300 | 8234 | 28S | BK000964.3 | 308 | 8430 | GCGG |
| U13369.1 | 4736 | 12670 | 28S | BK000964.3 | 4420 | 12542 | CCGG |
| U13369.1 | 3204 | 11138 | 28S | BK000964.3 | 2969 | 11091 | CCGG |
| U13369.1 | 4663 | 12597 | 28S | BK000964.3 | 4345 | 12467 | ACGC |
| U13369.1 | 3274 | 11208 | 28S | BK000964.3 | 3039 | 11161 | GCGG |
| U13369.1 | 3652 | 11586 | 28S | BK000964.3 | 3329 | 11451 | GCGG |
| U13369.1 | 4278 | 12212 | 28S | BK000964.3 | 3960 | 12082 | ACGA |
| U13369.1 | 413 | 8347 | 28S | BK000964.3 | 421 | 8543 | CCGT |
| U13369.1 | 2906 | 10840 | 28S | BK000964.3 | 2672 | 10794 | GCGC |
| U13369.1 | 2862 | 10796 | 28S | BK000964.3 | 2628 | 10750 | TCGG |
| U13369.1 | 3521 | 11455 | 28S | BK000964.3 | 3194 | 11316 | CCGG |
| U13369.1 | 928 | 8862 | 28S | BK000964.3 | 838 | 8960 | CCGA |
| U13369.1 | 1075 | 9009 | 28S | BK000964.3 | 943 | 9065 | ACGG |
| U13369.1 | 3731 | 11665 | 28S | BK000964.3 | 3408 | 11530 | GCGG |
| U13369.1 | 4423 | 12357 | 28S | BK000964.3 | 4105 | 12227 | TCGG |
| U13369.1 | 1627 | 9561 | 28S | BK000964.3 | 1453 | 9575 | TCGA |
| U13369.1 | 1807 | 9741 | 28S | BK000964.3 | 1622 | 9744 | GCGT |
| U13369.1 | 4307 | 12241 | 28S | BK000964.3 | 3989 | 12111 | ACGA |
| U13369.1 | 2457 | 10391 | 28S | BK000964.3 | 2225 | 10347 | CCGA |
| U13369.1 | 1488 | 9422 | 28S | BK000964.3 | 1314 | 9436 | ACGA |
| U13369.1 | 447 | 8381 | 28S | BK000964.3 | 455 | 8577 | CCGC |
| U13369.1 | 2046 | 9980 | 28S | BK000964.3 | 1861 | 9983 | GCGT |
| U13369.1 | 4399 | 12333 | 28S | BK000964.3 | 4081 | 12203 | ACGT |
| U13369.1 | 956 | 8890 | 28S | BK000964.3 | 868 | 8990 | CCGT |
| U13369.1 | 2306 | 10240 | 28S | BK000964.3 | 2074 | 10196 | GCGG |
| U13369.1 | 2654 | 10588 | 28S | BK000964.3 | 2420 | 10542 | TCGC |
| U13369.1 | 4091 | 12025 | 28S | BK000964.3 | 3764 | 11886 | TCGC |
| U13369.1 | 2103 | 10037 | 28S | BK000964.3 | 1913 | 10035 | GCGG |
| U13369.1 | 1154 | 9088 | 28S | BK000964.3 | 999 | 9121 | GCGG |
| U13369.1 | 715 | 8649 | 28S | BK000964.3 | 731 | 8853 | ACGG |
| U13369.1 | 2038 | 9972 | 28S | BK000964.3 | 1853 | 9975 | GCGC |
| U13369.1 | 3567 | 11501 | 28S | BK000964.3 | 3244 | 11366 | CCGG |
| U13369.1 | 3612 | 11546 | 28S | BK000964.3 | 3289 | 11411 | CCGA |
| U13369.1 | 4460 | 12394 | 28S | BK000964.3 | 4142 | 12264 | GCGT |
| U13369.1 | 524 | 8458 | 28S | BK000964.3 | 536 | 8658 | CCGA |
| U13369.1 | 2896 | 10830 | 28S | BK000964.3 | 2662 | 10784 | TCGG |
| U13369.1 | 1885 | 9819 | 28S | BK000964.3 | 1700 | 9822 | ACGC |
| U13369.1 | 4836 | 12770 | 28S | BK000964.3 | 4516 | 12638 | GCGG |
| U13369.1 | 2594 | 10528 | 28S | BK000964.3 | 2359 | 10481 | GCGG |
| U13369.1 | 3647 | 11581 | 28S | BK000964.3 | 3324 | 11446 | CCGC |
| U13369.1 | 1615 | 9549 | 28S | BK000964.3 | 1441 | 9563 | GCGA |
| U13369.1 | 440 | 8374 | 28S | BK000964.3 | 448 | 8570 | GCGC |
| U13369.1 | 825 | 8759 | 28S | BK000964.3 | 788 | 8910 | GCGC |
| U13369.1 | 3570 | 11504 | 28S | BK000964.3 | 3247 | 11369 | GCGC |
| U13369.1 | 4682 | 12616 | 28S | BK000964.3 | 4364 | 12486 | CCGC |
| U13369.1 | 219 | 8153 | 28S | BK000964.3 | 222 | 8344 | CCGT |
| U13369.1 | 1898 | 9832 | 28S | BK000964.3 | 1713 | 9835 | GCGC |
| U13369.1 | 4402 | 12336 | 28S | BK000964.3 | 4084 | 12206 | TCGC |
| U13369.1 | 342 | 8276 | 28S | BK000964.3 | 350 | 8472 | CCGA |
| U13369.1 | 4169 | 12103 | 28S | BK000964.3 | 3851 | 11973 | GCGG |
| U13369.1 | 465 | 8399 | 28S | BK000964.3 | 473 | 8595 | CCGG |
| U13369.1 | 2120 | 10054 | 28S | BK000964.3 | 1927 | 10049 | GCGT |
| U13369.1 | 53 | 7987 | 28S | BK000964.3 | 53 | 8175 | GCGG |
| U13369.1 | 1911 | 9845 | 28S | BK000964.3 | 1726 | 9848 | ACGC |
| U13369.1 | 477 | 8411 | 28S | BK000964.3 | 487 | 8609 | CCGG |
| U13369.1 | 2428 | 10362 | 28S | BK000964.3 | 2196 | 10318 | TCGG |
| U13369.1 | 2945 | 10879 | 28S | BK000964.3 | 2711 | 10833 | GCGC |
| U13369.1 | 451 | 8385 | 28S | BK000964.3 | 459 | 8581 | CCGG |
| U13369.1 | 1434 | 9368 | 28S | BK000964.3 | 1260 | 9382 | CCGC |
| U13369.1 | 2686 | 10620 | 28S | BK000964.3 | 2452 | 10574 | GCGT |
| U13369.1 | 2736 | 10670 | 28S | BK000964.3 | 2502 | 10624 | CCGG |
| U13369.1 | 1159 | 9093 | 28S | BK000964.3 | 1004 | 9126 | GCGG |
| U13369.1 | 2759 | 10693 | 28S | BK000964.3 | 2525 | 10647 | GCGC |
| U13369.1 | 3262 | 11196 | 28S | BK000964.3 | 3027 | 11149 | GCGG |
| U13369.1 | 3875 | 11809 | 28S | BK000964.3 | 3552 | 11674 | GCGG |
| U13369.1 | 4011 | 11945 | 28S | BK000964.3 | 3683 | 11805 | GCGG |
| U13369.1 | 2245 | 10179 | 28S | BK000964.3 | 2013 | 10135 | CCGC |
| U13369.1 | 438 | 8372 | 28S | BK000964.3 | 446 | 8568 | CCGC |
| U13369.1 | 3148 | 11082 | 28S | BK000964.3 | 2911 | 11033 | CCGG |
| U13369.1 | 3637 | 11571 | 28S | BK000964.3 | 3314 | 11436 | TCGC |
| U13369.1 | 4632 | 12566 | 28S | BK000964.3 | 4314 | 12436 | GCGA |
| U13369.1 | 359 | 8293 | 28S | BK000964.3 | 367 | 8489 | CCGT |
| U13369.1 | 1314 | 9248 | 28S | BK000964.3 | 1141 | 9263 | ACGG |
| U13369.1 | 2691 | 10625 | 28S | BK000964.3 | 2457 | 10579 | GCGG |
| U13369.1 | 172 | 8106 | 28S | BK000964.3 | 175 | 8297 | CCGG |
| U13369.1 | 935 | 8869 | 28S | BK000964.3 | 845 | 8967 | CCGG |
| U13369.1 | 242 | 8176 | 28S | BK000964.3 | 245 | 8367 | GCGG |
| U13369.1 | 674 | 8608 | 28S | BK000964.3 | 690 | 8812 | GCGC |
| U13369.1 | 236 | 8170 | 28S | BK000964.3 | 239 | 8361 | CCGG |
| U13369.1 | 4130 | 12064 | 28S | BK000964.3 | 3812 | 11934 | CCGC |
| U13369.1 | 3563 | 11497 | 28S | BK000964.3 | 3240 | 11362 | TCGG |
| U13369.1 | 2065 | 9999 | 28S | BK000964.3 | 1880 | 10002 | CCGT |
| U13369.1 | 2659 | 10593 | 28S | BK000964.3 | 2425 | 10547 | CCGA |
| U13369.1 | 1337 | 9271 | 28S | BK000964.3 | 1164 | 9286 | GCGC |
| U13369.1 | 704 | 8638 | 28S | BK000964.3 | 720 | 8842 | CCGG |
| U13369.1 | 92 | 8026 | 28S | BK000964.3 | 92 | 8214 | GCGA |
| U13369.1 | 2707 | 10641 | 28S | BK000964.3 | 2473 | 10595 | CCGG |
| U13369.1 | 2521 | 10455 | 28S | BK000964.3 | 2289 | 10411 | CCGA |
| U13369.1 | 1801 | 9735 | 28S | BK000964.3 | 1616 | 9738 | TCGC |
| U13369.1 | 1828 | 9762 | 28S | BK000964.3 | 1643 | 9765 | GCGA |
| U13369.1 | 1437 | 9371 | 28S | BK000964.3 | 1263 | 9385 | CCGA |
| U13369.1 | 2261 | 10195 | 28S | BK000964.3 | 2029 | 10151 | GCGA |
| U13369.1 | 494 | 8428 | 28S | BK000964.3 | 506 | 8628 | CCGG |
| U13369.1 | 3916 | 11850 | 28S | BK000964.3 | 3593 | 11715 | ACGG |
| U13369.1 | 2779 | 10713 | 28S | BK000964.3 | 2545 | 10667 | CCGC |
| U13369.1 | 2964 | 10898 | 28S | BK000964.3 | 2733 | 10855 | CCGG |
| U13369.1 | 634 | 8568 | 28S | BK000964.3 | 651 | 8773 | GCGG |
| U13369.1 | 1594 | 9528 | 28S | BK000964.3 | 1420 | 9542 | TCGT |
| U13369.1 | 2672 | 10606 | 28S | BK000964.3 | 2438 | 10560 | CCGT |
| U13369.1 | 695 | 8629 | 28S | BK000964.3 | 711 | 8833 | GCGC |
| U13369.1 | 1567 | 9501 | 28S | BK000964.3 | 1393 | 9515 | CCGT |
| U13369.1 | 4074 | 12008 | 28S | BK000964.3 | 3746 | 11868 | GCGA |
| U13369.1 | 3518 | 11452 | 28S | BK000964.3 | 3191 | 11313 | CCGC |
| U13369.1 | 4694 | 12628 | 28S | BK000964.3 | 4377 | 12499 | ACGA |
| U13369.1 | 4760 | 12694 | 28S | BK000964.3 | 4445 | 12567 | GCGG |
| U13369.1 | 1888 | 9822 | 28S | BK000964.3 | 1703 | 9825 | CCGG |
| U13369.1 | 3376 | 11310 | 28S | BK000964.3 | 3133 | 11255 | GCGG |
| U13369.1 | 1451 | 9385 | 28S | BK000964.3 | 1277 | 9399 | CCGG |
| U13369.1 | 126 | 8060 | 28S | BK000964.3 | 126 | 8248 | CCGC |
| U13369.1 | 4583 | 12517 | 28S | BK000964.3 | 4265 | 12387 | CCGC |
| U13369.1 | 656 | 8590 | 28S | BK000964.3 | 672 | 8794 | GCGA |
| U13369.1 | 336 | 8270 | 28S | BK000964.3 | 344 | 8466 | ACGA |
| U13369.1 | 1981 | 9915 | 28S | BK000964.3 | 1796 | 9918 | CCGC |
| U13369.1 | 2545 | 10479 | 28S | BK000964.3 | 2313 | 10435 | GCGC |
| U13369.1 | 2848 | 10782 | 28S | BK000964.3 | 2614 | 10736 | CCGG |
| U13369.1 | 3138 | 11072 | 28S | BK000964.3 | 2902 | 11024 | GCGG |
| U13369.1 | 3649 | 11583 | 28S | BK000964.3 | 3326 | 11448 | GCGG |
| U13369.1 | 670 | 8604 | 28S | BK000964.3 | 686 | 8808 | CCGG |
| U13369.1 | 2913 | 10847 | 28S | BK000964.3 | 2679 | 10801 | GCGG |
| U13369.1 | 3210 | 11144 | 28S | BK000964.3 | 2975 | 11097 | GCGG |
| U13369.1 | 2576 | 10510 | 28S | BK000964.3 | 2341 | 10463 | CCGA |
| U13369.1 | 1653 | 9587 | 28S | BK000964.3 | 1479 | 9601 | CCGA |
| U13369.1 | 2757 | 10691 | 28S | BK000964.3 | 2523 | 10645 | TCGC |
| U13369.1 | 3718 | 11652 | 28S | BK000964.3 | 3395 | 11517 | GCGC |
| U13369.1 | 1902 | 9836 | 28S | BK000964.3 | 1717 | 9839 | CCGA |
| U13369.1 | 653 | 8587 | 28S | BK000964.3 | 669 | 8791 | CCGG |
| U13369.1 | 1465 | 9399 | 28S | BK000964.3 | 1291 | 9413 | CCGC |
| U13369.1 | 1727 | 9661 | 28S | BK000964.3 | 1542 | 9664 | GCGA |
| U13369.1 | 1080 | 9014 | 28S | BK000964.3 | 948 | 9070 | GCGA |
| U13369.1 | 2908 | 10842 | 28S | BK000964.3 | 2674 | 10796 | GCGA |
| U13369.1 | 3961 | 11895 | 28S | BK000964.3 | 3638 | 11760 | GCGC |
| U13369.1 | 1974 | 9908 | 28S | BK000964.3 | 1789 | 9911 | TCGG |
| U13369.1 | 1175 | 9109 | 28S | BK000964.3 | 1020 | 9142 | GCGC |
| U13369.1 | 964 | 8898 | 28S | BK000964.3 | 876 | 8998 | GCGC |
| U13369.1 | 1461 | 9395 | 28S | BK000964.3 | 1287 | 9409 | TCGC |
| U13369.1 | 664 | 8598 | 28S | BK000964.3 | 680 | 8802 | CCGC |
| U13369.1 | 631 | 8565 | 28S | BK000964.3 | 648 | 8770 | TCGG |
| U13369.1 | 2492 | 10426 | 28S | BK000964.3 | 2260 | 10382 | CCGA |
| U13369.1 | 4233 | 12167 | 28S | BK000964.3 | 3915 | 12037 | CCGT |
| U13369.1 | 4123 | 12057 | 28S | BK000964.3 | 3805 | 11927 | GCGC |
| U13369.1 | 1796 | 9730 | 28S | BK000964.3 | 1611 | 9733 | CCGG |
| U13369.1 | 1908 | 9842 | 28S | BK000964.3 | 1723 | 9845 | CCGA |
| U13369.1 | 118 | 8052 | 28S | BK000964.3 | 118 | 8240 | CCGA |
| U13369.1 | 4867 | 12801 | 28S | BK000964.3 | 4547 | 12669 | GCGG |
| U13369.1 | 1180 | 9114 | 28S | BK000964.3 | 1025 | 9147 | CCGG |
| U13369.1 | 2380 | 10314 | 28S | BK000964.3 | 2148 | 10270 | CCGA |
| U13369.1 | 4909 | 12843 | 28S | BK000964.3 | 4591 | 12713 | ACGT |
| U13369.1 | 2496 | 10430 | 28S | BK000964.3 | 2264 | 10386 | TCGA |
| U13369.1 | 4135 | 12069 | 28S | BK000964.3 | 3817 | 11939 | CCGG |
| U13369.1 | 2445 | 10379 | 28S | BK000964.3 | 2213 | 10335 | GCGA |
| U13369.1 | 3789 | 11723 | 28S | BK000964.3 | 3466 | 11588 | GCGC |
| U13369.1 | 805 | 8739 | 28S | BK000964.3 | 776 | 8898 | GCGT |
| U13369.1 | 4848 | 12782 | 28S | BK000964.3 | 4528 | 12650 | TCGT |
| U13369.1 | 89 | 8023 | 28S | BK000964.3 | 89 | 8211 | ACGG |
| U13369.1 | 1683 | 9617 | 28S | BK000964.3 | 1509 | 9631 | TCGC |
| U13369.1 | 3269 | 11203 | 28S | BK000964.3 | 3034 | 11156 | GCGT |
| U13369.1 | 667 | 8601 | 28S | BK000964.3 | 683 | 8805 | CCGC |
| U13369.1 | 3293 | 11227 | 28S | BK000964.3 | 3058 | 11180 | CCGG |
| U13369.1 | 152 | 8086 | 28S | BK000964.3 | 155 | 8277 | GCGT |
| U13369.1 | 3124 | 11058 | 28S | BK000964.3 | 2885 | 11007 | GCGG |
| U13369.1 | 1590 | 9524 | 28S | BK000964.3 | 1416 | 9538 | TCGG |
| U13369.1 | 1468 | 9402 | 28S | BK000964.3 | 1294 | 9416 | CCGC |
| U13369.1 | 2118 | 10052 | 28S | BK000964.3 | 1925 | 10047 | GCGC |
| U13369.1 | 2703 | 10637 | 28S | BK000964.3 | 2469 | 10591 | GCGT |
| U13369.1 | 1677 | 9611 | 28S | BK000964.3 | 1503 | 9625 | GCGC |
| U13369.1 | 468 | 8402 | 28S | BK000964.3 | 476 | 8598 | GCGG |
| U13369.1 | 4786 | 12720 | 28S | BK000964.3 | 4465 | 12587 | CCGC |
| U13369.1 | 1538 | 9472 | 28S | BK000964.3 | 1364 | 9486 | GCGA |
| U13369.1 | 1866 | 9800 | 28S | BK000964.3 | 1681 | 9803 | GCGC |
| U13369.1 | 178 | 8112 | 28S | BK000964.3 | 181 | 8303 | CCGC |
| U13369.1 | 1470 | 9404 | 28S | BK000964.3 | 1296 | 9418 | GCGC |
| U13369.1 | 1719 | 9653 | 28S | BK000964.3 | 1534 | 9656 | CCGG |
| U13369.1 | 3153 | 11087 | 28S | BK000964.3 | 2916 | 11038 | GCGG |
| U13369.1 | 729 | 8663 | 28S | BK000964.3 | 745 | 8867 | CCGG |
| U13369.1 | 3866 | 11800 | 28S | BK000964.3 | 3543 | 11665 | GCGG |
| U13369.1 | 1279 | 9213 | 28S | BK000964.3 | 1106 | 9228 | GCGA |
| U13369.1 | 613 | 8547 | 28S | BK000964.3 | 630 | 8752 | GCGG |
| U13369.1 | 1226 | 9160 | 28S | BK000964.3 | 1068 | 9190 | ACGC |
| U13369.1 | 3228 | 11162 | 28S | BK000964.3 | 2993 | 11115 | GCGA |
| U13369.1 | 331 | 8265 | 28S | BK000964.3 | 339 | 8461 | CCGG |
| U13369.1 | 925 | 8859 | 28S | BK000964.3 | 835 | 8957 | TCGC |
| U13369.1 | 3958 | 11892 | 28S | BK000964.3 | 3635 | 11757 | CCGG |
| U13369.1 | 1473 | 9407 | 28S | BK000964.3 | 1299 | 9421 | CCGG |
| U13369.1 | 4754 | 12688 | 28S | BK000964.3 | 4438 | 12560 | CCGC |
| U13369.1 | 3000 | 10934 | 28S | BK000964.3 | 2765 | 10887 | CCGC |
| U13369.1 | 700 | 8634 | 28S | BK000964.3 | 716 | 8838 | GCGA |
| U13369.1 | 4969 | 12903 | 28S | BK000964.3 | 4651 | 12773 | TCGT |
| U13369.1 | 207 | 8141 | 28S | BK000964.3 | 210 | 8332 | TCGA |
| U13369.1 | 609 | 8543 | 28S | BK000964.3 | 626 | 8748 | GCGG |
| U13369.1 | 2452 | 10386 | 28S | BK000964.3 | 2220 | 10342 | CCGT |
| U13369.1 | 4831 | 12765 | 28S | BK000964.3 | 4511 | 12633 | CCGG |
| U13369.1 | 2277 | 10211 | 28S | BK000964.3 | 2045 | 10167 | CCGC |
| U13369.1 | 4791 | 12725 | 28S | BK000964.3 | 4470 | 12592 | GCGG |
| U13369.1 | 2932 | 10866 | 28S | BK000964.3 | 2698 | 10820 | GCGG |
| U13369.1 | 3527 | 11461 | 28S | BK000964.3 | 3200 | 11322 | CCGC |
| U13369.1 | 18 | 7952 | 28S | BK000964.3 | 18 | 8140 | ACGT |
| U13369.1 | 1285 | 9219 | 28S | BK000964.3 | 1112 | 9234 | TCGG |
| U13369.1 | 2010 | 9944 | 28S | BK000964.3 | 1825 | 9947 | CCGA |
| U13369.1 | 3386 | 11320 | 28S | BK000964.3 | 3143 | 11265 | GCGG |
| U13369.1 | 115 | 8049 | 28S | BK000964.3 | 115 | 8237 | GCGC |
| U13369.1 | 515 | 8449 | 28S | BK000964.3 | 527 | 8649 | CCGT |
| U13369.1 | 1598 | 9532 | 28S | BK000964.3 | 1424 | 9546 | CCGA |
| U13369.1 | 2718 | 10652 | 28S | BK000964.3 | 2484 | 10606 | TCGC |
| U13369.1 | 3728 | 11662 | 28S | BK000964.3 | 3405 | 11527 | ACGG |
| U13369.1 | 3188 | 11122 | 28S | BK000964.3 | 2953 | 11075 | CCGG |
| U13369.1 | 1339 | 9273 | 28S | BK000964.3 | 1166 | 9288 | GCGA |
| U13369.1 | 4860 | 12794 | 28S | BK000964.3 | 4540 | 12662 | ACGG |
| U13369.1 | 1194 | 9128 | 28S | BK000964.3 | 1040 | 9162 | CCGT |
| U13369.1 | 761 | 6288 | ITS1 | BK000964.3 | 662 | 6539 | CCGC |
| U13369.1 | 614 | 6141 | ITS1 | BK000964.3 | 535 | 6412 | GCGG |
| U13369.1 | 609 | 6136 | ITS1 | BK000964.3 | 530 | 6407 | CCGG |
| U13369.1 | 650 | 6177 | ITS1 | BK000964.3 | 569 | 6446 | CCGC |
| U13369.1 | 462 | 5989 | ITS1 | BK000964.3 | 380 | 6257 | CCGC |
| U13369.1 | 842 | 6369 | ITS1 | BK000964.3 | 738 | 6615 | GCGC |
| U13369.1 | 642 | 6169 | ITS1 | BK000964.3 | 561 | 6438 | TCGC |
| U13369.1 | 612 | 6139 | ITS1 | BK000964.3 | 533 | 6410 | GCGC |
| U13369.1 | 137 | 5664 | ITS1 | BK000964.3 | 59 | 5936 | TCGC |
| U13369.1 | 775 | 6302 | ITS1 | BK000964.3 | 676 | 6553 | GCGG |
| U13369.1 | 604 | 6131 | ITS1 | BK000964.3 | 525 | 6402 | GCGT |
| U13369.1 | 325 | 5852 | ITS1 | BK000964.3 | 246 | 6123 | GCGG |
| U13369.1 | 400 | 5927 | ITS1 | BK000964.3 | 320 | 6197 | CCGT |
| U13369.1 | 840 | 6367 | ITS1 | BK000964.3 | 736 | 6613 | CCGC |
| U13369.1 | 407 | 5934 | ITS1 | BK000964.3 | 325 | 6202 | CCGG |
| U13369.1 | 94 | 5621 | ITS1 | BK000964.3 | 20 | 5897 | GCGG |
| U13369.1 | 105 | 5632 | ITS1 | BK000964.3 | 33 | 5910 | CCGC |
| U13369.1 | 499 | 6026 | ITS1 | BK000964.3 | 418 | 6295 | TCGC |
| U13369.1 | 981 | 6508 | ITS1 | BK000964.3 | 876 | 6753 | CCGG |
| U13369.1 | 134 | 6913 | ITS2 | BK000964.3 | 132 | 7166 | GCGC |
| U13369.1 | 792 | 7571 | ITS2 | BK000964.3 | 785 | 7819 | GCGG |
| U13369.1 | 787 | 7566 | ITS2 | BK000964.3 | 780 | 7814 | ACGC |
| U13369.1 | 44 | 6823 | ITS2 | BK000964.3 | 36 | 7070 | GCGC |
| U13369.1 | 290 | 7069 | ITS2 | BK000964.3 | 290 | 7324 | CCGT |
| U13369.1 | 790 | 7569 | ITS2 | BK000964.3 | 783 | 7817 | CCGC |
| U13369.1 | 668 | 7447 | ITS2 | BK000964.3 | 670 | 7704 | GCGC |
| U13369.1 | 11 | 6790 | ITS2 | BK000964.3 | 10 | 7044 | TCGC |
| U13369.1 | 822 | 7601 | ITS2 | BK000964.3 | 815 | 7849 | CCGG |
| U13369.1 | 46 | 6825 | ITS2 | BK000964.3 | 38 | 7072 | GCGG |
| U13369.1 | 782 | 7561 | ITS2 | BK000964.3 | 775 | 7809 | GCGG |
| U13369.1 | 680 | 7459 | ITS2 | BK000964.3 | 683 | 7717 | CCGC |
| U13369.1 | 1062 | 7841 | ITS2 | BK000964.3 | 1044 | 8078 | TCGC |
| U13369.1 | 62 | 6841 | ITS2 | BK000964.3 | 54 | 7088 | TCGC |
| U13369.1 | 871 | 7650 | ITS2 | BK000964.3 | 861 | 7895 | GCGT |
| U13369.1 | 536 | 7315 | ITS2 | BK000964.3 | 537 | 7571 | CCGC |
| U13369.1 | 638 | 7417 | ITS2 | BK000964.3 | 640 | 7674 | CCGT |
| U13369.1 | 898 | 7677 | ITS2 | BK000964.3 | 889 | 7923 | GCGT |
| U13369.1 | 529 | 7308 | ITS2 | BK000964.3 | 531 | 7565 | CCGG |
| U13369.1 | 277 | 7056 | ITS2 | BK000964.3 | 277 | 7311 | TCGG |
| U13369.1 | 769 | 7548 | ITS2 | BK000964.3 | 762 | 7796 | CCGG |
| U13369.1 | 85 | 6864 | ITS2 | BK000964.3 | 89 | 7123 | CCGT |
| U13369.1 | 447 | 7226 | ITS2 | BK000964.3 | 454 | 7488 | GCGG |
| U13369.1 | 780 | 7559 | ITS2 | BK000964.3 | 773 | 7807 | CCGC |

TABLE 4
Primers used to measure methylation sites on
Models 1 and 2 listed in Table 2.

| Primer | | Seq ID |
|---|---|---|
| Name | Sequence | NO: |
| 45S-P-F1 | GTTGATATGTTGTTTTTTGG | 1 |
| 45S-P-R1 | AAACCAAAGAATAAAATTATAC | 2 |
| 45S-P-F2 | GTATAATTTTATTTGTTGGTTT | 3 |
| 45S-P-R2 | ACAAACAAAACTATCTACC | 4 |
| 45S-P-F3 | GGTAGATAGTTTTGTTTGT | 5 |
| 45S-P-R3 | TCCACCCACCTCCTTCCTTCC | 6 |
| 45S-P-F4 | GGAAGGAAGGAGGTGGGTGGA | 7 |
| 45S-P-R4 | CTTCTCCCCCCCAACCCC | 8 |
| 45S-P-F5 | GGGGTTGGGGGGGAGAAG | 9 |
| 45S-P-R5 | AAACGCTTTCCCAAAACCAAAC | 10 |
| 45S-P-F6 | GTTTGGTTTTGGGAAAGCGTTT | 11 |
| 45S-P-R6 | CCACAAACACGAAAACGATCCC | 12 |
| 45S-P-F7 | GGGATCGTTTTCGTGTTTGTGG | 13 |
| 45S-P-R7 | CCACCGACCCGATCCCCAAAAC | 14 |
| 45S-P-F8 | GTTTTGGGGATCGGGTCGGTGG | 15 |
| 45S-P-R8 | AAAAAAACAAACTCTCCCC | 16 |
| 45S-P-F9 | GGGGAGAGTTTGTTTTTTT | 17 |
| 45S-P-R9 | CAAAAATAACACACACCACAC | 18 |
| 45S-P-F10 | GTGTGGTGTGTGTTATTTTTG | 19 |
| 45S-P-R10 | CCCAACCACCCACCCCCCAC | 20 |
| 45S-P-F11 | GTGGGGGGTGGGTGGTTGGG | 21 |
| 45S-P-R11 | AATAAAATAAAACACAACAAACCCC | 22 |
| rDNA-C-F1 | GATTAAGTTATGTATGTTTAAGT | 23 |
| rDNA-C-R1 | CTACCTTCCTTAAATATAATAACC | 24 |
| rDNA-C-F2 | GGTTATTATATTTAAGGAAGGTAG | 25 |
| rDNA-C-R2 | TCCTATTCCATTATTCCTAACTAC | 26 |
| rDNA-C-F3 | GTAGTTAGGAATAATGGAATAGGA | 27 |
| rDNA-C-R3 | TCCTATTCCATTATTCCTAACTAC | 28 |
| rDNA-C-F4 | GGGGGGAGTATGGTTGTAAAGTTG | 29 |
| rDNA-C-R4 | CCACTTATCCCTCTAAAAAATTAAA | 30 |
| rDNA-C-F5 | TTTAATTTTTTAGAGGGATAAGTGG | 31 |
| rDNA-C-R5 | TTCATAAAAAATAATTACAATCCCC | 32 |
| rDNA-C-F6 | GGGGATTGTAATTATTTTTTATGAA | 33 |
| rDNA-C-R6 | TTACTTCCTCTAAATAATCAAATTC | 34 |
| rDNA-C-F7 | GAATTTGATTATTTAGAGGAAGTAA | 35 |
| rDNA-C-R7 | CCCTTCTTTCTCTCTCTCTCTCTCT | 36 |
| rDNA-C-F8 | AGAGAGAGAGAGAGAGAAAGAAGGG | 37 |
| rDNA-C-R8 | CCCCCCAAAAAATCTTTAAACCTCC | 38 |
| rDNA-C-F9 | GGAGGTTTAAAGATTTTTTGGGGGG | 39 |
| rDNA-C-R9 | TATCCTACAATTCACATTAATTCTC | 40 |
| rDNA-C-F10 | GAGAATTAATGTGAATTGTAGGATA | 41 |
| rDNA-C-R10 | ACTCTCTCTTTCCCTCTCC | 42 |
| rDNA-C-F11 | GGAGAGGGAAAGAGAGAGT | 43 |
| rDNA-C-R11 | AACAAAACCTCCA | 44 |
| rDNA-C-F12 | TGTTTTTTTTTTGAGGTTTTGTT | 45 |
| rDNA-C-R12 | TCTCCAAACC | 46 |
| rDNA-C-F13 | GAAATTAATTAGGATTTTTTTAGTAA | 47 |
| rDNA-C-R13 | CTTTAAACTACATTCCCAAACAACC | 48 |
| rDNA-C-F14 | GGTTGTTTGGGAATGTAGTTTAAAG | 49 |
| rDNA-C-R14 | CCTTCCCCACCAAACCTTCCCAACC | 50 |
| rDNA-C-F15 | GGTTGGGAAGGTTTGGTGGGGAAGG | 51 |
| rDNA-C-R15 | GGGGGGTTGGGTTATTTTTTTTA | 52 |
| rDNA-C-F16 | GGGGGGTTGGGTTATTTTTTTTA | 53 |
| rDNA-C-R16 | TTAAACTCCTTAATCCATATTTCAA | 54 |
| rDNA-C-F17 | TTGAAATATGGATTAAGGAGTTTAA | 55 |
| rDNA-C-R17 | AAAAAACCAACTACTAAATAATTC | 56 |
| rDNA-C-F18 | GAATTATTTAGTAGTTGGTTTTTT | 57 |
| rDNA-C-R18 | CCATCCATTTTCAAAACTAATTAAT | 58 |
| rDNA-C-F19 | ATTAATTAGTTTTGAAAATGGATGG | 59 |
| rDNA-C-R19 | TTTAAATATTTACTACTACCACCAA | 60 |
| rDNA-C-F20 | TTGGTGGTAGTAGTAAATATTTAAA | 61 |
| rDNA-C-R20 | AAATTTACACCCTCTCCCCC | 62 |
| rDNA-C-F21 | GGGGGAGAGGGTGTAAATTT | 63 |
| rDNA-C-R21 | CCCCAACCCTTCTCCCCCC | 64 |
| rDNA-C-F22 | GGGGGGAGAAGGGTTGGGG | 65 |
| rDNA-C-R22 | ATACTTTATTTTAATTAAACAATC | 66 |
| rDNA-C-F23 | GATTGTTTAATTAAAATAAAGTAT | 67 |
| rDNA-C-R23 | AAAACCTCCCACTTATTCTACACCT | 68 |
| rDNA-C-F24 | AGGTGTAGAATAAGTGGGAGGTTTT | 69 |
| rDNA-C-R24 | AAAAATTTCTATCCTCCCTAAACTC | 70 |
| rDNA-C-F25 | GAGTTTAGGGAGGATAGAAATTTTT | 71 |
| rDNA-C-R25 | CCTATTAATAAATAAACAATCCAAC | 72 |
| rDNA-C-F26 | GTTGGATTGTTTATTTATTAATAGG | 73 |
| rDNA-C-R26 | TTTCCCAAAACAAAAAACACTCC | 74 |
| rDNA-C-F27 | GGAGTGTTTTTTGTTTTGGGAAA | 75 |
| rDNA-C-R27 | AAAACTAACTTTCAATAAATC | 76 |

What is claimed is:

1) A method for determining a methylation age of a biological sample, the method comprising:
a. measuring the methylation level of a set of methylation sites on ribosomal DNA (rDNA) of the biological sample; and
b. determining the age of the biological sample using a statistical prediction algorithm based on the methylation level.

2) The method of claim 1, the method further comprising, prior to measuring, at least one of the steps of:
a. collecting a biological sample from the subject; or
b. extracting genomic DNA for the collected biological sample.

3) The method of claim 1, the method further comprising the step of:
comparing the methylation age of the subject to a chronological age of the subject;
wherein the Δage is the methylation age of the subject minus the chronological age of the subject.

4) The method of claim 1, wherein the biological sample is a blood sample or a tissue sample.

5) (canceled)

6) The method of claim 1, wherein the subject does or does not exhibit a risk factor of accelerated aging.

7) The method of claim 1, wherein the subject exhibits at least one risk factor of accelerated aging.

8) The method of claim 6, wherein the risk factor of accelerated aging is selected from the group consisting of: use of tobacco products, use of alcohol, exposure to environmental toxins, sedentary lifestyle, obesity, cancer, down syndrome, lack of nutritional intake, poor dietary habit, having complex diseases such as diabetes, CHD, hypertension, hyperlipidemia, and genetic risk predisposition.

9) The method of claim 1, wherein the set of methylation sites are the methylation sites in Table 1, Table 2, Table 5, Table 6, Table 7, or Table 8.

10) The method of claim 1, wherein the set of methylation sites comprise at least 90%, at least 80%, at least 70%, at least 60%, at least 50% of the sites of Table 1, Table 2, Table 5, Table 6, Table 7, or Table 8.

11) The method of claim 1, wherein the set of methylation sites comprise each of the sites of Table 1, Table 2, Table 5, Table 6, Table 7, or Table 8.

12) The method of claim 1, wherein the statistical prediction algorithm comprises:
a. identifying at least two coefficients found in Table 1, Table 2, Table 5, Table 6, Table 7, or Table 8 in a biological sample;
b. multiplying each of the at least two coefficients with its corresponding CpG's methylation level to output a value for each of the at least two coefficients;
c. find a sum of values of (b) for each identified coefficient;
d. adding a recalibration intercept to the summed values of (c);
e. calculating the natural exponentiation of (d), wherein the exponentiation is the predicted methylation age of the subject.

13) The method of claim 1, wherein a Δage greater than zero is an indicator of accelerated aging of the individual.

14) The method of claim 1, further comprising administering a pro-health therapy to a subject with a Δage greater than zero.

15) The method of claim 14, wherein the pro-health therapy is a therapy that decreases the methylation age of the subject.

16)-20) (canceled)

21) A kit comprising a set of probes for detecting methylation sites found in Table 1, Table 2, Table 5, Table 6, Table 7, or Table 8.

22) The kit of claim 21, wherein the set of probes comprise at least 90%, at least 80%, at least 70%, at least 60%, at least 50% of the sites of Table 1, Table 2, Table 5, Table 6, Table 7, or Table 8.

23) A system for determining a methylation age related property of a subject, the system comprising:
an array;
an array reader configured to output methylation levels;
a display;
a memory containing machine readable medium comprising machine executable code having stored thereon instructions for performing a method;
a control system coupled to the memory comprising one or more processors, the control system configured to execute the machine executable code to cause the control system to:
receive, from the array reader, a methylation data set related to a methylation level of a blood sample of a subject;
determine, based on the methylation data set, a methylation age related property using a regression model trained using subjects with an ethnicity that is the same as the subject's ethnicity; and
output, to the display, the methylation age related property.

24) The system of claim 23, wherein the methylation level of a blood sample of the subject is the method level of leukocytes of the subject.

25) The method of claim 4, wherein the blood is whole blood, peripheral blood, or cord blood.

26) The method of claim 4, wherein the tissue sample is selected from the group consisting of: skin tissue, breast tissue, ovarian tissue, liver tissue, kidney tissue, lung tissue, pancreatic tissue, thyroid tissue, thymus tissue, spleen tissue, bone marrow, lymphoid tissue, epithelial tissue, endothelial tissue, ectoderm tissue, nervous tissue, connective tissue, and mesoderm tissue.

27)-28) (canceled)
